(19) United States
(12) Patent Application Publication
(10) Pub. No.: US 2022/0307088 A1
(43) Pub. Date: Sep. 29, 2022

(54) B CELL-ENRICHED TUMOR MICROENVIRONMENTS

(71) Applicant: BostonGene Corporation, Waltham, MA (US)

(72) Inventors: Olga Kudriashova, Moscow (RU); Daria Melikhova, Moscow (RU); Nikita Kotlov, Moscow (RU); Mariia Gusakova, Krasnodar (RU); Svetlana Podsvirova, Moscow (RU)

(73) Assignee: BostonGene Corporation, Waltham, MA (US)

(21) Appl. No.: 17/690,757

(22) Filed: Mar. 9, 2022

Related U.S. Application Data
(60) Provisional application No. 63/158,816, filed on Mar. 9, 2021.

(51) Int. Cl. C12Q 1/6886; G16B 40/20

(52) U.S. Cl. CPC C12Q 1/6886; G16B 40/20; C12Q 2600/158

(57) ABSTRACT

Techniques for identifying a gastric cancer (GC) tumor microenvironment (TME) type for a subject having, suspected of having, or at risk of having gastric cancer. The techniques include: obtaining RNA expression data for the subject; generating a GC TME signature for the subject using the RNA expression data, the GC TME signature comprising gene group scores for respective gene groups in the at least some of the plurality of gene groups, the generating comprising: determining the gene group scores using the RNA expression data; and identifying, using the GC TME signature and from among a plurality of GC TME types, a GC TME type for the subject.

### CROSS REFERENCE TO RELATED APPLICATIONS

This application claims the benefit under 35 U.S.C. § 119(e) of the filing date of U.S. Provisional Application No. 63/158,816, filed Mar. 9, 2021, entitled “B CELL-ENRICHED TUMOR MICROENVIRONMENTS,” the entire contents of which are incorporated by reference herein.

### BACKGROUND

Correctly characterizing the type or types of cancer a patient or subject has and, potentially, selecting one or more effective therapies for the patient can be crucial for the survival and overall wellbeing of that patient. Advances in characterizing cancers, predicting prognoses, identifying effective therapies, and otherwise aiding in personalized care of patients with cancer are needed.

### SUMMARY

Aspects of the present disclosure relate to methods, systems, and computer-readable storage medium that can be used for identifying a gastric cancer (GC) tumor microenvironment (TME) type for a subject. In some aspects, the disclosure provides a method for identifying a gastric cancer (GC) tumor microenvironment (TME) type for a subject, comprising: using at least one computer hardware processor to perform: (a) obtaining RNA expression data for the subject, the RNA expression data indicating RNA expression levels for at least some genes in each group of at least some of a plurality of gene groups listed in Table 1; (b) generating a GC TME signature for the subject using the RNA expression data, the GC TME signature comprising gene group scores for respective gene groups in the at least some of the plurality of gene groups, the generating comprising: determining the gene group scores using the RNA expression levels; and (c) identifying, using the GC TME signature and from among a plurality of GC TME types, a GC TME type for the subject.

Aspects of the present disclosure include a system, comprising: at least one computer hardware processor; and at least one computer-readable storage medium storing processor-executable instructions that, when executed by the at least one computer hardware processor, cause the at least one computer hardware processor to perform a method for identifying a gastric cancer (GC) tumor microenvironment (TME) type for a subject, the method comprising: obtaining RNA expression data for the subject, the RNA expression data indicating RNA expression levels for at least some genes in each group of at least some of a plurality of gene groups listed in Table 1; generating a GC TME signature for the subject using the RNA expression data, the GC TME signature comprising gene group scores for respective gene groups in the at least some of the plurality of gene groups, the generating comprising: determining the gene group scores using the RNA expression levels; and identifying, using the GC TME signature and from among a plurality of GC TME types, a GC TME type for the subject.

Aspects of the present disclosure include at least one computer-readable storage medium storing processor-executable instructions that, when executed by the at least one computer hardware processor, cause the at least one computer hardware processor to perform a method for identifying a gastric cancer (GC) tumor microenvironment (TME) type for a subject, the method comprising: obtaining RNA expression data for the subject, the RNA expression data indicating RNA expression levels for at least some genes in each group of at least some of a plurality of gene groups listed in Table 1; generating a GC TME signature for the subject using the RNA expression data, the GC TME signature comprising gene group scores for respective gene groups in the at least some of the plurality of gene groups, the generating comprising: determining the gene group scores using the RNA expression levels; and identifying, using the GC TME signature and from among a plurality of GC TME types, a GC TME type for the subject.

In some embodiments, the subject has, is suspected of having, or is at risk of having gastric cancer.

In some embodiments, the method described herein further comprises identifying the subject as having GC TME type E; and when the subject is identified as having the GC TME type E, administering an immunotherapy to the subject.

In some embodiments, obtaining the RNA expression data for the subject comprises obtaining sequencing RNA data previously obtained by sequencing a biological sample obtained from the subject.

In some embodiments, the sequencing data comprises at least 1 million reads, at least 5 million reads, at least 10 million reads, at least 20 million reads, at least 50 million reads, or at least 100 million reads.

In some embodiments, the sequencing data comprises whole exome sequencing (WES) data, bulk RNA sequencing (RNA-seq) data, single cell RNA sequencing (scRNA-seq) data, or next generation sequencing (NGS) data. In some embodiments, the sequencing data comprises microarray data.

In some embodiments, the method described herein further comprises normalizing the RNA expression data to transcripts per million (TPM) units prior to generating the GC TME signature.

In some embodiments, obtaining the RNA expression data for the subject comprises sequencing a biological sample obtained from the subject. In some embodiments, the biological sample comprises gastrointestinal tissue of the subject. In some embodiments, the biological sample comprises tumor tissue of the subject.

In some embodiments, the RNA expression levels comprise RNA expression levels for at least three genes from each of at least two of the following gene groups: (i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (ii) T cells group: TBX21, ITK, CD3D, CD3E, CD3G, TRAC, TRBC1, TRBC2, CD28, CD5, TRAT1; (iii) B cells group: CD19, MS4A1, TNFRSF13C, CR2, TNFRSF17, TNFRSF13B, CD22, CD79A, CD79B, BLK, FCRL5, PAX5, STAP1; (iv) Treg group: FOXP3, CTLA4, IL10, TNFRSF18, CCR8, IKZF4, IKZF2; (v) MDSC group: IDO1, ARG1, IL10, CYBB, PTGS2, IL4I1, IL6; (vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (vii) Proliferation rate group: MK167, ESCO2, CETN3, CDK2, CCND1, CCNE1, AURKA, AURKB, E2F1, MYBL2, BUB1, CCNB1, MCM2, MCM6; and (viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, the RNA expression levels comprise RNA expression levels for each of the genes from each of the following gene groups: (i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (ii) T cells group: TBX21, ITK, CD3D, CD3E, CD3G, TRAC, TRBC1, TRBC2, CD28, CD5, TRAT1; (iii) B cells group: CD19, MS4A1, TNFRSF13C, CR2, TNFRSF17, TNFRSF13B, CD22, CD79A, CD79B, BLK, FCRL5, PAX5, STAP1; (iv) Treg group: FOXP3, CTLA4, IL10, TNFRSF18, CCR8, IKZF4, IKZF2; (v) MDSC group: IDO1, ARG1, IL10, CYBB, PTGS2, IL4I1, IL6; (vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (vii) Proliferation rate group: MK167, ESCO2, CETN3, CDK2, CCND1, CCNE1, AURKA, AURKB, E2F1, MYBL2, BUB1, CCNB1, MCM2, MCM6; and (viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, the RNA expression levels comprise RNA expression levels for at least three genes from each of at least two of the following gene groups: (a) MHC I group: HLA-A, HLA-B, HLA-C, B2M, TAP1, TAP2, NLRC5, TAPBP; (b) MHC II group: HLA-DRA, HLA-DRB1, HLA-DMA, HLA-DPA1, HLA-DPB1, HLA-DMB, HLA-DQB1, HLA-DQA1, CIITA; (c) Coactivation molecules group: CD28, CD40, TNFRSF4, ICOS, TNFRSF9, CD27, CD80, CD86, CD40LG, CD83, TNFSF4, ICOSLG, TNFSF9, CD70; (d) Effector cells group: IFNG, GZMA, GZMB, PRF1, GZMK, ZAP70, GNLY, FASLG, TBX21, EOMES, CD8A, CD8B; (e) T cell traffic group: CXCL9, CXCL10, CXCL11, CX3CL1, CCL3, CCL4, CX3CR1, CXCL16, CXCR6; (f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (g) T cells group: TBX21, ITK, CD3D, CD3E, CD3G, TRAC, TRBC1, TRBC2, CD28, CD5, TRAT1; (h) B cells group: CD19, MS4A1, TNFRSF13C, CR2, TNFRSF17, TNFRSF13B, CD22, CD79A, CD79B, BLK, FCRL5, PAX5, STAP1; (i) M1 signature group: NOS2, TNF, IL1B, SOCS3, CMKLR1, IRF5, IL12A, IL12B, IL23A; (j) Antitumor cytokines group: TNF, IFNB1, IFNA2, CCL3, TNFSF10, IL21; (k) Checkpoint inhibition group: PDCD1, CD274, CTLA4, LAG3, PDCD1LG2, BTLA, HAVCR2, TIGIT, VSIR; (l) Treg group: FOXP3, CTLA4, IL10, TNFRSF18, CCR8, IKZF4, IKZF2; (m) Neutrophil signature group: MPO, ELANE, PRTN3, CTSG, CXCR1, CXCR2, FCGR3B, CD177, FFAR2, PGLYRP1; (n) MDSC group: IDO1, ARG1, IL10, CYBB, PTGS2, IL4I1, IL6; (o) M2 signature group: IL10, MRC1, MSR1, CD163, CSF1R, IL4I1, SIGLEC1, CD68; (p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (q) Angiogenesis group: VEGFA, VEGFB, VEGFC, PDGFC, CXCL8, CXCR2, FLT1, PGF, KDR, ANGPT1, ANGPT2, TEK, VWF, CDH5; (r) Proliferation rate group: MK167, ESCO2, CETN3, CDK2, CCND1, CCNE1, AURKA, AURKB, E2F1, MYBL2, BUB1, CCNB1, MCM2, MCM6; and (s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining the gene group scores comprises: determining a respective gene group score for each of at least two of the following gene groups, using, for a particular gene group, RNA expression levels for at least three genes in the particular gene group to determine the gene group score for the particular group, the gene groups including: (i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (ii) T cells group: TBX21, ITK, CD3D, CD3E, CD3G, TRAC, TRBC1, TRBC2, CD28, CD5, TRAT1; (iii) B cells group: CD19, MS4A1, TNFRSF13C, CR2, TNFRSF17, TNFRSF13B, CD22, CD79A, CD79B, BLK, FCRL5, PAX5, STAP1; (iv) Treg group: FOXP3, CTLA4, IL10, TNFRSF18, CCR8, IKZF4, IKZF2; (v) MDSC group: IDO1, ARG1, IL10, CYBB, PTGS2, IL4I1, IL6; (vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (vii) Proliferation rate group: MK167, ESCO2, CETN3, CDK2, CCND1, CCNE1, AURKA, AURKB, E2F1, MYBL2, BUB1, CCNB1, MCM2, MCM6; and (viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining the gene group scores comprises: determining a respective gene group score for each of the following gene groups, using, for each gene group, RNA expression levels for each of the genes in each gene group to determine the gene group score for each particular group, the gene groups including: (i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (ii) T cells group: TBX21, ITK, CD3D, CD3E, CD3G, TRAC, TRBC1, TRBC2, CD28, CD5, TRAT1; (iii) B cells group: CD19, MS4A1, TNFRSF13C, CR2, TNFRSF17, TNFRSF13B, CD22, CD79A, CD79B, BLK, FCRL5, PAX5, STAP1; (iv) Treg group: FOXP3, CTLA4, IL10, TNFRSF18, CCR8, IKZF4, IKZF2; (v) MDSC group: IDO1, ARG1, IL10, CYBB, PTGS2, IL4I1, IL6; (vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (vii) Proliferation rate group: MK167, ESCO2, CETN3, CDK2, CCND1, CCNE1, AURKA, AURKB, E2F1, MYBL2, BUB1, CCNB1, MCM2, MCM6; and (viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining the gene group scores comprises determining a first score of a first gene group using a single-sample GSEA (ssGSEA) technique from RNA expression levels for at least some of the genes in one of the following gene groups: (i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (ii) T cells group: TBX21, ITK, CD3D, CD3E, CD3G, TRAC, TRBC1, TRBC2, CD28, CD5, TRAT1; (iii) B cells group: CD19, MS4A1, TNFRSF13C, CR2, TNFRSF17, TNFRSF13B, CD22, CD79A, CD79B, BLK, FCRL5, PAX5, STAP1; (iv) Treg group: FOXP3, CTLA4, IL10, TNFRSF18, CCR8, IKZF4, IKZF2; (v) MDSC group: IDO1, ARG1, IL10, CYBB, PTGS2, IL4I1, IL6; (vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (vii) Proliferation rate group: MK167, ESCO2, CETN3, CDK2, CCND1, CCNE1, AURKA, AURKB, E2F1, MYBL2, BUB1, CCNB1, MCM2, MCM6; and (viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining the gene group scores comprises determining the gene group scores, using a single-sample GSEA (ssGSEA) technique, from RNA expression levels for each of the genes in each of the following gene groups: (i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (ii) T cells group: TBX21, ITK, CD3D, CD3E, CD3G, TRAC, TRBC1, TRBC2, CD28, CD5, TRAT1; (iii) B cells group: CD19, MS4A1, TNFRSF13C, CR2, TNFRSF17, TNFRSF13B, CD22, CD79A, CD79B, BLK, FCRL5, PAX5, STAP1; (iv) Treg group: FOXP3, CTLA4, IL10, TNFRSF18, CCR8, IKZF4, IKZF2; (v) MDSC group: IDO1, ARG1, IL10, CYBB, PTGS2, IL4I1, IL6; (vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (vii) Proliferation rate group: MK167, ESCO2, CETN3, CDK2, CCND1, CCNE1, AURKA, AURKB, E2F1, MYBL2, BUB1, CCNB1, MCM2, MCM6; and (viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining the gene group scores comprises: determining a respective gene group score for each of at least two of the following gene groups, using, for a particular gene group, RNA expression levels for at least three genes in the particular gene group to determine the gene group score for the particular group, the gene groups including: (a) MHC I group: HLA-A, HLA-B, HLA-C, B2M, TAP1, TAP2, NLRC5, TAPBP; (b) MHC II group: HLA-DRA, HLA-DRB1, HLA-DMA, HLA-DPA1, HLA-DPB1, HLA-DMB, HLA-DQB1, HLA-DQA1, CIITA; (c) Coactivation molecules group: CD28, CD40, TNFRSF4, ICOS, TNFRSF9, CD27, CD80, CD86, CD40LG, CD83, TNFSF4, ICOSLG, TNFSF9, CD70; (d) Effector cells group: IFNG, GZMA, GZMB, PRF1, GZMK, ZAP70, GNLY, FASLG, TBX21, EOMES, CD8A, CD8B; (e) T cell traffic group: CXCL9, CXCL10, CXCL11, CX3CL1, CCL3, CCL4, CX3CR1, CXCL16, CXCR6; (f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (g) T cells group: TBX21, ITK, CD3D, CD3E, CD3G, TRAC, TRBC1, TRBC2, CD28, CD5, TRAT1; (h) B cells group: CD19, MS4A1, TNFRSF13C, CR2, TNFRSF17, TNFRSF13B, CD22, CD79A, CD79B, BLK, FCRL5, PAX5, STAP1; (i) M1 signature group: NOS2, TNF, IL1B, SOCS3, CMKLR1, IRF5, IL12A, IL12B, IL23A; (j) Antitumor cytokines group: TNF, IFNB1, IFNA2, CCL3, TNFSF10, IL21; (k) Checkpoint inhibition group: PDCD1, CD274, CTLA4, LAG3, PDCD1LG2, BTLA, HAVCR2, TIGIT, VSIR; (l) Treg group: FOXP3, CTLA4, IL10, TNFRSF18, CCR8, IKZF4, IKZF2; (m) Neutrophil signature group: MPO, ELANE, PRTN3, CTSG, CXCR1, CXCR2, FCGR3B, CD177, FFAR2, PGLYRP1; (n) MDSC group: IDO1, ARG1, IL10, CYBB, PTGS2, IL4I1, IL6; (o) M2 signature group: IL10, MRC1, MSR1, CD163, CSF1R, IL4I1, SIGLEC1, CD68; (p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (q) Angiogenesis group: VEGFA, VEGFB, VEGFC, PDGFC, CXCL8, CXCR2, FLT1, PGF, KDR, ANGPT1, ANGPT2, TEK, VWF, CDH5; (r) Proliferation rate group: MK167, ESCO2, CETN3, CDK2, CCND1, CCNE1, AURKA, AURKB, E2F1, MYBL2, BUB1, CCNB1, MCM2, MCM6; and (s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining the gene group scores comprises determining the gene group scores, using a single-sample GSEA (ssGSEA) technique, from RNA expression levels for at least some of the genes in each one of the following gene groups: (a) MHC I group: HLA-A, HLA-B, HLA-C, B2M, TAP1, TAP2, NLRC5, TAPBP; (b) MHC II group: HLA-DRA, HLA-DRB1, HLA-DMA, HLA-DPA1, HLA-DPB1, HLA-DMB, HLA-DQB1, HLA-DQA1, CIITA; (c) Coactivation molecules group: CD28, CD40, TNFRSF4, ICOS, TNFRSF9, CD27, CD80, CD86, CD40LG, CD83, TNFSF4, ICOSLG, TNFSF9, CD70; (d) Effector cells group: IFNG, GZMA, GZMB, PRF1, GZMK, ZAP70, GNLY, FASLG, TBX21, EOMES, CD8A, CD8B; (e) T cell traffic group: CXCL9, CXCL10, CXCL11, CX3CL1, CCL3, CCL4, CX3CR1, CXCL16, CXCR6; (f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (g) T cells group: TBX21, ITK, CD3D, CD3E, CD3G, TRAC, TRBC1, TRBC2, CD28, CD5, TRAT1; (h) B cells group: CD19, MS4A1, TNFRSF13C, CR2, TNFRSF17, TNFRSF13B, CD22, CD79A, CD79B, BLK, FCRL5, PAX5, STAP1; (i) M1 signature group: NOS2, TNF, IL1B, SOCS3, CMKLR1, IRF5, IL12A, IL12B, IL23A; (j) Antitumor cytokines group: TNF, IFNB1, IFNA2, CCL3, TNFSF10, IL21; (k) Checkpoint inhibition group: PDCD1, CD274, CTLA4, LAG3, PDCD1LG2, BTLA, HAVCR2, TIGIT, VSIR; (l) Treg group: FOXP3, CTLA4, IL10, TNFRSF18, CCR8, IKZF4, IKZF2; (m) Neutrophil signature group: MPO, ELANE, PRTN3, CTSG, CXCR1, CXCR2, FCGR3B, CD177, FFAR2, PGLYRP1; (n) MDSC group: IDO1, ARG1, IL10, CYBB, PTGS2, IL4I1, IL6; (o) M2 signature group: IL10, MRC1, MSR1, CD163, CSF1R, IL4I1, SIGLEC1, CD68; (p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (q) Angiogenesis group: VEGFA, VEGFB, VEGFC, PDGFC, CXCL8, CXCR2, FLT1, PGF, KDR, ANGPT1, ANGPT2, TEK, VWF, CDH5; (r) Proliferation rate group: MK167, ESCO2, CETN3, CDK2, CCND1, CCNE1, AURKA, AURKB, E2F1, MYBL2, BUB1, CCNB1, MCM2, MCM6; and (s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining the gene group scores comprises determining the gene group scores, using a single-sample GSEA (ssGSEA) technique, from RNA expression levels for each of the genes in each of the following gene groups: (a) MHC I group: HLA-A, HLA-B, HLA-C, B2M, TAP1, TAP2, NLRC5, TAPBP; (b) MHC II group: HLA-DRA, HLA-DRB1, HLA-DMA, HLA-DPA1, HLA-DPB1, HLA-DMB, HLA-DQB1, HLA-DQA1, CIITA; (c) Coactivation molecules group: CD28, CD40, TNFRSF4, ICOS, TNFRSF9, CD27, CD80, CD86, CD40LG, CD83, TNFSF4, ICOSLG, TNFSF9, CD70; (d) Effector cells group: IFNG, GZMA, GZMB, PRF1, GZMK, ZAP70, GNLY, FASLG, TBX21, EOMES, CD8A, CD8B; (e) T cell traffic group: CXCL9, CXCL10, CXCL11, CX3CL1, CCL3, CCL4, CX3CR1, CXCL16, CXCR6; (f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; (g) T cells group: TBX21, ITK, CD3D, CD3E, CD3G, TRAC, TRBC1, TRBC2, CD28, CD5, TRAT1; (h) B cells group: CD19, MS4A1, TNFRSF13C, CR2, TNFRSF17, TNFRSF13B, CD22, CD79A, CD79B, BLK, FCRL5, PAX5, STAP1; (i) M1 signature group: NOS2, TNF, IL1B, SOCS3, CMKLR1, IRF5, IL12A, IL12B, IL23A; (j) Antitumor cytokines group: TNF, IFNB1, IFNA2, CCL3, TNFSF10, IL21; (k) Checkpoint inhibition group: PDCD1, CD274, CTLA4, LAG3, PDCD1LG2, BTLA, HAVCR2, TIGIT, VSIR; (l) Treg group: FOXP3, CTLA4, IL10, TNFRSF18, CCR8, IKZF4, IKZF2; (m) Neutrophil signature group: MPO, ELANE, PRTN3, CTSG, CXCR1, CXCR2, FCGR3B, CD177, FFAR2, PGLYRP1; (n) MDSC group: IDO1, ARG1, IL10, CYBB, PTGS2, IL4I1, IL6; (o) M2 signature group: IL10, MRC1, MSR1, CD163, CSF1R, IL4I1, SIGLEC1, CD68; (p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (q) Angiogenesis group: VEGFA, VEGFB, VEGFC, PDGFC, CXCL8, CXCR2, FLT1, PGF, KDR, ANGPT1, ANGPT2, TEK, VWF, CDH5; (r) Proliferation rate group: MK167, ESCO2, CETN3, CDK2, CCND1, CCNE1, AURKA, AURKB, E2F1, MYBL2, BUB1, CCNB1, MCM2, MCM6; and (s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, generating the GC TME signature further comprises normalizing the gene group scores. In some embodiments, the normalizing comprises applying rank estimation and/or median scaling to the gene group scores.

In some embodiments, the plurality of GC TME types is associated with a respective plurality of GC TME signature clusters, wherein identifying, using the GC TME signature and from among a plurality of GC TME types, the GC TME type for the subject comprises: associating the GC TME signature of the subject with a particular one of the plurality of GC TME signature clusters; and identifying the GC TME type for the subject as the GC TME type corresponding to the particular one of the plurality of GC TME signature clusters to which the GC TME signature of the subject is associated.

In some embodiments, the method described herein further comprises generating the plurality of GC TME signature clusters, comprising: obtaining multiple sets of RNA expression data by sequencing biological samples from multiple respective subjects, each of the multiple sets of RNA expression data indicating RNA expression levels for at least some genes in each of the at least some of the plurality of gene groups listed in Table 1; generating multiple GC TME signatures from the multiple sets of RNA expression data, each of the multiple GC TME signatures comprising gene group scores for respective gene groups in the plurality of gene groups, the generating comprising, for each particular one of the multiple GC TME signatures, determining the GC TME signature by determining the gene group scores using the RNA expression levels in the particular set of RNA expression data for which the particular one GC TME signature is being generated; and clustering the multiple GC signatures to obtain the plurality of GC TME signature clusters.

In some embodiments, the clustering comprises dense clustering, spectral clustering, k-means clustering, hierarchical clustering, and/or an agglomerative clustering. In some embodiments, the hierarchical clustering is performed using a Louvain community detection algorithm.

In some embodiments, the method described herein further comprises updating the plurality of GC TME signature clusters using the GC TME signature of the subject, wherein the GC TME signature of the subject is one of a threshold number GC TME signatures for a threshold number of subjects, wherein when the threshold number of GC TME signatures is generated the GC TME signature clusters are updated.

In some embodiments, the threshold number of GC TME signatures is at least 50, at least 75, at least 100, at least 200, at least 500, at least 1000, or at least 5000 GC TME signatures.

In some embodiments, the updating comprises applying a dense clustering, spectral clustering, k-means clustering, hierarchical clustering, and/or agglomerative clustering. In some embodiments, the hierarchical clustering is performed using a Louvain community detection algorithm.

In some embodiments, the method described herein further comprises determining an GC TME type of a second subject, wherein the GC TME type of the second subject is identified using the updated GC TME signature clusters, wherein the identifying comprises: determining an GC TME signature of the second subject from RNA expression data obtained by sequencing a biological sample obtained from the second subject; associating the GC TME signature of the second subject with a particular one of the plurality of the updated GC TME signature clusters; and identifying the GC TME type for the second subject as the GC TME type corresponding to the particular one of the plurality of updated GC TME signature clusters to which the GC TME signature of the second subject is associated.

In some embodiments, the plurality of a plurality of GC TME types comprises: GC TME type A, GC TME type B, GC TME type C, GC TME type D, and GC TME type E.

In some embodiments, the method described herein further comprises identifying the subject as having tertiary lymphoid structures (TLS) when the subject is identified as having GC TME type E.

In some embodiments, the method described herein further comprises identifying the subject as having an increased likelihood of having a good prognosis. In some embodiments, increased likelihood of having a good prognosis is as measured by overall survival (OS) or progression-free survival (PFS) when the subject is identified as having GC TME type E.

In some embodiments, the method described herein further comprises administering an immunotherapy to the subject. The immunotherapy may be administered when the subject is identified as having GC TME type E. The immunotherapy may be administered when the subject is identified as having TLS. In some embodiments, the immunotherapy comprises a PD1 inhibitor. In some embodiments, the PD1 inhibitor comprises pembrolizumab.

### BRIEF DESCRIPTION OF DRAWINGS

FIG. 1 is a diagram depicting a flowchart of an illustrative process for determining a gastric cancer (GC) tumor microenvironment (TME) type for a subject having, suspected of having, or at risk of having a GC, according to some embodiments of the technology as described herein.

FIG. 2 is a diagram depicting a flowchart of an illustrative process for processing sequencing data to obtain RNA expression data, according to some embodiments of the technology as described herein.

FIG. 3 is a diagram depicting an illustrative technique for determining gene group scores, according to some embodiments of the technology as described herein.

FIG. 4 is a diagram depicting an illustrative technique for identifying a GC TME type for a subject using a GC TME signature, according to some embodiments of the technology as described herein.

FIG. 5A provides an exemplary heatmap of GC samples classified into five distinct GC TME type (A, B, C, D, E) using example GC TME signatures comprising 20 gene group scores, according to some aspects of the technology as described herein. Gene groups are listed vertically on the left side of the heatmap. Each column represents one sample. Panel on the top corresponds to the sample annotation: MFP—Gastric cancer type; Cohort—ID of dataset; Lauren—histological subtype of stomach cancer; Stage—tumor stage.

FIG. 5B provides a heatmap of pairwise gene group score correlation for the gene groups listed in Table 1.

FIG. 6 provides an example of gene group score comparison across GC TME types, according to some embodiments of the technology described herein.

FIG. 7 provides an exemplary heatmap of GC TME type gene group scores and cell deconvolution across the samples, according to some aspects of the technology as described herein. The columns represent single samples and are ordered by the GC TME types as follows: B, E, D, A, and C. Panel on the top corresponds to the sample annotation: MFP-GC TME type; Lauren—histological subtype of stomach cancer; Cohort—ID of the dataset. The bottom bar plots represent the cell composition fraction. Each color corresponds to one cell type (according to the legend on the right). Boxplots on the right represent comparison of cell composition across GC TME types.

FIGS. 8A-8F provides representative data indicating The Cancer Genome Atlas (TCGA) histological data supports GC TME types identified by methods described herein, according to some embodiments of the technology described herein. FIG. 8A shows GC TME type A (Mesenchymal, EMT) and GC TME type C (Fibrotic) histology slides indicate a high level of fibrous tissue and fibroblasts, with lower purity (e.g., malignant cell content) (left); GC TME type B (Immune Enriched, Non-Fibrotic) histology slides indicate lymphoid infiltration and an inflamed immunophenotype (second from left); GC TME type D (Depleted) histology slides indicate high purity, a desert of immune cells, and low fibroblast numbers (second from right); GC TME type E (B cell Enriched) histology slides indicate lymphoid infiltration (inflamed immunophenotype) and the presence of germinative centers (e.g., high level of B cells, indicated by arrow) (right). FIG. 8B shows a representation of the relative cell type content of GC TME type A. FIG. 8C shows a representation of the relative cell type content of GC TME type B.

FIG. 8D shows a representation of the relative cell type content of GC TME type C. FIG. 8E shows a representation of the relative cell type content of GC TME type D. FIG. 8F shows a representation of the relative cell type content of GC TME type E.

FIG. 9 provides representative data indicating survival and progression analysis across different GC TME types, according to some embodiments of the technology described herein.

FIG. 10 provides representative data indicating that most microsatellite-unstable/instability (MSI) samples were classified as having a GC TME type B while most microsatellite stable (MSS) samples were classified as having a GC TME type A, in accordance with some embodiments, of the technology described herein.

FIGS. 11A and 11B provide representative data of mutation burden across GC TME types. FIG. 11A shows that GC TME type B (i.e., immune enriched type) has the highest mutation load (ML). FIG. 11B shows that the highest purity (e.g., cellularity or amount of malignant cells) (left) and the highest ploidy (right) were in the GC TME type D based on the ACGR cohort data.

FIG. 12 provides representative data indicating strong relationship between EBV positive status and GC TME B and E types (immune enriched and B cell enriched). 1.0=EBV positive; 00.0=EBV negative.

FIG. 13 depicts an illustrative implementation of a computer system that may be used in connection with some embodiments of the technology described herein.

### DETAILED DESCRIPTION

Aspects of the disclosure relate to methods for characterizing subjects having certain cancers, for example gastric cancers. The disclosure is based, in part, on methods for determining the tumor microenvironment (TME) type of a subject's gastric cancer. In some embodiments the methods comprise identifying a subject as having a particular gastric cancer (GC) TME type based upon a GC TME signature computed for the subject from their RNA expression data. The GC TME signature may comprise gene group scores for gene groups that are associated with malignant (e.g., cancer cells, tumor cells, etc.) and certain immune cells (e.g., T cells, B cells, Treg cells, etc.). The GC TME type identified for the subject may have various prognostic, diagnostic, and/or therapeutic applications. For example, in some embodiments, methods developed by the inventors and described herein are useful for identifying a subject's prognosis based upon the GC TME type identified for the subject.

Gastric cancer is the fifth most common cancer in the world. The 5-year overall survival rate for patients having advanced gastric cancer is approximately ˜20%. Gastric cancer tumors are highly heterogeneous, and therefore present significant therapeutic challenges.

Methods of classifying gastric cancer generally include histological analysis and/or molecular analysis. Histological analysis comprises obtaining a gastric tumor biopsy sample and analyzing the tissue to ascertain the presence of certain cell morphologies, and characterizing the tumor based upon the analysis. One example of histological analysis of gastric cancers is Lauren classification, for example as described by Ma et al. Oncol Lett. 2016 May; 11(5):2959-2964. Histological analysis is generally viewed as expensive, laborious, and time-consuming. Additionally, histological classification is typically reliant on a subjective determination made by laboratory personnel, and therefore the possibility of inaccuracy or human-error exists.

Efforts have also been made to classify gastric cancers using molecular techniques in order to address the limitations of histological analysis. However, many studies performing molecular analysis on gastric cancer tissue are limited to gene expression studies of the tumor microenvironment, in particular tumor infiltrating immune lymphocytes (TIILs), for example as described by Hennequin et al. 2016 OncoImmunology, 5:2, DOI: 10.1080/2162402X.2015.1054598. Accordingly, the inventors have recognized that there is a need to develop methods for molecular characterization of GC types specifically based upon the underlying biology of both the tumor microenvironment and malignant cells, rather than more broadly defined cancer biomarkers.

Aspects of the disclosure relate to statistical techniques for analyzing expression data (e.g., RNA expression data), which was obtained from a biological sample obtained from a subject that has gastric cancer (GC), is suspected of having GC, or is at risk of developing GC, in order to generate a GC tumor microenvironment (TME) signature for the subject (termed an “GC TME signature” herein) and use this signature to identify a particular GC TME type that the subject may have.

The inventors have recognized that a combination of certain gene group scores determined using RNA expression data of a subject (e.g., gene group scores for at least some of the gene groups listed in Table 1) may be combined to form a GC TME signature that characterizes patients having GC more accurately than previously developed methods. A GC TME signature comprising a combination of gene group scores from gene groups associated with the tumor microenvironment, and gene group scores from gene groups associated with malignant cells, in turn, may be used to identify the subject as having a particular gastric cancer (GC) tumor microenvironment (TME) type.

The use of GC TME signatures comprising the combinations of gene group scores described by the disclosure represents an improvement over previously described GC molecular biomarkers or tumor microenvironment analyses because the specific groups of genes used to produce the GC TME signatures described herein better reflect the molecular tumor microenvironments of GC because these gene groups are associated with 1) gastric cancer tumor cells, and 2) gastric cancer tumor microenvironment. These focused combinations of gene groups (e.g., gene groups consisting of some or all of the genes listed in Table 1) are unconventional, and differ from previously described molecular signatures, which attempt to incorporate expression data from either very large numbers of genes, or only account for TIILs.

The GC TME typing methods described herein have several utilities. For example, identifying a subject's GC TME type using methods described herein may allow for the subject to be diagnosed as having (or being at a high risk of developing) an aggressive form of GC at a timepoint that is not possible with previously described GC characterization methods. Earlier detection of aggressive GC types, enabled by the GC TME signatures described herein, improve the patient diagnostic technology by enabling earlier chemotherapeutic intervention for patients than currently possible for patients tested for GC using other methods (e.g., histological analysis).

As described herein, the inventors have also determined that subjects identified by methods described herein as having GC TME type E are characterized as being more likely to have tertiary lymphoid structures (TLS), and thus having a good prognosis. Subjects having GC TME type E, and therefore, more likely to have TLS, may be treated with cancer immunotherapy. Identifying a subject as having TLS without the need to perform a biopsy is advantageous because it allows the patient to avoid surgical procedures while still providing medical providers the benefit of identifying the presence of TLS in the subject (e.g., a subject having TLS may be treated using an immunotherapy). Conversely, the inventors have determined that identifying a subject as having GC TME type A or GC TME type C using methods described herein, are less likely to have TLS and/or are more likely to have an aggressive or treatment-resistant form of GC. Thus, the techniques developed by the inventors and described herein improve patient treatment and associated outcomes by increasing patient comfort, and avoiding toxic side effects of chemotherapy that is not expected to be effective for the subject.

Gastric Cancers Aspects of the disclosure relate to methods of determining the gastric cancer (GC) TME type of a subject having, suspected of having, or at risk of having GC. As used herein, a subject may be a mammal, for example a human, non-human primate, rodent (e.g., rat, mouse, guinea pig, etc.), dog, cat, horse etc. In some embodiments, the subject is a human. The terms “individual” or “subject” may be used interchangeably with “patient.” As used herein, “gastric cancer” or “GC” or “stomach cancer” refers to any gastric or gastrointestinal cancer, for example, gastric or gastrointestinal adenocarcinoma, or any other type of malignancy caused by one or more various genetic mutations in the body that affect cells (originally present in or metastasized to) the stomach and/or intestine of a subject. As used herein, “cancer” refers to any malignant and/or invasive growth or tumor caused by abnormal cell growth in a subject, including solid tumors, blood cancer, bone marrow or lymphoid cancer, etc. Examples of gastric cancers include but are not limited to esophageal cancer, stomach cancer, liver cancer, pancreatic cancer, colorectal cancer, anal cancer, adenocarcinoma of the stomach, fungating (polypoid) stomach cancer, ulcerating stomach cancer, superficial spreading stomach cancer, diffusely spreading stomach cancer, malignant lymphoma of the stomach, liposarcoma, fibrosarcoma, carcinosarcoma, and gastrointestinal stromal tumor (GST). A subject having GC may exhibit one or more signs or symptoms of GC, for example the presence of cancerous cells (e.g., tumor cells), fever, swelling, bleeding, nausea and vomiting, heartburn, and weight loss. In some embodiments, a subject having GC does not exhibit one or more signs or symptoms of GC. In some embodiments, a subject having GC has been diagnosed by a medical professional (e.g., a licensed physician) as having GC based upon one or more assays (e.g., clinical assays, molecular diagnostics, etc.) that indicate that the subject has GC, even in the absence of one or more signs or symptoms.

A subject suspected of having GC typically exhibits one or more signs or symptoms of GC. In some embodiments, a subject suspected of having GC exhibits one or more signs or symptoms of GC but has not been diagnosed by a medical professional (e.g., a licensed physician) and/or has not received a test result (e.g., a clinical assay, molecular diagnostic, etc.) indicating that the subject has GC.

A subject a risk of having GC may or may not exhibit one or more signs or symptoms of GC. In some embodiments, a subject at risk of having GC comprises one or more risk factors that increase the likelihood that the subject will develop GC. Examples of risk factors include the presence of pre-cancerous cells in a clinical sample, having one or more genetic mutations that predispose the subject to developing cancer (e.g., GC), taking one or more medications that increase the likelihood that the subject will develop cancer (e.g., GC), family history of GC, and the like.

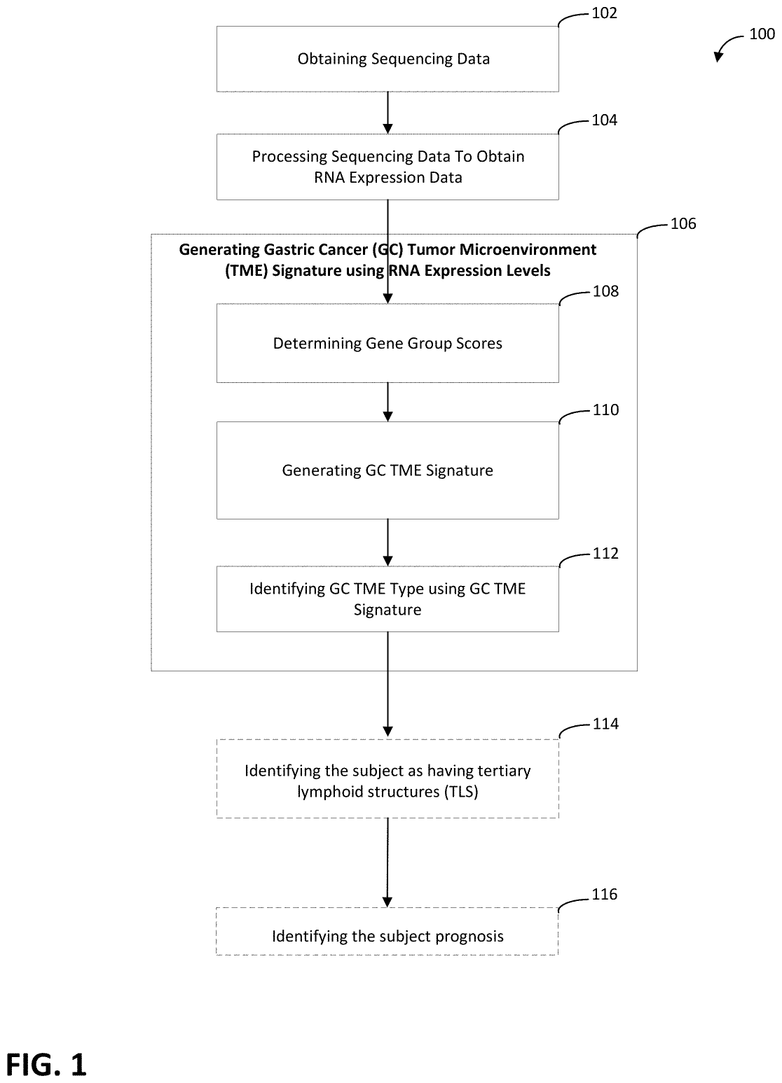

FIG. 1 is a flowchart of an illustrative process 100 for determining a GC TME signature for a subject, and using the determined GC TME signature to identify the GC TME type for the subject.

Various (e.g., some or all) acts of process 100 may be implemented using any suitable computing device(s). For example, in some embodiments, one or more acts of the illustrative process 100 may be implemented in a clinical or laboratory setting. For example, one or more acts of the process 100 may be implemented on a computing device that is located within the clinical or laboratory setting. In some embodiments, the computing device may directly obtain RNA expression data from a sequencing apparatus located within the clinical or laboratory setting. For example, a computing device included in the sequencing apparatus may directly obtain the RNA expression data from the sequencing apparatus. In some embodiments, the computing device may indirectly obtain RNA expression data from a sequencing apparatus that is located within or external to the clinical or laboratory setting. For example, a computing device that is located within the clinical or laboratory setting may obtain expression data via a communication network, such as Internet or any other suitable network, as aspects of the technology described herein are not limited to any particular communication network.

Additionally or alternatively, one or more acts of the illustrative process 100 may be implemented in a setting that is remote from a clinical or laboratory setting. For example, the one or more acts of process 100 may be implemented on a computing device that is located externally from a clinical or laboratory setting. In this case, the computing device may indirectly obtain RNA expression data that is generated using a sequencing apparatus located within or external to a clinical or laboratory setting. For example, the expression data may be provided to computing device via a communication network, such as Internet or any other suitable network. It should be appreciated that, in some embodiments, not all acts of process 100, as illustrated in FIG. 1, may be implemented using one or more computing devices. For example, the act 114 of identifying the subject as having tertiary lymphoid structures (TLS) may be implemented manually (e.g., by a clinician), automatically (e.g., by software identifying one or more TLS or a GC TME type associated with TLS), or in part manually and in part automatically (e.g., a clinician may identify TLS or a GC TME type associated with TLS in part using information generated by the software, for example, using the techniques described herein).

Process 100 begins at act 102 where sequencing data for a subject is obtained. In some embodiments, the sequencing data may be obtained by sequencing a biological sample (e.g., stomach biopsy and/or tumor tissue) obtained from the subject using any suitable sequencing technique. The sequencing data may include sequencing data of any suitable type, from any suitable source, and be in any suitable format. Examples of sequencing data, sources of sequencing data, and formats of sequencing data are described herein including in the section called “Obtaining RNA Expression Data”.

As one illustrative example, in some embodiments, the sequencing data may comprise bulk sequencing data. The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the sequencing data comprises microarray data.

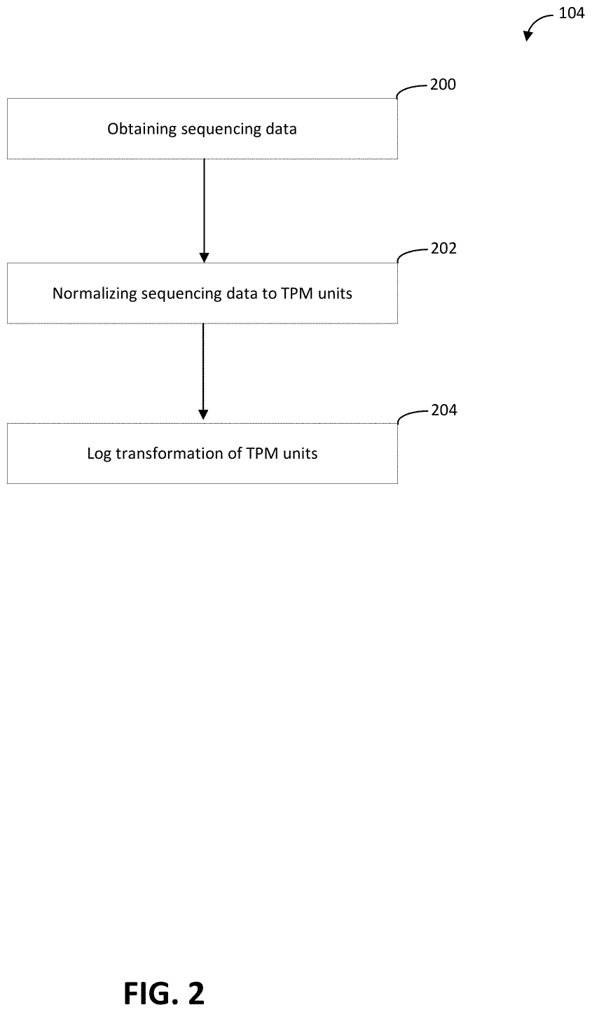

Next, process 100 proceeds to act 104, where the sequencing data obtained at act 102 is processed to obtain RNA expression data. This may be done in any suitable way and may involve normalizing bulk sequencing data to transcripts-per-million (TPM) units (or other units) and/or log transforming the RNA expression levels in TPM units. Converting the data to TPM units and normalization are described herein including with reference to FIG. 2.

Next, process 100 proceeds to act 106, where a gastric cancer (GC) tumor microenvironment (TME) signature is generated for the subject using the RNA expression data generated at act 104 (e.g., from bulk-sequencing data, converted to TPM units and subsequently log-normalized, as described herein including with reference to FIG. 2).

As described herein, in some embodiments, a GC TME signature comprises two or more (e.g., 2, 3, 4, 5, 6, 7, 8, 9, 10, 11, 12, 13, 14, 15, 16, 17, 18, 19, 20, 21, 22, 23, 24, 25, 26, 27, etc.) gene group scores. In some embodiments, the two or more gene group scores comprise gene group scores (which may also be referred to as gene group enrichment scores or gene group expression scores) for some or all of the gene groups shown in Table 1.

Accordingly, act 106 comprises: act 108 where the gene group scores are determined, act 110 where the GC TME signature is determined, and act 112 where the GC TME type is determined by using GC TME signature. In some embodiments, determining the gene group scores comprises determining, for each of multiple (e.g., some or all of the) gene groups listed in Table 1, a respective gene group score. In some embodiments, determining the gene group scores comprises determining respective gene group scores for 2, 3, 4, 5, 6, 7, 8, 9, 10, 11, 12, 13, 14, 15, 16, 17, 18, 19, or 20 gene groups (e.g., gene groups listed in Table 1). The gene group score for a particular gene group may be determined using RNA expression levels for at least some of the genes in the gene group (e.g., the RNA expression levels obtained at act 104). The RNA expression levels may be processed using a gene set enrichment analysis (GSEA) technique to determine the score for the particular gene group.

For example, in some embodiments, determining the GC TME signature comprises: determining gene group scores using RNA expression levels for at least three genes from each of at least two of the gene groups, the gene groups including: NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; T cells group: TBX21, ITK, CD3D, CD3E, CD3G, TRAC, TRBC1, TRBC2, CD28, CD5, TRAT1; B cells group: CD19, MS4A1, TNFRSF13C, CR2, TNFRSF17, TNFRSF13B, CD22, CD79A, CD79B, BLK, FCRL5, PAX5, STAP1; Treg group: FOXP3, CTLA4, IL10, TNFRSF18, CCR8, IKZF4, IKZF2; MDSC group: IDO1, ARG1, ILO, CYBB, PTGS2, IL4I1, IL6; Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; Proliferation rate group: MKI67, ESCO2, CETN3, CDK2, CCND1, CCNE1, AURKA, AURKB, E2F1, MYBL2, BUB1, CCNB1, MCM2, MCM6; and Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining the GC TME gene signature comprises: determining gene group scores using the RNA expression levels for at least three genes from each of at least two of the gene groups, the two gene groups including: (a) MHC I group: HLA-A, HLA-B, HLA-C, B2M, TAP1, TAP2, NLRC5, TAPBP; (b) MHC H group: HLA-DRA, HLA-DRB1, HLA-DMA, HLA-DPA1, HLA-DPB1, HLA-DMB, HLA-DQB1, HLA-DQA1, CIITA; and (c) Coactivation molecules group: CD28, CD40, TNFRSF4, ICOS, TNFRSF9, CD27, CD80, CD86, CD40LG, CD83, TNFSF4, ICOSLG, TNFSF9, CD70; (d) Effector cells group: IFNG, GZMA, GZMB, PRF1, GZMK, ZAP70, GNLY, FASLG, TBX21, EOMES, CD8A, CD8B; (e) T cell traffic group: CXCL9, CXCL10, CXCL11, CX3CL1, CCL3, CCL4, CX3CR1, CXCL16, CXCR6; (f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2DIB, NCR3; (g) T cells group: TBX21, ITK, CD3D, CD3E, CD3G, TRAC, TRBC1, TRBC2, CD28, CD5, TRAT1; (h) B cells group: CD19, MS4A1, TNFRSF13C, CR2, TNFRSF17, TNFRSF13B, CD22, CD79A, CD79B, BLK, FCRL5, PAX5, STAP1; (i) M1 signature group: NOS2, TNF, IL1B, SOCS3, CMKLR1, IRF5, IL12A, IL12B, IL23A; (j) Antitumor cytokines group: TNF, IFNB1, IFNA2, CCL3, TNFSF10, IL21; (k) Checkpoint inhibition group: PDCD1, CD274, CTLA4, LAG3, PDCD1LG2, BTLA, HAVCR2, TIGIT, VSIR; (l) Treg group: FOXP3, CTLA4, IL10, TNFRSF18, CCR8, IKZF4, IKZF2; (m) Neutrophil signature group: MPO, ELANE, PRTN3, CTSG, CXCR1, CXCR2, FCGR3B, CD177, FFAR2, PGLYRP1; (n) MDSC group: IDO1, ARG1, IL10, CYBB, PTGS2, IL4I1, IL6; (o) M2 signature group: IL10, MRC1, MSR1, CD163, CSF1R, IL4I1, SIGLEC1, CD68; (p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; (q) Angiogenesis group: VEGFA, VEGFB, VEGFC, PDGFC, CXCL8, CXCR2, FLT1, PGF, KDR, ANGPT1, ANGPT2, TEK, VWF, CDH5; (r) Proliferation rate group: MKI67, ESCO2, CETN3, CDK2, CCND1, CCNE1, AURKA, AURKB, E2F1, MYBL2, BUB1, CCNB1, MCM2, MCM6; and (s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

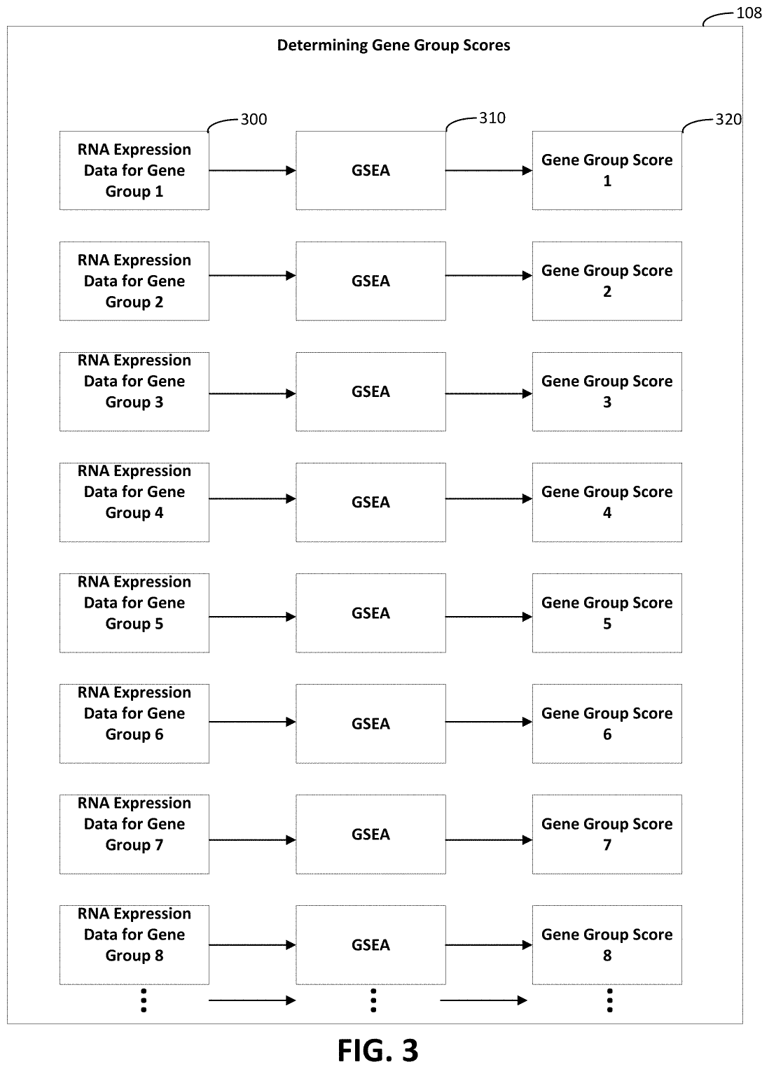

Aspects of determining the gene group scores are described herein, including with reference to FIG. 3 and in the Section titled “Gene Expression Signatures”.

As described above, at act 110, the GC TME signature is generated. In some embodiments, the GC TME signature consists of only gene group scores for one or more (e.g., all) gene groups listed in Table 1. In some embodiments, the GC TME signature comprises gene group scores for at least 2, 3, 4, 5, 6, 7, 8, 9, 10, 11, 12, 13, 14, 15, 16, 17, 18, 19, or 20 gene groups listed in Table 1. In some embodiments each gene group score is determined using RNA expression levels of some or all (e.g., at least 2, 3, 4, 5, 6, 7, 8, 9, 10, 11, 12, 13, 14, 15, 16, 17, 18, 19, 20, etc.) of the genes of each gene group listed in Table 1.

Next, process 100 proceeds to act 112, where an GC TME type is identified for the subject using the GC TME signature generated at act 110. This may be done in any suitable way. For example, in some embodiments, the each of the possible GC TME types is associated with a respective plurality of GC TME signature clusters. In such embodiments, a GC TME type for the subject may be identified by associating the GC TME signature of the subject with a particular one of the plurality of GC TME signature clusters; and identifying the GC TME type for the subject as the GC TME type corresponding to the particular one of the plurality of GC TME signature clusters to which the GC TME signature of the subject is associated. Examples of GC TME types are described herein. Aspects of identifying a GC TME type for a subject are described herein including in the section below titled “Generating GC TME Signature and Identifying TME Type”. In some embodiments, process 100 completes after act 112 completes.

In some such embodiments the determined GC TME signature and/or identified GC TME Type may be stored for subsequent use, provided to one or more recipients (e.g., a clinician, a researcher, etc.), and/or used to update the GC TME signature clusters (as described hereinbelow).

However, in some embodiments, one or more other acts are performed after act 112. For example, in the illustrated embodiment, a subject may be identified as having tertiary lymphoid structures (TLS) based on the GC TME type determined for the subject. For example, in some embodiments, the subject is identified (at act 114) as having a TLS when the subject is identified as having GC TME type E. Subsequently, or as an alternative to act 114, process 100 may proceed to act 116, where the subject's prognosis is identified using the GC TME type identified in act 112. For example, a subject may be identified as having an increased likelihood of having a good prognosis (e.g., as measured by overall survival (OS) or progression-free survival (PFS) when the subject is identified as having GC type E.

In some embodiments, when the subject is identified as having tertiary lymphoid structures, for example because GC TME type E is determined for the subject using a GC TME signature, an immunotherapy may be administered to the subject. In some embodiments, the immunotherapy comprises a PD1 inhibitor. In some embodiments, the PD1 inhibitor comprises pembrolizumab.

### Biological Samples

Aspects of the disclosure relate to methods for determining a GC TME type of a subject by obtaining sequencing data from a biological sample that has been obtained from the subject.

The biological sample may be from any source in the subject's body including, but not limited to, any fluid such as blood (e.g., whole blood, blood serum, or blood plasma), lymph node, stomach, small intestine. Other source in the subject's body may be from saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, bronchus, salivary gland, tongue, oral cavity, nasal cavity, vaginal cavity, anal cavity, bone, bone marrow, brain, thymus, spleen, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

The biological sample may be any type of sample including, for example, a sample of a bodily fluid, one or more cells, one or more pieces of tissue(s) or organ(s). In some embodiments, the biological sample comprises gastrointestinal tissue sample of the subject. Examples of gastrointestinal tissue samples include but are not limited to mucosal tissue, submucosal tissue, muscular layer tissue, and serous layer tissue (also referred to as serosa tissue). In some embodiments, a gastrointestinal tissue sample comprises one or more cell types derived from a stomach (e.g., mucous cells, parietal cells, chief cells, endocrine cells, etc.). In some embodiments, a gastrointestinal tissue sample comprises one or more cell types derived from gastrointestinal tissue, for example enterocytes, Paneth cells, goblet cells, neuroendocrine cells, etc.

In some embodiments, a gastrointestinal tissue sample may be obtained from a subject using a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

A sample of lymph node or blood, in some embodiments, refers to a sample comprising cells, e.g., cells from a blood sample or lymph node sample. In some embodiments, the sample comprises non-cancerous cells. In some embodiments, the sample comprises pre-cancerous cells. In some embodiments, the sample comprises cancerous cells. In some embodiments, the sample comprises blood cells. In some embodiments, the sample comprises lymph node cells. In some embodiments, the sample comprises lymph node cells and blood cells.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

In some embodiments, a sample of blood is collected to obtain the cell-free nucleic acid (e.g., cell-free DNA) in the blood.

In some embodiments, the sample may be from a cancerous tissue or an organ or a tissue or organ suspected of having one or more cancerous cells. In some embodiments, the sample may be from a healthy (e.g., non-cancerous) tissue or organ. In some embodiments, a sample from a subject (e.g., a biopsy from a subject) may include both healthy and cancerous cells and/or tissue. In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) samples may be taken from a subject for analysis. In some embodiments, one sample from a subject will be analyzed. In certain embodiments, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments, and may be taken from the same region or a different region. As a non-limiting example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor prior to and subsequent to a treatment).

Any of the biological samples described herein may be obtained from the subject using any known technique. See, for example, the following publications on collecting, processing, and storing biological samples, each of which is incorporated by reference herein in its entirety: Biospecimens and biorepositories: from afterthought to science by Vaught et al. (Cancer Epidemiol Biomarkers Prev. 2012 February; 21(2):253-5), and Biological sample collection, processing, storage and information management by Vaught and Henderson (IARC Sci Publ. 2011; (163):23-42).

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one form to another form such that the first form is no longer detected at the same level as before degradation.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilisation. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris-Cl; 0.5 mM EDTA, pH 9.0)) and other coagulants, and Acids Citrate Dextronse (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −8-° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

### Obtaining RNA Expression Data

Aspects of the disclosure relate to methods of determining a GC TME type of a subject using sequencing data or RNA expression data obtained from a biological sample from the subject.

The sequencing data may be obtained from the biological sample using any suitable sequencing technique and/or apparatus. In some embodiments, the sequencing apparatus used to sequence the biological sample may be selected from any suitable sequencing apparatus known in the art including, but not limited to, Illumina™, SOLid™, Ion Torrent™, PacBio™, a nanopore-based sequencing apparatus, a Sanger sequencing apparatus, or a 454™ sequencing apparatus. In some embodiments, sequencing apparatus used to sequence the biological sample is an Illumina sequencing (e.g., NovaSeq™, NextSeq™, HiSeq™, MiSeq™, or MiniSeq™) apparatus.

After the sequencing data is obtained, it is processed in order to obtain the RNA expression data. RNA expression data may be acquired using any method known in the art including, but not limited to: whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, RNA expression data may be obtained using a microarray assay.

In some embodiments, the sequencing data is processed to produce RNA expression data. In some embodiments, RNA sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Pill Melsted and Lior Pachter, Near-optimal probabilistic RNA-seq quantification, Nature Biotechnology 34, 525-527 (2016), doi:10.1038/nbt.3519, which is incorporated by reference in its entirety herein.

In some embodiments, microarray expression data is processed using a bioinformatics R package, such as “affy” or “limma”, in order to produce expression data. The “affy” software is described in Bioinformatics. 2004 Feb. 12; 20(3):307-15. doi: 10.1093/bioinformatics/btg405. “affy—analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law C W, Shi W, Smyth G K “limma powers differential expression analyses for RNA-sequencing and microarray studies.” Nucleic Acids Res. 2015 Apr. 20; 43 (7):e47. 20. https://doi.org/10.1093/nar/gkv007 PMID: 25605792, PMCID: PMC4402510, which is incorporated by reference herein its entirety.

In some embodiments, sequencing data and/or expression data comprises more than 5 kilobases (kb). In some embodiments, the size of the obtained RNA data is at least 10 kb. In some embodiments, the size of the obtained RNA sequencing data is at least 100 kb. In some embodiments, the size of the obtained RNA sequencing data is at least 500 kb. In some embodiments, the size of the obtained RNA sequencing data is at least 1 megabase (Mb). In some embodiments, the size of the obtained RNA sequencing data is at least 10 Mb. In some embodiments, the size of the obtained RNA sequencing data is at least 100 Mb. In some embodiments, the size of the obtained RNA sequencing data is at least 500 Mb. In some embodiments, the size of the obtained RNA sequencing data is at least 1 gigabase (Gb). In some embodiments, the size of the obtained RNA sequencing data is at least 10 Gb. In some embodiments, the size of the obtained RNA sequencing data is at least 100 Gb. In some embodiments, the size of the obtained RNA sequencing data is at least 500 Gb.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the expression data comprises next-generation sequencing (NGS) data. In some embodiments, the expression data comprises microarray data.

Expression data (e.g., indicating expression levels) for a plurality of genes may be used for any of the methods or compositions described herein. The number of genes which may be examined may be up to and inclusive of all the genes of the subject. In some embodiments, expression levels may be determined for all of the genes of a subject. As a non-limiting example, four or more, five or more, six or more, seven or more, eight or more, nine or more, ten or more, eleven or more, twelve or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 35 or more, 40 or more, 50 or more, 60 or more, 70 or more, 80 or more, 90 or more, 100 or more, 125 or more, 150 or more, 175 or more, 200 or more, 225 or more, 250 or more, 275 or more, or 300 or more genes may be used for any evaluation described herein. As another set of non-limiting examples, the expression data may include, for each gene group listed in Table 1, expression data for at least 5, at least 10, at least 15, at least 20, at least 25, at least 35, at least 50, at least 75, at least 100 genes selected from each gene group.

In some embodiments, RNA expression data is obtained by accessing the RNA expression data from at least one computer storage medium on which the RNA expression data is stored. Additionally or alternatively, in some embodiments, RNA expression data may be received from one or more sources via a communication network of any suitable type. For example, in some embodiment, the RNA expression data may be received from a server (e.g., a SFTP server, or Illumina BaseSpace).

The RNA expression data obtained may be in any suitable format, as aspects of the technology described herein are not limited in this respect. For example, in some embodiments, the RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

FIG. 2 shows an exemplary process 104 for processing sequencing data to obtain RNA expression data from sequencing data. Process 104 may be performed by any suitable computing device or devices, as aspects of the technology described herein are not limited in this respect.

For example, process 104 may be performed by a computing device part of a sequencing apparatus. In other embodiments, process 104 may be performed by one or more computing devices external to the sequencing apparatus.

Process 104 begins at act 200, where sequencing data is obtained from a biological sample obtained from a subject. The sequencing data is obtained by any suitable method, for example, using any of the methods described herein including in the Section titled “Biological Samples”.

In some embodiments, the sequencing data obtained at act 104 comprises RNA-seq data. In some embodiments, the biological sample comprises blood or tissue. In some embodiments, the biological sample comprises one or more tumor cells, for example, one or more GC tumor cells.

Next, process 104 proceeds to act 202 where the sequencing data obtained at act 200 is normalized to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the gcrma package. Aspects of the gcrma package are described in Wu J, Gentry RIwcfJMJ (2021). “gcrma: Background Adjustment Using Sequence Information. R package version 2.66.0.”, which is incorporated by reference in its entirety herein. In some embodiments, RNA expression level in TPM units for a particular gene may be calculated according to the following formula:

$$A \cdot \frac{1}{\sum ( A )} \cdot {10}^{6} \\ Where A = \frac{total reads mapped to gene \cdot {10}^{3}}{gene length in bp}$$

Next, process 104 proceeds to act 204, where the RNA expression levels in TPM units (as determined at act 202) may be log transformed. Process 104 is illustrative and there are variations. For example, in some embodiments, one or both of acts 202 and 204 may be omitted. Thus, in some embodiments, the RNA expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

RNA expression data obtained by process 104 can include the sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by process 104 can include information included in a FASTA file, a description and/or quality scores included in a FASTQ file, an aligned position included in a BAM file, and/or any other suitable information obtained from any suitable file.

### Gene Expression Signatures

Aspects of the disclosure relate to processing of expression data to determine one or more gene expression signatures (e.g., a GC TME signature). In some embodiments, expression data (e.g., RNA expression data) is processed using a computing device to determine the one or more gene expression signatures. In some embodiments, the computing device may be operated by a user such as a doctor, clinician, researcher, patient, or other individual. For example, the user may provide the expression data as input to the computing device (e.g., by uploading a file), and/or may provide user input specifying processing or other methods to be performed using the expression data.

In some embodiments, expression data may be processed by one or more software programs running on computing device.

In some embodiments, methods described herein comprise an act of determining a GC TME signature comprising gene group scores for respective gene groups in a plurality of gene groups. In some embodiments, a GC TME signature comprises gene group scores for at least one (e.g., 1, 2, 3, 4, 5, 6, 7, 8, 9, 10, 11, 12, 13, 14, 15, 16, 17, 18, 19, or 20) of the gene groups listed in Table 1.

The number of genes in a gene group used to determine a gene group score may vary. In some embodiments, all RNA expression levels for all genes in a particular gene group may be used to determine a gene group score for the particular gene group. In other embodiments, RNA expression data for fewer than all genes may be used (e.g., RNA expression levels for at least two genes, at least three genes, at least five genes, between 2 and 10 genes, between 5 and 15 genes, between 3 and 30 genes, or any other suitable range within these ranges).

In some embodiments, a GC TME signature comprises a gene group score for the MHC I gene group. In some embodiments, this gene group score may be calculated using RNA expression levels of at least three genes (e.g., at least three genes, at least four genes, at least five genes, at least six genes, or at least seven genes) in the MHC I gene group, which is defined by its constituent genes: HLA-A, HLA-B, HLA-C, B2M, TAP1, TAP2, NLRC5, TAPBP.

In some embodiments, a GC TME signature comprises a gene group score for the MHC II gene group. In some embodiments, this gene group score may be calculated using RNA expression levels of at least three genes (e.g., at least three genes, at least four genes, at least five genes, at least six genes, at least seven genes, at least eight genes) in the MHC II gene group, which is defined by its constituent gene: HLA-DRA, HLA-DRB1, HLA-DMA, HLA-DPA1, HLA-DPB1, HLA-DMB, HLA-DQB1, HLA-DQA1, CIITA.

In some embodiments, a GC TME signature comprises a gene group score for the Coactivation molecules group. In some embodiments, this gene group score may be calculated using RNA expression levels of at least three genes (e.g., at least three genes, at least four genes, at least five genes, at least six genes, at least seven genes, at least eight genes, at least nine genes, or at least ten genes) in the Coactivation molecules group, which is defined by its constituent genes: CD28, CD40, TNFRSF4, ICOS, TNFRSF9, CD27, CD80, CD86, CD40LG, CD83, TNFSF4, ICOSLG, TNFSF9, CD70.

In some embodiments, a GC TME signature comprises a gene group score for the Effector cells group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the Effector cells group, which is defined by its constituent genes: IFNG, GZMA, GZMB, PRF1, GZMK, ZAP70, GNLY, FASLG, TBX21, EOMES, CD8A, CD8B.

In some embodiments, a GC TME signature comprises a gene group score for the T cell traffic group. In some embodiments, this gene group score may be calculated using RNA expression levels of at least three genes (e.g., at least three genes, at least four genes, at least five genes, at least six genes, at least seven genes, or at least eight genes) in the T cell traffic group, which is defined by its constituent genes: CXCL9, CXCL10, CXCL11, CX3CL1, CCL3, CCL4, CX3CR1, CXCL16, CXCR6.

In some embodiments, a GC TME signature comprises a gene group score for the NK cells group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the NK cells group, which is defined by its constituent genes: NKG7, CD160, CD244, NCR1, KLRC2, KLRK1, CD226, GZMH, GNLY, IFNG, KIR2DL4, EOMES, GZMB, FGFBP2, KLRF1, SH2DIB, NCR3.

In some embodiments, a GC TME signature comprises a gene group score for the T cells group. In some embodiments, this gene group score may be calculated using RNA expression levels of at least three genes (e.g., at least three genes, at least four genes, at least five genes, at least six genes, at least seven genes, at least eight genes, at least nine genes, or at least ten genes) in the T cells group, which is defined by its constituent genes: MKI67, ESCO2, CETN3, CDK2, CCND1, CCNE1, AURKA, AURKB, E2F1, MYBL2, BUB1, PLK1, CCNB1, MCM2, and MCM6.

In some embodiments, a GC TME signature comprises a gene group score for the B cells group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the B cells group, which is defined by its constituent genes: CD19, MS4A1, TNFRSF13C, CR2, TNFRSF17, TNFRSF13B, CD22, CD79A, CD79B, BLK, FCRL5, PAX5, STAP1.

In some embodiments, a GC TME signature comprises a gene group score for the M1 signature group. In some embodiments, this gene group score may be calculated using RNA expression levels of at least three genes (e.g., at least three genes, at least four genes, at least five genes, at least six genes, at least seven genes, or at least eight genes) in the M1 signature group, which is defined by its constituent genes: NOS2, TNF, IL1B, SOCS3, CMKLR1, IRF5, IL12A, IL12B, IL23A.

In some embodiments, a GC TME signature comprises a gene group score for the Antitumor cytokines group. In some embodiments, this gene group score may be calculated using RNA expression levels of at least three genes (e.g., at least three genes, at least four genes, or at least five genes) in the Antitumor cytokines group, which is defined by its constituent genes: TNF, IFNB1, IFNA2, CCL3, TNFSF10, IL21.

In some embodiments, a GC TME signature comprises a gene group score for the Checkpoint inhibition group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the Checkpoint inhibition group, which is defined by its constituent genes: PDCD1, CD274, CTLA4, LAG3, PDCD1LG2, BTLA, HAVCR2, TIGIT, VSIR.

In some embodiments, a GC TME signature comprises a gene group score for the Treg group. In some embodiments, this gene group score may be calculated using RNA expression levels of at least three genes (e.g., at least three genes, at least four genes, at least five genes, or at least six genes) in the Treg group, which is defined by its constituent genes: FOXP3, CTLA4, IL10, TNFRSF18, CCR8, IKZF4, IKZF2.

In some embodiments, a GC TME signature comprises a gene group score for the Neutrophil signature group. In some embodiments, this gene group score may be calculated using RNA expression levels of at least three genes (e.g., at least three genes, at least four genes, at least five genes, at least six genes, at least seven genes, at least eight genes, or at least nine genes) in the Neutrophil group, which is defined by its constituent genes: MPO, ELANE, PRTN3, CTSG, CXCR1, CXCR2, FCGR3B, CD177, FFAR2, PGLYRP1.

In some embodiments, a GC TME signature comprises a gene group score for the MDSC group. In some embodiments, this gene group score may be calculated using RNA expression levels of at least three genes (e.g., at least three genes, at least four genes, at least five genes, or at least six genes) in the MDSC group, which is defined by its constituent genes: IDO1, ARG1, IL10, CYBB, PTGS2, IL4I1, IL6.

In some embodiments, a GC TME signature comprises a gene group score for the M2 signature group. In some embodiments, this gene group score may be calculated using RNA expression levels of at least three genes (e.g., at least three genes, at least four genes, at least five genes, at least six genes, or at least seven genes) in the M2 group, which is defined by its constituent genes: IL10, MRC1, MSR1, CD163, CSF1R, IL4I1, SIGLEC1, CD68.

In some embodiments, a GC TME signature comprises a gene group score for the Cancer associated fibroblast (CAF) group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the Cancer associated fibroblast (CAF) group, which is defined by its constituent genes: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX.

In some embodiments, a GC TME signature comprises a gene group score for the angiogenesis group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the angiogenesis group, which is defined by its constituent genes: VEGFA, VEGFB, VEGFC, PDGFC, CXCL8, CXCR2, FLT1, PGF, KDR, ANGPT1, ANGPT2, TEK, VWF, CDH5.

In some embodiments, a GC TME signature comprises a gene group score for the Proliferation rate group. In some embodiments, this gene group score may be calculated using RNA expression levels of at least three genes (e.g., at least three genes, at least four genes, at least five genes, or at least six genes) in the Proliferation rate group, which is defined by its constituent genes: MKI67, ESCO2, CETN3, CDK2, CCND1, CCNE1, AURKA, AURKB, E2F1, MYBL2, BUB1, CCNB1, MCM2, MCM6.

In some embodiments, a GC TME signature comprises a gene group score for the Lgr5 ISC group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the Lgr5_ISC group, which is defined by its constituent genes: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

In some embodiments, determining a GC TME signature comprises determining a respective gene group score for each of at least two of the following gene groups, using, for a particular gene group, RNA expression levels for at least three genes in the particular gene group to determine the gene group score for the particular group, the gene groups including: MHC I group: HLA-A, HLA-B, HLA-C, B2M, TAP1, TAP2, NLRC5, TAPBP; MHC II group: HLA-DRA, HLA-DRB1, HLA-DMA, HLA-DPA1, HLA-DPB1, HLA-DMB, HLA-DQB1, HLA-DQA1, CIITA; Coactivation molecules group: CD28, CD40, TNFRSF4, ICOS, TNFRSF9, CD27, CD80, CD86, CD40LG, CD83, TNFSF4, ICOSLG, TNFSF9, CD70; Effector cells group: IFNG, GZMA, GZMB, PRF1, GZMK, ZAP70, GNLY, FASLG, TBX21, EOMES, CD8A, CD8B; T cell traffic group: CXCL9, CXCL10, CXCL11, CX3CL1, CCL3, CCL4, CX3CR1, CXCL16, CXCR6; NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3; T cells group: TBX21, ITK, CD3D, CD3E, CD3G, TRAC, TRBC1, TRBC2, CD28, CD5, TRAT1; B cells group: CD19, MS4A1, TNFRSF13C, CR2, TNFRSF17, TNFRSF13B, CD22, CD79A, CD79B, BLK, FCRL5, PAX5, STAP1; M1 signature group: NOS2, TNF, IL1B, SOCS3, CMKLR1, IRF5, IL12A, IL12B, IL23A; Antitumor cytokines group: TNF, IFNB, IFNA2, CCL3, TNFSF10, IL21; Checkpoint inhibition group: PDCD1, CD274, CTLA4, LAG3, PDCD1LG2, BTLA, HAVCR2, TIGIT, VSIR; Treg group: FOXP3, CTLA4, IL10, TNFRSF18, CCR8, IKZF4, IKZF2; Neutrophil signature group: MPO, ELANE, PRTN3, CTSG, CXCR1, CXCR2, FCGR3B, CD177, FFAR2, PGLYRP1; MDSC group: IDO1, ARG1, IL10, CYBB, PTGS2, IL4I1, IL6; M2 signature group: IL10, MRC1, MSR1, CD163, CSF1R, IL4I1, SIGLEC1, CD68; Cancer associated fibroblast (CAF) group: LGALS1, COL1A1, COL1A2, COL5A1, ACTA2, FAP, LRP1, CD248, COL6A1, COL6A2, 5 COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX; Angiogenesis group: VEGFA, VEGFB, VEGFC, PDGFC, CXCL8, CXCR2, FLT1, PGF, KDR, ANGPT1, ANGPT2, TEK, VWF, CDH5; Proliferation rate group: MKI67, ESCO2, CETN3, CDK2, CCND1, CCNE1, AURKA, AURKB, E2F1, MYBL2, BUB1, CCNB1, MCM2, MCM6; and Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

A list of gene groups is provided in Table 1 below:

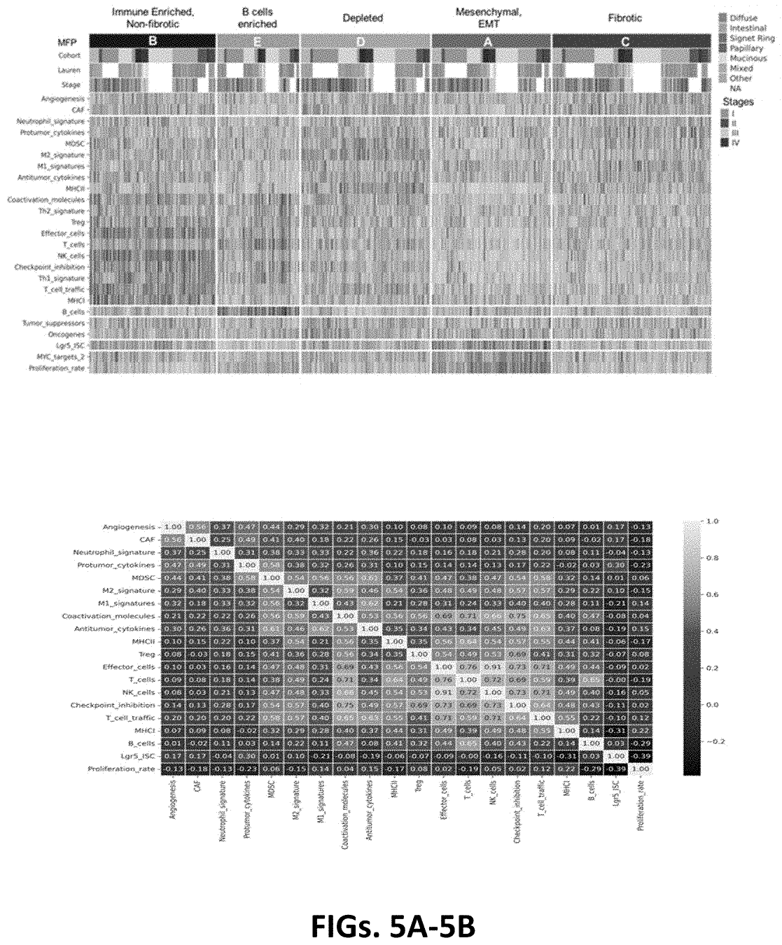

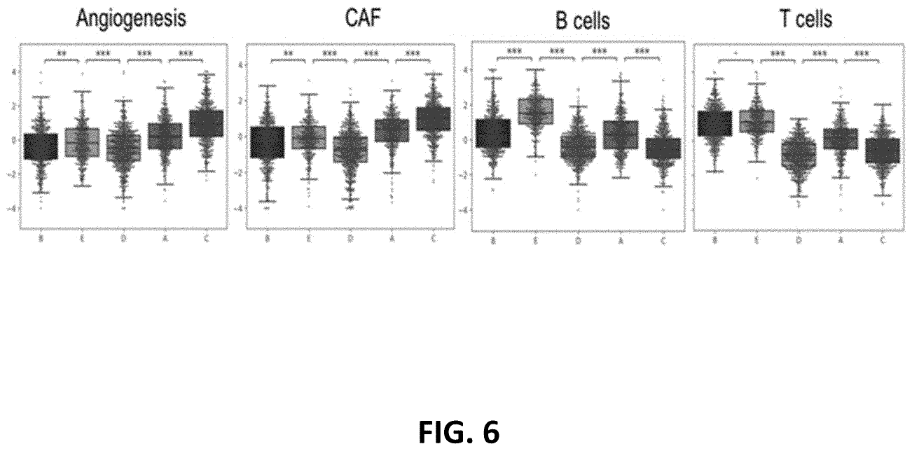

TABLE 1
List of Gene Groups, the left column providing the name of the Gene Group and the right column
providing examples of genes in the Gene Group. In some embodiments, a GC TME signature may
include scores for two or more of the gene groups in this table. In some embodiments, a GC TME
signature may include scores for the gene groups in this table that are denoted by bold text.
It should be noted that the names of the gene groups shown in Table 1 appear in some of the
figures (e.g., FIGS. 5A, 5B, 7, and 8A) with “_” instead of spaces due to how the graphics
were generated. For example, “T cells” appears as “T_cells” in those figures. Also
“M1 signature” is labeled “M1_signatures” in the figures.

| Gene Group Name | Constituent Genes |
|---|---|
| MHC I | HLA-A, HLA-B, HLA-C, B2M, TAP1, TAP2, NLRC5, TAPBP |
| MHC II | HLA-DRA, HLA-DRB1, HLA-DMA, HLA-DPA1, HLA-DPB1, HLA- |
| | DMB, HLA-DQB1, HLA-DQA1, CIITA |
| Coactivation molecules | CD28, CD40, TNFRSF4, ICOS, TNFRSF9, CD27, CD80, CD86, |
| | CD40LG, CD83, TNFSF4, ICOSLG, TNFSF9, CD70 |
| Effector cells | IFNG, GZMA, GZMB, PRF1, GZMK, ZAP70, GNLY, FASLG, TBX21, |
| | EOMES, CD8A, CD8B |
| T cell traffic | CXCL9, CXCL10, CXCL11, CX3CL1, CCL3, CCL4, CX3CR1, |
| | CXCL16, CXCR6 |
| NK cells | NKG7, CD160, CD244, NCR1, KLRC2, KLRK1, CD226, GZMH, |
| | GNLY, IFNG, KIR2DL4, EOMES, GZMB, FGFBP2, KLRF1, SH2D1B, |
| | NCR3 |
| T cells | TBX21, ITK, CD3D, CD3E, CD3G, TRAC, TRBC1, TRBC2, CD28, |
| | CD5, TRAT1 |
| B cells | CD19, MS4A1, TNFRSF13C, CR2, TNFRSF17, TNFRSF13B, CD22, |
| | CD79A, CD79B, BLK, FCRL5, PAX5, STAP1 |
| M1 signature | NOS2, TNF, IL1B, SOCS3, CMKLR1, IRF5, IL12A, IL12B, IL23A |
| Antitumor cytokines | TNF, IFNB1, IFNA2, CCL3, TNFSF10, IL21 |
| Checkpoint | PDCD1, CD274, CTLA4, LAG3, PDCD1LG2, BTLA, HAVCR2, TIGIT, |
| inhibition | VSIR |
| Treg | FOXP3, CTLA4, IL10, TNFRSF18, CCR8, IKZF4, IKZF2 |
| Neutrophil | MPO, ELANE, PRTN3, CTSG, CXCR1, CXCR2, FCGR3B, CD177, |
| signature | FFAR2, PGLYRP1 |
| MDSC | IDO1, ARG1, IL10, CYBB, PTGS2, IL4I1, IL6 |
| M2 signature | IL10, MRC1, MSR1, CD163, CSF1R, IL4I1, SIGLEC1, CD68 |
| Protumor cytokines | IL10, TGFB1, TGFB2, TGFB3, IL22, MIF, IL6 |
| CAF | LGALS1, COL1A1, COL1A2, COL5A1, ACTA2, FAP, LRP1, CD248, |
| | COL6A1, COL6A2, COL6A3, COL11A1, CXCL12, FBLN1, LUM, |
| | MFAP5, MMP3, MMP2, PDGFRB, PDGFRA, FN1, COL1A1, COL1A2, |
| | COL4A1, COL3A1, VTN, LGALS7, LGALS9, LAMA3, LAMB3, |
| | LAMC2, TNC, ELN, COL5A1, COL11A1, LGALS3, CA9, MMP9, |
| | MMP2, MMP1, MMP3, MMP12, MMP7, MMP11, PLOD2, ADAMTS4, |
| | ADAMTS5, LOX |
| Angiogenesis | VEGFA, VEGFB, VEGFC, PDGFC, CXCL8, CXCR2, FLT1, PGF, |
| | CXCL5, KDR, ANGPT1, ANGPT2, TEK, VWF, CDH5 |
| Proliferation rate | MKI67, ESCO2, CETN3, CDK2, CCND1, CCNE1, AURKA, AURKB, |
| | E2F1, MYBL2, BUB1, PLK1, CCNB1, MCM2, MCM6 |
| (Leucine-rich repeat- | ABTB2, AFAP1L1, APCDD1, ARHGEF4, ARNT2, AXIN2, BCL2, |
| containing G-protein | BEX1, BEX2, CAP2, CCDC46, CYP2E1, DGKG, DLGAP1, DTL, |
| coupled receptor 5) | DYNC2H1, EPHA4, FAM64A, FGFR4, FMNL2, FSTL1, GRAMD1A, |
| Lgr5 ISC | GRK4, IGF1R, IGFBP4, IL17RD, KIF12, KIF26B, KLHL13, LDHB, |
| | LGR5, LIFR, LOC285141, MDFIC, MPP3, NPNT, PITPNC1, PLP1, |
| | RASSF4, RNF157, SCN2B, SEPT6, SERTAD4, SLC1A2, SLC38A4, |
| | SLCO3A1, SLIT2, SOAT1, SORBS2, SOX4, TACC1, TMEM182, |
| | TNFRSF19, UTRN, ZNF141, ZNF273, ZNF493, ZNF626, ZNF678, |
| | ZNF680, ZNF714, ZNF85, ZNF92, ZNF93 |

As described above, aspects of the disclosure relate to determining an GC TME signature for a subject. That signature may include gene group scores (e.g., gene group scores generated using RNA expression data for gene groups listed in Table 1). Aspects of determining of these GC TME signatures is described next with reference to FIG. 3.

In some embodiments, a GC TME signature comprises gene group scores generated using a gene set enrichment analysis (GSEA) technique to determine a gene group score for one or more (e.g., 1, 2, 3, 4, 5, 6, 7, 8, 9, 10, 11, 12, 13, 14, 15, 16, 17, 18, 19, or 20) gene groups listed in Table 1. In some embodiments, a GC TME signature comprises gene group scores generated using a gene set enrichment analysis (GSEA) technique to determine a gene enrichment score for eight or more (e.g., 8, 9, 10, 11, 12, 13, 14, 15, 16, 17, 18, 19, or 20) gene groups listed in Table 1. In some embodiments, each gene group score is generated using a gene set enrichment analysis (GSEA) technique, using RNA expression levels of at least some genes in the gene group. In some embodiments, using a GSEA technique comprises using single-sample GSEA. Aspects of single sample GSEA (ssGSEA) are described in Barbie et al. Nature. 2009 Nov. 5; 462(7269): 108-112, the entire contents of which are incorporated by reference herein. In some embodiments, ssGSEA is performed according to the following formula:

$$ssGSEA score = \frac{{\sum }_{i}^{N} {r}_{i}^{1.25}}{{{\sum }^{N}}_{i} {r}_{i}^{0.25}} - \frac{( M - N + 1 )}{2}$$

where rᵢ represents the rank of the ith gene in expression matrix, where N represents the number of genes in the gene set (e.g., the number of genes in the first gene group when ssGSEA is being used to determine a gene group score for the first gene group using expression levels of the genes in the first gene group), and where M represents total number of genes in expression matrix. Additional, suitable techniques of performing GSEA are known in the art and are contemplated for use in the methods described herein without limitation. In some embodiments, a GC TME signature is calculated by performing ssGSEA on expression data from a plurality of subjects, for example expression data from one or more cohorts of subjects, such as GSE15459, GSE34942, GSE26253, GSE62254, GSE13861, GSE26901, GSE29272, GSE84437, GSE26899, GSE28541, GSE113255, PRJEB25780, SRP219269, and TCGA—stomach samples (e.g., STAD TCGA project), in order to produce a plurality of enrichment scores.

FIG. 3 depicts an illustrative example of how gene group scores may be determined as part of act 108 of process 100. As shown in the example of FIG. 3, a “GC TME signature” comprises multiple gene group scores 320 determined for respective multiple gene groups. Each gene group score, for a particular gene group, is computed by performing GSEA 310 (e.g., using ssGSEA) on RNA expression data for one or more (e.g., at least two, at least three, at least four, at least five, at least six, etc., or all) genes in the particular gene group 300.

For example, as shown in FIG. 3, a gene group score (labelled “Gene Group Score 1”) for gene group 1 (e.g., the Treg group) is computed from RNA expression data for one or more genes in gene group 1. As another example, a gene group score (labelled “Gene Group Score 2”) for gene group 2 (e.g., the T cells group) is computed from RNA expression data for one or more genes in gene group 2. As another example, a gene group score (labelled “Gene Group Score 3”) for gene group 3 (e.g., the NK cells group) is computed from RNA expression data for one or more genes in gene group 3. As another example, a gene group score (labelled “Gene Group Score 4”) for gene group 4 (e.g., the B cells group) is computed from RNA expression data for one or more genes in gene group 4. As another example, a gene group score (labelled “Gene Group Score 5”) for gene group 5 (e.g., the MDSC group) is computed from RNA expression data for one or more genes in gene group 5. As another example, a gene group score (labelled “Gene Group Score 6”) for gene group 6 (e.g., the CAF group) is computed from RNA expression data for one or more genes in gene group 6. As another example, a gene group score (labelled “Gene Group Score 7”) for gene group 7 (e.g., the Proliferation rate group) is computed from RNA expression data for one or more genes in gene group 7. As another example, a gene group score (labelled “Gene Group Score 8”) for gene group 8 (e.g., the Lgr5 ISC group) is computed from RNA expression data for one or more genes in gene group 8.

Although the example of FIG. 3 shows that the gene expression group expression score includes eight gene group scores for a respective set of eight gene groups, it should be appreciated that in other embodiments, the first gene expression signature may include scores for any suitable number of groups (e.g., not just 8; the number of groups could be fewer or greater than 8). As indicated by the vertical ellipsis in FIG. 3, determining gene group scores of a GC TME signature may comprise determining gene group scores for 9, 10, 11, 12, 13, 14, 15, 16, 17, 18, 19, 20, or more gene groups using RNA expression data from one or more respective genes in each respective gene group, as aspects of the technology described herein are not limited in this respect. In another example, a GC TME signature may include scores for only a subset of the gene groups listed in Table 1 above. As another example, the gene expression group score may include one or more scores for one or more gene groups other than those gene groups listed in Table 1 (either in addition to the score(s) for the groups in Table 1 or instead of one or more of the scores for the groups in Table 1).

In some embodiments, RNA expression levels for a particular gene group may be embodied in at least one data structure having fields storing the expression levels. The data structure or data structures may be provided as input to software comprising code that implements a GSEA technique (e.g., the ssGSEA technique) and processes the expression levels in the at least one data structure to compute a score for the particular gene group.

The number of genes in a gene group used to determine a gene group expression score may vary. In some embodiments, all RNA expression levels for all genes in a particular gene group may be used to determine a gene group score for the particular gene group. In other embodiments, RNA expression data for fewer than all genes may be used (e.g., RNA expression levels for at least two genes, at least three genes, at least five genes, between 2 and 10 genes, between 5 and 15 genes, or any other suitable range within these ranges).

In some embodiments, RNA expression levels for a particular gene group may be embodied in at least one data structure having fields storing the expression levels. The data structure or data structures may be provided as input to software comprising code that is configured to perform suitable scaling (e.g., median scaling) to produce a score for the particular gene group.

In some embodiments, ssGSEA is performed on expression data comprising three or more (e.g., 3, 4, 5, 6, 7, 8, 9, 10, 11, 12, 13, 14, 15, 16, 17, 18, 19, or 20) gene groups set forth in Table 1. In some embodiments, each of the gene groups separately comprises one or more (e.g., 1, 2, 3,4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or more) genes listed in Table 1. In some embodiments, a GC TME signature is produced by performing ssGSEA on all 20 of the gene groups in Table 1, each gene group including all listed genes in Table 1.

In some embodiments, one or more (e.g., a plurality) of enrichment scores are normalized in order to produce a GC TME signature for the expression data (e.g., expression data of the subject or of a cohort of subjects). In some embodiments, the enrichment scores are normalized by median scaling. In some embodiments, the enrichment scores are normalized by rank estimation and median scaling. In some embodiments, median scaling comprises clipping the range of enrichment scores, for example clipping to about −1.0 to about +1.0, −2.0 to about +3.0, −3.0 to about +3.0, −4.0 to +4.0, −5.0 to about +5.0.

In some embodiments, a GC TME signature of a subject processed using a clustering algorithm to identify a GC tumor microenvironment type (e.g. a GC TME type). In some embodiments, the clustering comprises unsupervised clustering. In some embodiments, the unsupervised clustering comprises a dense clustering approach. In some embodiments, the unsupervised clustering comprises a hierarchical clustering approach. In some embodiments, clustering comprises calculating intersample similarity (e.g., using a Pearson correlation coefficient that, for example, may take on values in the range of [−1,1]), converting the distance matrix into a graph where each sample forms a node and two nodes form an edge with a weight equal to their Pearson correlation coefficient, removing edges with weight lower than a specified threshold, and applying a Louvain community detection algorithm to calculate graph partitioning into clusters. In some embodiments, the optimum weight threshold for observed clusters was calculated by employing minimum DaviesBouldin, maximum Calinski-Harabasz, and Silhouette techniques. In some embodiments, separations with low-populated clusters (<5% of samples) are excluded.

In some embodiments, a GC TME signature of a subject is compared to pre-existing clusters of GC TME types and assigned a GC TME type based on that comparison.

Some aspects of determining gene group scores for gene groups are also described in U.S. Patent Publication No. 2020-0273543, entitled “SYSTEMS AND METHODS FOR GENERATING, VISUALIZING AND CLASSIFYING MOLECULAR FUNCTIONAL PROFILES”, the entire contents of which are incorporated by reference herein.

### Generating GC TME Signature and Identifying TME Type

As described herein, FIG. 1 illustrates the determination of a subject's GC TME signature and, optionally, identification of the subject's prognosis using the identified GC TME signature.

As described herein, in some embodiments, one of a plurality of different GC TME types may be identified for the subject using the GC TME signature determined for the subject using the techniques described herein. In some embodiments, the GC TME type comprises GC TME type A, GC TME type B, GC TME type C, GC TME type D, and GC TME type E, as described herein and further below. In some embodiments, each of the plurality of GC TME types is associated with a respective GC TME signature cluster in a plurality of GC TME signature clusters. The GC TME type for a subject may be determined by: (1) associating the GC TME signature of the subject with a particular one of the plurality of GC TME signature clusters; and (2) identifying the GC TME type for the subject as the GC TME type corresponding to the particular one of the plurality of GC TME signature clusters to which the GC TME signature of the subject is associated.

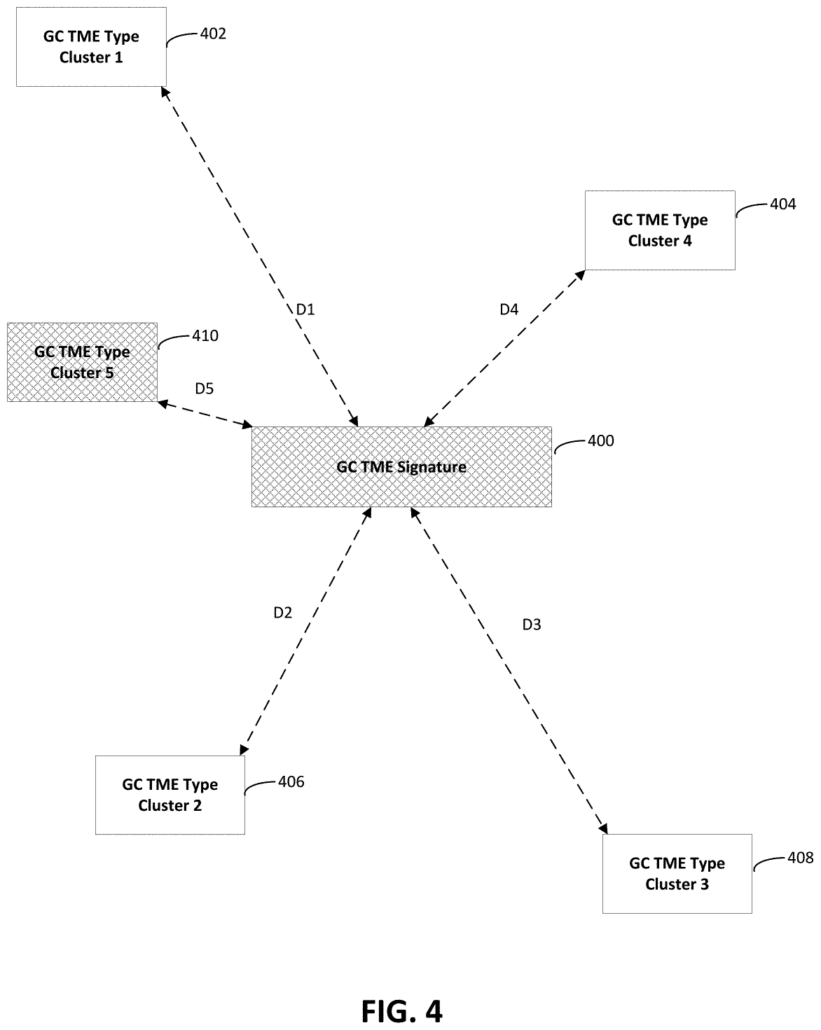

FIG. 4 shows an illustrative GC TME signature 400. In some embodiments, the GC TME signature comprises at least eight gene group scores for the following gene groups: NK cell group, T cell group, B cell group, Treg cells group, MDSC group, CAF group, Proliferation rate group, and the Lgr5 ISC group. However, it should be appreciated, that a GC TME signature may include fewer scores than the number of scores shown in FIG. 4 (e.g., by omitting scores for one or more of the gene groups listed in Table 1) or more scores than the number of scores shown in FIG. 4 (e.g., by including scores for one or more other gene groups in addition to or instead of the gene groups listed in Table 1). In some embodiments, a GC TME signature may be embodied in at least one data structure comprising fields storing the gene group scores part of the GC TME signature.

In some embodiments, the GC TME signature clusters may be generated by: (1) obtaining GC TME signatures (using the techniques described herein) for a plurality of subjects; and (2) clustering the GC TME signatures so obtained into the plurality of clusters. Any suitable clustering technique may be used for this purpose including, but not limited to, a dense clustering algorithm, spectral clustering algorithm, k-means clustering algorithm, hierarchical clustering algorithm, and/or an agglomerative clustering algorithm.

For example, intersample similarity may be calculated using a Pearson correlation. A distance matrix may be converted into a graph where each sample forms a node and two nodes form an edge with a weight equal to their Pearson correlation coefficient. Edges with weight lower than a specified threshold may be removed. A Louvain community detection algorithm may be applied to calculate graph partitioning into clusters. To mathematically determine the optimum weight threshold for observed clusters minimum DaviesBouldin, maximum Calinski-Harabasz, and Silhouette techniques may be employed. Separations with low-populated clusters (<5% of samples) may be excluded.

Accordingly, in some embodiments, generating the GC TME signature clusters involves: (A) obtaining multiple sets of RNA expression data obtained by sequencing biological samples from multiple respective subjects, each of the multiple sets of RNA expression data indicating RNA expression levels for genes in a first plurality of gene groups (e.g., one or more of the gene groups in Table 1); (B) generating multiple GC TME signatures from the multiple sets of RNA expression data, each of the multiple GC TME signatures comprising gene group scores for respective gene groups, the generating comprising, for each particular one of the multiple TME signatures: (i) determining the GC TME signature by determining the gene group scores using the RNA expression levels in the particular set of RNA expression data for which the particular one GC TME signature is being generated, and (ii) clustering the multiple GC signatures to obtain the plurality of GC TME signature clusters.

The resulting GC TME signature clusters may each contain any suitable number of GC TME signatures (e.g., at least 10, at least 100, at least 500, at least 500, at least 1000, at least 5000, between 100 and 10,000, between 500 and 20,000, or any other suitable range within these ranges), as aspects of the technology described herein are not limited in this respect.

The number of GC TME signature clusters in this example is five. And although, in some embodiments, it may be possible that the number of clusters is different, it should be appreciated that an important aspect of the present disclosure is the inventors' discovery that GC may be characterized into five types based upon the generation of GC TME signatures using methods described herein.

For example, as shown in FIG. 4, a subject's GC TME signature 400 may be associated with one of five GC TME clusters: 402, 404, 406, 408, and 410. Each of the clusters 402, 404, 406, 408 and 410 may be associated with respective GC TME type. In this example, the GC TME signature 400 is compared to each cluster (e.g., using a distance-based comparison or any other suitable metric) and, based on the result of the comparison, the GC TME signature 400 is associated with the closest GC signature cluster (when a distance-based comparison is performed, or the “closest” in the sense of whatever metric or measure of distance is used). In this example, GC TME signature 400 is associated with GC TME Type Cluster 5 410 (as shown by the consistent shading) because the measure of distance D5 between the GC TME signature 400 and (e.g., a centroid or other point representative of) cluster 410 is smaller than the measures of the distance D1, D2, D3, and D4 between the GC TME signature 400 and (e.g., a centroid or other point(s) representative of) clusters 402, 404, 406, and 408, respectively.

In some embodiments, a subject's GC TME signature may be associated with one of five GC TME signature clusters by using a machine learning technique (e.g., such as k-nearest neighbors (KNN) or any other suitable classifier) to assign the GC TME signature to one of the four GC TME signature clusters. The machine learning technique may be trained to assign GC TME signatures on the meta-cohorts represented by the signatures in the clusters.

In some embodiments, GC TME types include GC TME type A, GC TME type B, GC TME type C, GC TME type D, and GC TME type E. The GC TME types described herein may be described by qualitative characteristics, for example high signals for certain gene expression signatures or scores or low signals for certain other gene expression signatures or scores. In some embodiments, a “high” signal refers to a gene expression signal or score (e.g., an enrichment score) that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more increased relative to the score of the same gene or gene group in a subject having a different type of GC. In some embodiments, a “low” signal refers to a gene expression signal or score (e.g., an enrichment score) that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more decreased relative to the score of the same gene or gene group in a subject having a different type of GC TME.

Without wishing to be bound by any theory, the tumor microenvironment of GC may contain variable numbers of immune cells, stromal cells, blood vessels and extracellular matrix. In some embodiments, “GC TME type A” is characterized by “Mesenchymal, EMT” type. In some embodiments, GC TME type A (mesenchymal) type is characterized by having the highest LGR5+ stem cell signature relative to any other GC TME types. In some embodiments, GC TME type A GC subjects also have high stromal compartment gene group signatures, for example cancer-associated fibroblasts (CAF) and angiogenesis, relative to other GC TME types. In some embodiments, GC TME type A subject are characterized by high levels of protumor cytokines and medium immune compartment signatures. In some embodiments, GC TME type A subjects have low tumor proliferation rates (relative to other GC types). In some embodiments, GC TME type A tumors are diffuse (by e.g. as assessed by Lauren classification).

GC TME type A has been observed, in some embodiments, to be the most aggressive GC TME type, and subjects having GC TME type A have been observed in some embodiments to have worse prognosis (e.g., by overall survival (OS)) relative to other GC types.

In some embodiments, “GC TME type B” is also referred to as “Immune-enriched, non-fibrotic (IE)” type. In some embodiments, GC TME type B (IE) is characterized by high levels of immune-active infiltrate with a significant number of effector cells and NK cells relative to other GC TME types. In some embodiments, GC TME type B subjects have the highest tumor mutational burden (TMB). In some embodiments, subjects having GC TME type B have been observed to have better prognosis (e.g., by overall survival (OS)) relative to other GC TME types.

In some embodiments, “GC TME type C” is also referred to as “Fibrotic” or “immune non-inflamed” type. In some embodiments, GC TME type C is characterized by a highly fibrotic tumor microenvironment with dense collagen formation. In some embodiments, GC TME type C is characterized by minimal lymphocyte infiltration with elevated angiogenesis, relative to other GC TME types. In some embodiments, Cancer-associated fibroblasts (CAFs) signatures are abundant in GC TME type C. In some embodiments, GC TME type C is characterized by a high level of both protumor and antitumor cytokines. In some embodiments, subjects having GC TME type C have a poorer prognosis (e.g., as measured by OS) than subjects having other GC TME types.

In some embodiments, GC TME type D is also referred to as “Immune desert” type. In some embodiments, GC TME type D is characterized by the highest malignant cell percentage relative to other GC TME types. In some embodiments, GC TME type D is characterized by minimal or complete absence of leukocyte/lymphocyte infiltration (e.g., relative to other GC TME types). In some embodiments, GC TME type D is characterized by a high level of tumor proliferation rate relative to other GC TME types.

In some embodiments, GC TME type E is also referred to as “B-cell enriched” type. In some embodiments, GC TME type E is characterized by high levels of immune infiltrate with a significant number of B-cells, relative to other GC TME types. In some embodiments, GC TME type D is characterized by having a low proliferation rate relative to other GC TME types. In some embodiments, subjects having GC TME type E have been observed to have better prognosis (e.g., by overall survival (OS)) relative to other GC TME types.

Tables 2, 3, 4, 5, and 6 below describe examples of GC TME signatures and gene group scores produced by ssGSEA analysis and normalization (e.g., median scaling) of expression data from one or more GC subjects.

TABLE 2
Statistics of GC TME signatures of samples having GC TME type A. The statistics in
the table show, for each gene group score in the GC TME signature, the mean, minimum,
maximum, 25th percentile, 50th percentile, and 75% percentile values.

| Type A, | | | | | | |
|---|---|---|---|---|---|---|
| Mesenchymal, EMT | mean | min | max | 25% | 50% | 75% |
| CAF | 0.30 | −3.43 | 3.28 | −0.28 | 0.42 | 0.97 |
| Angiogenesis | 0.33 | −3.44 | 3.63 | −0.44 | 0.29 | 1.15 |
| Neutrophil signature | 0.26 | −2.46 | 4.00 | −0.44 | 0.19 | 0.92 |
| Protumor cytokines | 0.68 | −2.54 | 4.00 | −0.04 | 0.52 | 1.40 |
| MDSC | −0.20 | −4.00 | 2.69 | −0.83 | −0.24 | 0.40 |
| M2 signature | 0.18 | −3.02 | 3.81 | −0.46 | 0.19 | 0.85 |
| M1 signature | −0.68 | −3.98 | 3.21 | −1.29 | −0.69 | −0.03 |
| Antitumor cytokines | −0.66 | −4.00 | 3.44 | −1.28 | −0.68 | −0.02 |
| MHCII | −0.03 | −4.00 | 3.08 | −0.49 | 0.01 | 0.47 |
| Coactivation | −0.28 | −3.93 | 3.18 | −0.88 | −0.32 | 0.25 |
| molecules | | | | | | |
| Treg | −0.28 | −4.00 | 3.47 | −0.90 | −0.30 | 0.24 |
| Effector cells | −0.14 | −2.56 | 4.00 | −0.71 | −0.14 | 0.36 |
| T cells | 0.07 | −4.00 | 3.08 | −0.49 | 0.13 | 0.67 |
| NK cells | −0.17 | −3.29 | 3.36 | −0.64 | −0.17 | 0.30 |
| Checkpoint | −0.24 | −2.93 | 3.10 | −0.72 | −0.21 | 0.22 |
| inhibition | | | | | | |
| T cell traffic | −0.41 | −4.00 | 2.54 | −1.09 | −0.38 | 0.22 |
| MHCI | −0.71 | −4.00 | 2.72 | −1.35 | −0.63 | −0.05 |
| B cells | 0.41 | −2.29 | 3.82 | −0.41 | 0.32 | 1.18 |
| Lgr5 ISC | 1.24 | −1.85 | 4.00 | 0.59 | 1.23 | 1.93 |
| Proliferation rate | −1.59 | −4.00 | 0.93 | −2.21 | −1.44 | −0.82 |

TABLE 3
Statistics of GC TME signatures of samples having GC TME type B. The statistics in
the table show, for each gene group score in the GC TME signature, the mean, minimum,
maximum, 25th percentile, 50th percentile, and 75% percentile values.

| Type B, | | | | | | |
|---|---|---|---|---|---|---|
| Immune | | | | | | |
| enriched, non | | | | | | |
| fibrotic (IE) | mean | min | max | 25% | 50% | 75% |
| CAF | −0.56 | −4.00 | 2.83 | −1.32 | −0.51 | 0.26 |
| Angiogenesis | −0.47 | −4.00 | 3.94 | −1.20 | −0.48 | 0.26 |
| Neutrophil | 0.07 | −4.00 | 4.00 | −0.75 | −0.01 | 0.82 |
| signature | | | | | | |
| Protumor | −0.33 | −4.00 | 3.37 | −1.00 | −0.36 | 0.32 |
| cytokines | | | | | | |
| MDSC | 0.59 | −2.80 | 3.92 | −0.17 | 0.66 | 1.39 |
| M2 signature | 0.46 | −3.26 | 4.00 | −0.29 | 0.47 | 1.30 |
| M1 signature | 0.60 | −3.13 | 4.00 | −0.20 | 0.53 | 1.31 |
| Antitumor | 0.86 | −2.88 | 3.97 | 0.13 | 0.87 | 1.55 |
| cytokines | | | | | | |
| MHCII | 0.65 | −4.00 | 3.18 | 0.12 | 0.67 | 1.15 |
| Coactivation | 1.00 | −2.36 | 4.00 | 0.14 | 0.98 | 1.78 |
| molecules | | | | | | |
| Treg | 0.98 | −1.86 | 4.00 | 0.17 | 0.92 | 1.68 |
| Effector cells | 1.39 | −1.47 | 4.00 | 0.62 | 1.38 | 2.09 |
| T cells | 0.79 | −2.07 | 4.00 | 0.09 | 0.76 | 1.48 |
| NK cells | 1.52 | −1.91 | 4.00 | 0.69 | 1.52 | 2.26 |
| Checkpoint | 1.25 | −1.93 | 4.00 | 0.38 | 1.19 | 2.05 |
| inhibition | | | | | | |
| T_cell_traffic | 1.21 | −2.56 | 3.88 | 0.57 | 1.34 | 1.98 |
| MHCI | 1.19 | −3.52 | 4.00 | 0.50 | 1.30 | 1.92 |
| B_cells | 0.27 | −2.82 | 4.00 | −0.49 | 0.11 | 0.88 |
| Lgr5 ISC | −0.96 | −4.00 | 2.07 | −1.58 | −0.97 | −0.26 |
| Proliferation rate | 0.54 | −2.33 | 3.26 | 0.06 | 0.55 | 1.00 |

TABLE 4
Statistics of GC TME signatures of samples having GC TME type C. The statistics in
the table show, for each gene group score in the GC TME signature, the mean, minimum,
maximum, 25th percentile, 50th percentile, and 75% percentile values.
Type C, Fibrotic

| (F). | mean | min | max | 25% | 50% | 75% |
|---|---|---|---|---|---|---|
| CAF | 0.77 | −3.39 | 3.62 | 0.05 | 0.81 | 1.48 |
| Angiogenesis | 0.83 | −2.36 | 4.00 | 0.06 | 0.76 | 1.54 |
| Neutrophil | 0.56 | −3.51 | 4.00 | −0.39 | 0.30 | 1.38 |
| signature | | | | | | |
| Protumor cytokines | 0.78 | −2.78 | 4.00 | −0.08 | 0.73 | 1.61 |
| MDSC | 0.56 | −4.00 | 4.00 | −0.25 | 0.55 | 1.32 |
| M2 signature | 0.09 | −3.91 | 3.63 | −0.66 | 0.05 | 0.80 |
| M1 signature | 0.74 | −2.10 | 4.00 | −0.08 | 0.67 | 1.47 |
| Antitumor | 0.68 | −3.12 | 4.00 | −0.02 | 0.63 | 1.39 |
| cytokines | | | | | | |
| MHCII | −0.56 | −4.00 | 3.01 | −1.25 | −0.41 | 0.24 |
| Coactivation | 0.02 | −2.47 | 3.54 | −0.61 | −0.07 | 0.60 |
| molecules | | | | | | |
| Treg | −0.29 | −3.63 | 4.00 | −1.00 | −0.38 | 0.38 |
| Effector cells | −0.48 | −3.37 | 3.35 | −1.06 | −0.54 | 0.07 |
| T cells | −0.64 | −3.70 | 2.11 | −1.29 | −0.59 | −0.01 |
| NK cells | −0.40 | −2.97 | 3.74 | −0.97 | −0.42 | 0.13 |
| Checkpoint | −0.26 | −4.00 | 3.27 | −0.84 | −0.25 | 0.30 |
| inhibition | | | | | | |
| T cell traffic | 0.04 | −2.47 | 3.05 | −0.62 | 0.01 | 0.69 |
| MHCI | −0.15 | −4.00 | 2.39 | −0.78 | −0.14 | 0.50 |
| B cells | −0.47 | −4.00 | 2.81 | −1.06 | −0.52 | 0.04 |
| Lgr5 ISC | −0.14 | −3.57 | 3.65 | −0.85 | −0.08 | 0.51 |
| Proliferation rate | 0.03 | −3.37 | 3.59 | −0.54 | 0.10 | 0.62 |

TABLE 5
Statistics of GC TME signatures of samples having GC TME type D. The statistics in
the table show, for each gene group score in the GC TME signature, the mean, minimum,
maximum, 25th percentile, 50th percentile, and 75% percentile values.

| Type D, Immune | | | | | | |
|---|---|---|---|---|---|---|
| desert. | mean | min | max | 25% | 50% | 75% |
| CAF | −0.86 | −4.00 | 2.67 | −1.52 | −0.80 | −0.13 |
| Angiogenesis | −0.71 | −4.00 | 2.26 | −1.41 | −0.69 | 0.00 |
| Neutrophil | −0.55 | −4.00 | 4.00 | −1.24 | −0.58 | 0.08 |
| signature | | | | | | |
| Protumor cytokines | −0.72 | −4.00 | 3.21 | −1.38 | −0.82 | −0.15 |
| MDSC | −0.95 | −4.00 | 1.97 | −1.63 | −0.96 | −0.36 |
| M2 signature | −1.19 | −4.00 | 1.50 | −1.85 | −1.17 | −0.49 |
| M1 signature | −0.65 | −4.00 | 2.52 | −1.36 | −0.63 | 0.05 |
| Antitumor | −0.85 | −4.00 | 2.18 | −1.50 | −0.84 | −0.26 |
| cytokines | | | | | | |
| MHCII | −1.29 | −4.00 | 1.82 | −2.14 | −1.14 | −0.31 |
| Coactivation | −0.81 | −3.55 | 4.00 | −1.46 | −0.87 | −0.20 |
| molecules | | | | | | |
| Treg | −0.26 | −3.05 | 4.00 | −0.98 | −0.38 | 0.35 |
| Effector cells | −0.69 | −3.71 | 2.69 | −1.24 | −0.71 | −0.10 |
| T cells | −0.93 | −3.83 | 2.19 | −1.60 | −0.92 | −0.24 |
| NK cells | −0.67 | −3.89 | 2.50 | −1.22 | −0.66 | −0.13 |
| Checkpoint | −0.76 | −4.00 | 2.32 | −1.37 | −0.77 | −0.12 |
| inhibition | | | | | | |
| T cell traffic | −0.90 | −3.85 | 2.05 | −1.57 | −0.89 | −0.23 |
| MHCI | −0.48 | −4.00 | 2.43 | −1.22 | −0.40 | 0.43 |
| B cells | −0.35 | −4.00 | 3.40 | −0.96 | −0.41 | 0.21 |
| Lgr5 ISC | 0.16 | −2.56 | 4.00 | −0.74 | 0.04 | 0.86 |
| Proliferation rate | 0.72 | −2.39 | 3.50 | 0.18 | 0.73 | 1.24 |

TABLE 6
Statistics of GC TME signatures of samples having GC TME type E. The statistics in
the table show, for each gene group score in the GC TME signature, the mean, minimum,
maximum, 25th percentile, 50th percentile, and 75% percentile values.

| Type E, B-cell | | | | | | |
|---|---|---|---|---|---|---|
| enriched | mean | min | max | 25% | 50% | 75% |
| CAF | −0.10 | −3.90 | 3.13 | −0.66 | −0.06 | 0.53 |
| Angiogenesis | −0.14 | −3.09 | 3.86 | −0.84 | −0.09 | 0.63 |
| Neutrophil signature | 0.07 | −2.85 | 4.00 | −0.68 | 0.00 | 0.70 |
| Protumor cytokines | −0.18 | −3.05 | 3.92 | −0.90 | −0.30 | 0.51 |
| MDSC | 0.02 | −4.00 | 3.63 | −0.68 | 0.00 | 0.67 |
| M2 signature | 0.36 | −2.81 | 4.00 | −0.32 | 0.31 | 1.10 |
| M1 signature | 0.06 | −4.00 | 4.00 | −0.64 | 0.02 | 0.66 |
| Antitumor cytokines | 0.02 | −2.66 | 4.00 | −0.56 | −0.05 | 0.62 |
| MHCII | 0.60 | −1.82 | 2.60 | 0.17 | 0.62 | 1.02 |
| Coactivation molecules | 0.96 | −2.23 | 4.00 | 0.17 | 0.92 | 1.70 |
| Treg | 0.63 | −2.97 | 4.00 | −0.12 | 0.57 | 1.48 |
| Effector cells | 0.96 | −1.51 | 4.00 | 0.21 | 0.86 | 1.61 |
| T cells | 1.30 | −1.16 | 4.00 | 0.67 | 1.26 | 1.88 |
| NK cells | 0.81 | −2.32 | 4.00 | 0.16 | 0.73 | 1.39 |
| Checkpoint inhibition | 0.94 | −1.99 | 4.00 | 0.16 | 0.80 | 1.56 |
| T cell traffic | 0.46 | −2.88 | 2.97 | −0.20 | 0.45 | 1.18 |
| MHCI | 0.30 | −3.93 | 2.92 | −0.29 | 0.23 | 0.96 |
| B cells | 1.62 | −2.00 | 4.00 | 0.88 | 1.50 | 2.31 |
| Lgr5 ISC | 0.10 | −3.01 | 2.23 | −0.41 | 0.00 | 0.65 |
| Proliferation rate | −0.88 | −3.94 | 2.62 | −1.43 | −0.83 | −0.27 |

In some embodiments, the present disclosure provides methods for identifying a subject having, suspected of having, or at risk of having GC as having an increased likelihood of having a good prognosis (e.g., as measured by overall survival (OS) or progression-free survival (PFS).

In some embodiments, the method comprises determining a GC TME type of the subject as described herein.

In some embodiments, the methods comprise identifying the subject as having a decreased risk of GC progression relative to other GC TME types when the subject is assigned GC TME type E. In some embodiments, “decreased risk of GC progression” may indicate better prognosis of GC or decreased likelihood of having advanced disease in a subject. In some embodiments, “decreased risk of GC progression” may indicate that the subject who has GC is expected to be more responsive to certain treatments. For instance, “decreased risk of GC progression” indicates that a subject is at least 10%, 20%, 30%, 40%, 50%, 60%, 70%, 80%, 90%, or 100% likely to experience a progression-free survival event (e.g., relapse, retreatment, or death) than another GC patient or population of GC patients (e.g., patients having GC, but not the same GC TME type as the subject).

In some embodiments, the methods further comprise identifying the subject as having an increased risk of GC progression relative to other GC TME types when the subject is assigned a GC TME type other than GC TME type E. In some embodiments, “increased risk of GC progression” may indicate less positive prognosis of GC or increased likelihood of having advanced disease in a subject. In some embodiments, “increased risk of GC progression” may indicate that the subject who has GC is expected to be less responsive or unresponsive to certain treatments and show less or no improvements of disease symptoms. For instance, “increased risk of GC progression” indicates that a subject is at least 10%, 20%, 30%, 40%, 50%, 60%, 70%, 80%, 90%, or 100% more likely to experience a progression-free survival event (e.g., relapse, retreatment, or death) than another GC patient or population of GC patients (e.g., patients having GC, but not the same GC TME type as the subject).

The disclosure is based, in part, on the recognition that subjects having certain GC TME types (e.g., GC TME type E) are characterized as having (or being more likely to have) tertiary lymphoid structures (TLS), compared to subjects having other types of GC TME. Tertiary lymphoid structures are ectopic lymphoid organs that develop in non-lymphoid tissues at sites of chronic inflammation including tumors, for example as described by Sautès-Fridman et al. Nat Rev Cancer 19, 307-325 (2019). In the context of certain cancers (e.g., gastric cancers) the presence of TLS is associated with an improved subject prognosis. Accordingly, in some embodiments, subjects identified as having GC TME type E have an increased likelihood of having PFS or increased overall survival (OS) relative to subjects having other GC TME types.

In some embodiments, the methods described herein comprise the use of at least one computer hardware processor to perform the determination.

In some embodiments, the present disclosure provides a method for providing a prognosis, predicting survival, or stratifying patient risk of a subject suspected of having, or at risk of having GC. In some embodiments, the method comprises determining a GC TME type of the subject as described herein.

### Updating GC TME Clusters Based on New Data

Techniques for generating GC TME clusters are described herein. It should be appreciated that the GC TME clusters may be updated as additional GC TME signatures are computed for patients. In some embodiments, the GC TME signature of the subject is one of a threshold number GC TME signatures for a threshold number of subjects. In some embodiments, when the threshold number of GC TME signatures is generated the GC TME signature clusters are updated. For example, once a threshold number of new GC TME signatures are obtained (e.g., 1 new signature, 10 new signatures, 100 new signatures, 500 new signatures, any suitable threshold number of signatures in the range of 10-1,000 signatures), the new signatures may be combined with the GC TME signatures previously used to generate the GC TME clusters and the combined set of old and new GC TME signatures may be clustered again (e.g., using any of the clustering algorithms described herein or any other suitable clustering algorithm) to obtain an updated set of GC TME signature clusters.

In this way, data obtained from a future patient may be analyzed in a way that takes advantage of information learned from patients whose GC TME signature was computed prior to that of the future patient. In this sense, the machine learning techniques described herein (e.g., the unsupervised clustering machine learning techniques) are adaptive and learn with the accumulation of new patient data. This facilitates improved characterization of the GC TME type that future patients may have and may improve the selection of treatment for those patients.

### Anti-Cancer Therapies

Aspects of the disclosure relate to methods of treating a subject having (or suspected or at risk of having) GC based upon a determination of the GC TME type of the subject. In some embodiments, the methods comprise administering one or more (e.g., 1, 2, 3, 4, 5, or more) therapeutic agents to the subject. In some embodiments, the therapeutic agent (or agents) administered to the subject are selected from small molecules, peptides, nucleic acids, radioisotopes, cells (e.g., CAR T-cells, etc.), and combinations thereof. Examples of therapeutic agents include chemotherapies (e.g., cytotoxic agents, etc.), immunotherapies (e.g., immune checkpoint inhibitors, such as PD-1 inhibitors, PD-L1 inhibitors, etc.), antibodies (e.g., anti-HER2 antibodies), cellular therapies (e.g. CAR T-cell therapies), gene silencing therapies (e.g., interfering RNAs, CRISPR, etc.), antibody-drug conjugates (ADCs), and combinations thereof.

In some embodiments, a subject is administered an effective amount of a therapeutic agent. “An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor microenvironment, tumor formation, tumor growth, or GC TME types, etc.) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays and/or by monitoring GC TME types as described herein. The dosing regimen (including the therapeutic used) may vary over time.

When the anti-cancer therapeutic agent is not an antibody, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known in the art).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of GC, or the predisposition toward GC.

Alleviating GC includes delaying the development or progression of the disease, or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. Alternatively, or in addition to the clinical techniques known in the art, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor, a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, R-CHOP, Carboplatin or Cisplatin, Docetaxel, Gemcitabine, Nab-Paclitaxel, Paclitaxel, Pemetrexed, and Vinorelbine. Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

In some aspects, the disclosure provides a method for treating gastric cancer, the method comprising administering one or more therapeutic agents (e.g., one or more anti-cancer agents, such as one or more chemotherapeutic agents) to a subject identified as having a particular GC TME type, wherein the GC TME type of the subject has been identified by method as described by the disclosure.

In some embodiments, a subject identified as having GC TME type E is administered an immunotherapy (e.g., an immune checkpoint inhibitor, such as pembrolizumab). Dosages of pembrolizumab are well known, for example 200 mg every 3 weeks or 400 mg every 6 weeks, by infusion over 30 minutes.

### Reports

In some aspects, methods disclosed herein comprise generating a report for assisting with the preparation of recommendation for prognosis and/or treatment. The generated report can provide summary of information, so that the clinician can identify the GC TME type or suitable therapy. The report as described herein may be a paper report, an electronic record, or a report in any format that is deemed suitable in the art. The report may be shown and/or stored on a computing device known in the art (e.g., handheld device, desktop computer, smart device, website, etc.). The report may be shown and/or stored on any device that is suitable as understood by a skilled person in the art.

In some embodiments, methods disclosed herein can be used for commercial diagnostic purposes. For example, the generated report may include, but is limited to, information concerning expression levels of one or more genes from any of the gene groups described herein, clinical, and pathologic factors, patient's prognostic analysis, predicted response to the treatment, classification of the GC TME environment (e.g., as belonging to one of the types described herein), the alternative treatment recommendation, and/or other information. In some embodiments, the methods and reports may include database management for the keeping of the generated reports. For instance, the methods as disclosed herein can create a record in a database for the subject (e.g., subject 1, subject 2, etc.) and populate the specific record with data for the subject. In some embodiments, the generated report can be provided to the subject and/or to the clinicians. In some embodiments, a network connection can be established to a server computer that includes the data and report for receiving or outputting. In some embodiments, the receiving and outputting of the date or report can be requested from the server computer.

### Computer Implementation

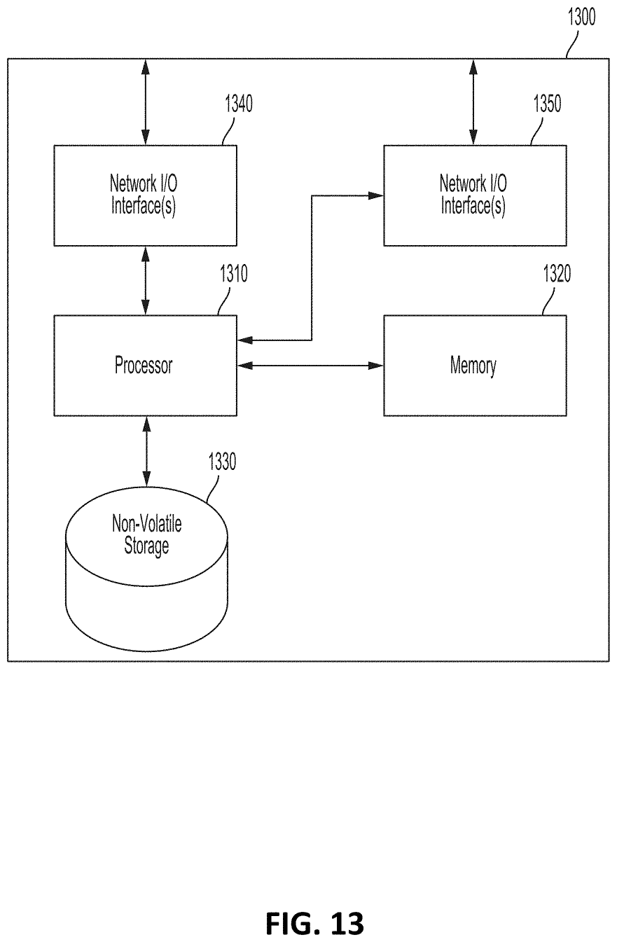

An illustrative implementation of a computer system 1300 that may be used in connection with any of the embodiments of the technology described herein (e.g., such as the method of FIG. 1) is shown in FIG. 13. The computer system 1300 includes one or more processors 1310 and one or more articles of manufacture that comprise non-transitory computer-readable storage media (e.g., memory 1320 and one or more non-volatile storage media 1330).

The processor 1310 may control writing data to and reading data from the memory 1320 and the non-volatile storage device 1330 in any suitable manner, as the aspects of the technology described herein are not limited to any particular techniques for writing or reading data. To perform any of the functionality described herein, the processor 1310 may execute one or more processor-executable instructions stored in one or more non-transitory computer-readable storage media (e.g., the memory 1320), which may serve as non-transitory computer-readable storage media storing processor-executable instructions for execution by the processor 1310.

Computing device 1300 may also include a network input/output (I/O) interface 1340 via which the computing device may communicate with other computing devices (e.g., over a network), and may also include one or more user I/O interfaces 1350, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions.

The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

### EXAMPLES

### Example 1: Identification of Gastric Cancer Tumor Microenvironment (TME)

This example describes an illustrative technique for generating an GC TME signature for a subject from RNA expression data for the subject, according to some embodiments of the technology described herein. The produced GC TME signature reflects and/or indicates the abundance of both the malignant and microenvironment (TME) cell subpopulations and the activity of tumor-promoting and tumor-suppressive processes occurring within a tumor, and constitutes a personalized tumor map.

The generated GC TME signature for the subject is used to identify an GC TME type for the subject from among five GC TME types: GC TME type A, GC TME type B, GC TME type C, GC TME type D, and GC TME type E.

Aspects of some of the steps of the process described in this example are described in further detail herein including with reference to FIGS. 1-4 above.

RNA expression data (including both RNA-seq and microarray expression data) were obtained from multiple public databases. Data were subjected to basic quality control (QC) measures. For example, outlier samples and samples with signs of RNA degradation were excluded. Preprocessing of expression data included normalization and log-transformation. For microarrays normalization is performed automatically using gcrma package. RNA-seq data was subsequently normalized to TPM (transcript per million) units. TPM normalization techniques are described in Wagner et al. (Theory Biosci. (2012) 131:281-285), which is incorporated by reference herein in its entirety. TPM normalization may be performed using a software package, such as, for example, the gcrma package. Aspects of the gcrma package are described in Wu J, Gentry RIwcfJMJ (2021). “gcrma: Background Adjustment Using Sequence Information. R package version 2.66.0.”, which is incorporated by reference in its entirety herein. In some embodiments, RNA expression level in TPM units for a particular gene may be calculated according to:

$$A \cdot \frac{1}{\sum ( A )} \cdot {10}^{6} \\ Where A = \frac{total reads mapped to gene \cdot {10}^{3}}{gene length in bp}$$

The GC TME type determined for a subject is determined using a GC TME signature. The GC TME signature includes gene group scores (e.g., one or more of the gene groups described in Table 1) obtained using ssGSEA. The gene group scores in the GC TME signatures were calculated from log-transformed RNA expression valued. The ssGSEA was performed according to the following formula:

$$ssGSEA score = \frac{{\sum }_{i}^{N} {r}_{i}^{1.25}}{{{\sum }^{N}}_{i} {r}_{i}^{0.25}} - \frac{( M - N + 1 )}{2}$$

where rᵢ represents the rank of the ith gene in expression matrix, where N represents the number of genes in the gene set (e.g., the number of genes in the first gene group when ssGSEA is being used to determine a score for the first gene group using expression levels of the genes in the first gene group), and where M represents total number of genes in expression matrix. Additional, suitable techniques of performing GSEA are known in the art and are contemplated for use in the methods described herein without limitation. In some embodiments, a GC signature is calculated by performing ssGSEA on expression data from a plurality of subjects, for example expression data from one or more cohorts of subjects.

After calculation, the scores were scaled using rank estimation and median-scaling, which was important for removing undesirable batch effects and to enable all the datasets to be combined together. Median scaling consisted of estimating median and MAD (median absolute deviation) for each signature within each dataset, and applying the formula xi-median(x)/MAD(x).

After GC TME signatures were calculated according to the above for multiple patients, unsupervised clustering was performed to generate GC TME clusters. To classify a new sample, it is grouped together with the dataset used to get the GC TME type. Scores are calculated for the sample and scaled together with the selected cohort. After that the sample type, can be predicted by applying a machine learning model (e.g., K-nearest neighbor, “knn”) trained on the scaled metacohort.

In this example, inter-sample similarity was calculated using Pearson correlation. The distance matrix was converted into a graph where each sample formed a node and two nodes formed an edge with a weight equal to their Pearson correlation coefficient. Edges with weight lower than a specified threshold were removed. The Louvain community detection algorithm was applied to calculate graph partitioning into clusters. To mathematically determine the optimum weight threshold for observed clusters, minimum DaviesBouldin, maximum Calinski-Harabasz, and Silhouette techniques were employed. Separations with low-populated clusters (<5% of samples) were excluded. This analysis resulted in the generation of five (5) GC TME signature clusters, corresponding to five (5) GC TME types.

### Example of Characterizing GC TME Types

Using the aforementioned approach on several publicly available cancer data sets, five distinct types of GC were observed (FIG. 5A).

To identify GC TME types, a meta-cohort of gene expression data was collected from public datasets. GC TME signatures comprising twenty gene group scores were used to estimate different biological processes in each of the samples. Genes included in each gene group are described in Table 1. To overcome batch effect from different datasets, rank estimation and median scaling transformation were used prior to clustering, which led to identification of the five GC TME types. Examples of processes for identifying GC TME types are described in FIG. 1. Using unsupervised clustering, five stable GC TME types were identified (FIG. 5A): Immune Enriched/Non fibrotic (type B), Depleted (type D), Mesenchymal/EMT (type A), Fibrotic (type C), and “B cell-enriched” (type E).

FIG. 5B provides a heatmap of pairwise signature correlation for the gene groups listed in Table 1. Data indicate the gene group scores for the NK cells group, T cells group, B cells group, Treg group, MDSC group, CAF group, Proliferation rate group, and Lgr5 ISC group may facilitate typing the GC TME of a subject. Accordingly, in some embodiments, a GC TME signature may include gene group scores for these gene groups. Though, in other embodiments, one or more gene scores for one or more other gene groups may be used in addition to or instead of these gene group scores.

GC TME type E is characterized by the following features: low or medium stromal/vascularized component (e.g., lower than GC TME type A and GC TME type C), medium or high immune component (e.g., similar to GC type B), highest B cell signal (e.g., of all GC TME types), and low or medium neoplasm properties (e.g., low expression level of Proliferation rate and Oncogenes signatures, higher level of Tumor suppressors signature, relative to other GC TME types).

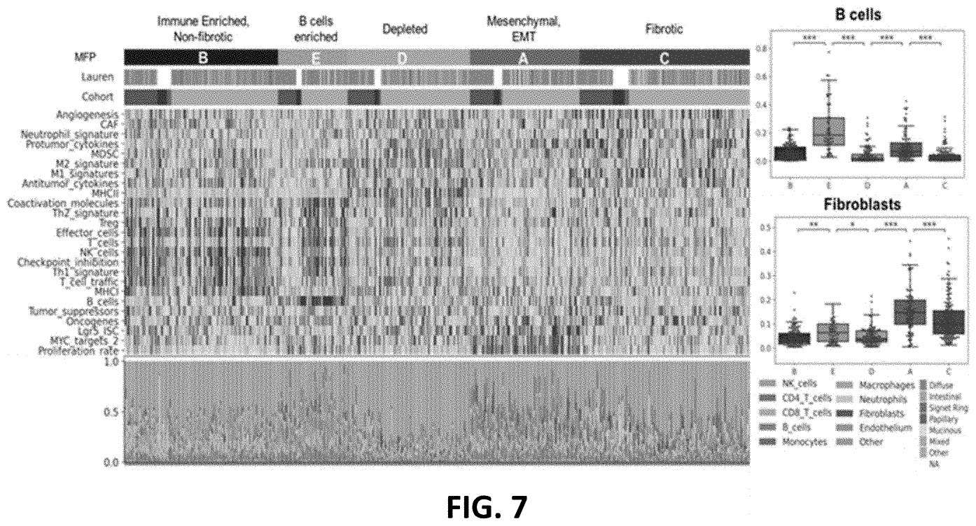

In some embodiments, GC TME type is prognostic for patient outcome. An analysis between GC TME type was performed and compared to TCGA histological data. FIG. 6 provides selected process signature comparisons across GC TME type. Data indicates that while GC TME type A and GC TME type C have the highest Angiogenesis and stromal (CAF=Cancer-associated fibroblasts) signals, GC TME type E represents the highest B cells signal and similar T cell signals. FIG. 7 provides an exemplary heatmap of GC TME type gene group signatures and cell deconvolution across the samples.

For RNA-seq samples, high B cell content was also supported by a cell deconvolution algorithm-based analysis. This algorithm allows reconstructing cell composition from bulk RNA-seq data and estimating the percentage of different cell types (fibroblasts, B cells, T cells, macrophages, etc.). GC TME type E samples proved the highest B cells percentage (FIG. 5A). Medians and cumulative distribution function (CDF) for each signature in each GC TME type are provided below in Tables 7 and 8.

TABLE 7
Median signature values by TME types in Gastric Cancer

| | A | B | C | D | E |
|---|---|---|---|---|---|
| Angiogenesis | 0.252944 | −0.41224 | 0.611601 | −0.56226 | −0.07151 |
| CAF | 0.348466 | −0.41863 | 0.671596 | −0.67448 | −0.05282 |
| Neutrophil signature | 0.171127 | −0.01064 | 0.282474 | −0.51952 | −0.00045 |
| Protumor cytokines | 0.436955 | −0.29628 | 0.589719 | −0.67449 | −0.23967 |
| MDSC | −0.19092 | 0.50296 | 0.437124 | −0.75104 | 0 |
| M2 signature | 0.159276 | 0.391135 | 0.039904 | −0.96645 | 0.270758 |
| M1 signature | −0.58993 | 0.423022 | 0.534411 | −0.54993 | 0.013511 |
| Coactivation molecules | −0.25588 | 0.8191 | −0.0597 | −0.70434 | 0.736773 |
| Antitumor cytokines | −0.55553 | 0.692828 | 0.508098 | −0.68359 | −0.04134 |
| MHCII | 0.00543 | 0.653321 | −0.38197 | −1.1027 | 0.610592 |
| Treg | −0.2602 | 0.773806 | −0.32024 | −0.33894 | 0.507427 |
| Effector cells | −0.11252 | 1.12815 | −0.44909 | −0.59551 | 0.699259 |
| T cells | 0.108771 | 0.614987 | −0.47069 | −0.71112 | 0.995619 |
| NK cells | −0.14148 | 1.263625 | −0.37989 | −0.6012 | 0.611168 |
| Checkpoint inhibition | −0.19267 | 1.04053 | −0.22015 | −0.67449 | 0.701052 |
| T cell traffic | −0.29942 | 1.014413 | 0.009406 | −0.69793 | 0.347058 |
| MHCI | −0.52738 | 1.068323 | −0.11561 | −0.32664 | 0.193498 |
| B cells | 0.257403 | 0.089833 | −0.46515 | −0.35592 | 1.238358 |
| Lgr5 ISC | 0.997763 | −0.77192 | −0.06831 | 0.039205 | 0 |
| Proliferation rate | −1.18066 | 0.474955 | 0.079253 | 0.632717 | −0.7031 |

TABLE 8
Mean cumulative distribution function (CDF) of signatures
values by TME types in Gastric Cancer

| | A | B | C | D | E |
|---|---|---|---|---|---|
| Angiogenesis | 0.574 | 0.388 | 0.683 | 0.333 | 0.468 |
| CAF | 0.588 | 0.380 | 0.690 | 0.310 | 0.480 |
| Neutrophil signature | 0.545 | 0.495 | 0.592 | 0.349 | 0.493 |
| Protumor cytokines | 0.643 | 0.407 | 0.656 | 0.303 | 0.436 |
| MDSC | 0.443 | 0.630 | 0.624 | 0.268 | 0.499 |
| M2 signature | 0.552 | 0.613 | 0.524 | 0.237 | 0.590 |
| M1 signature | 0.328 | 0.628 | 0.657 | 0.336 | 0.507 |
| Coactivation molecules | 0.408 | 0.695 | 0.480 | 0.274 | 0.698 |
| Antitumor cytokines | 0.327 | 0.693 | 0.651 | 0.287 | 0.494 |
| MHCII | 0.512 | 0.709 | 0.394 | 0.262 | 0.702 |
| Treg | 0.409 | 0.702 | 0.406 | 0.409 | 0.627 |
| Effector cells | 0.446 | 0.775 | 0.355 | 0.304 | 0.708 |
| T cells | 0.523 | 0.682 | 0.340 | 0.276 | 0.799 |
| NK cells | 0.438 | 0.791 | 0.370 | 0.299 | 0.677 |
| Checkpoint inhibition | 0.421 | 0.742 | 0.415 | 0.295 | 0.696 |
| T cell traffic | 0.387 | 0.757 | 0.498 | 0.278 | 0.600 |
| MHCI | 0.324 | 0.768 | 0.458 | 0.400 | 0.571 |
| B cells | 0.565 | 0.531 | 0.346 | 0.376 | 0.807 |
| Lgr5 ISC | 0.771 | 0.265 | 0.456 | 0.517 | 0.515 |
| Proliferation rate | 0.192 | 0.673 | 0.537 | 0.722 | 0.303 |

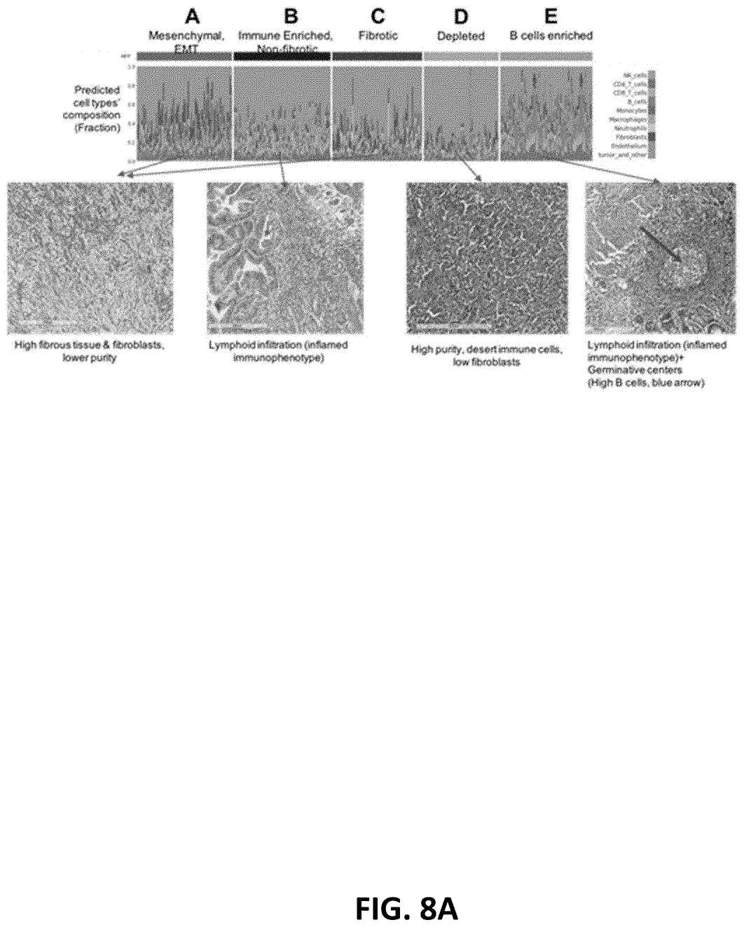

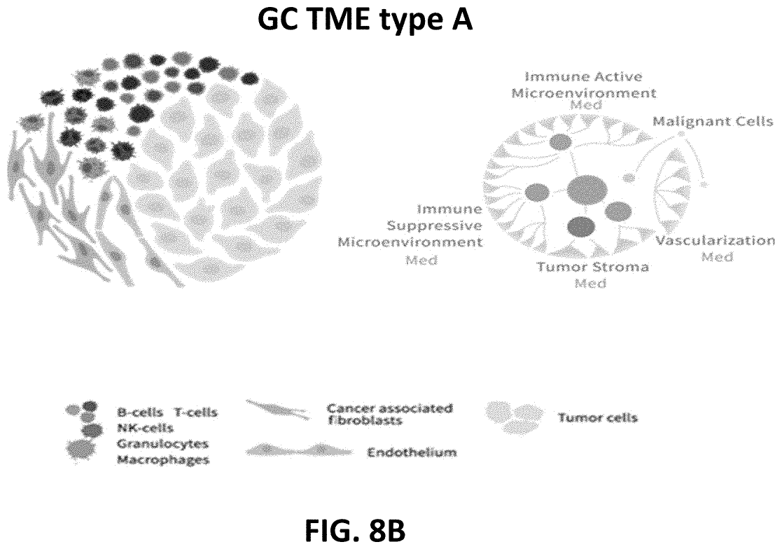

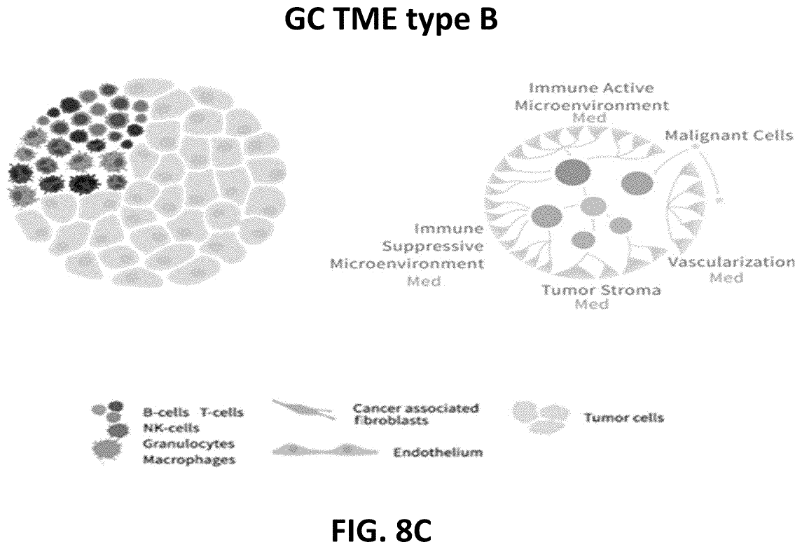

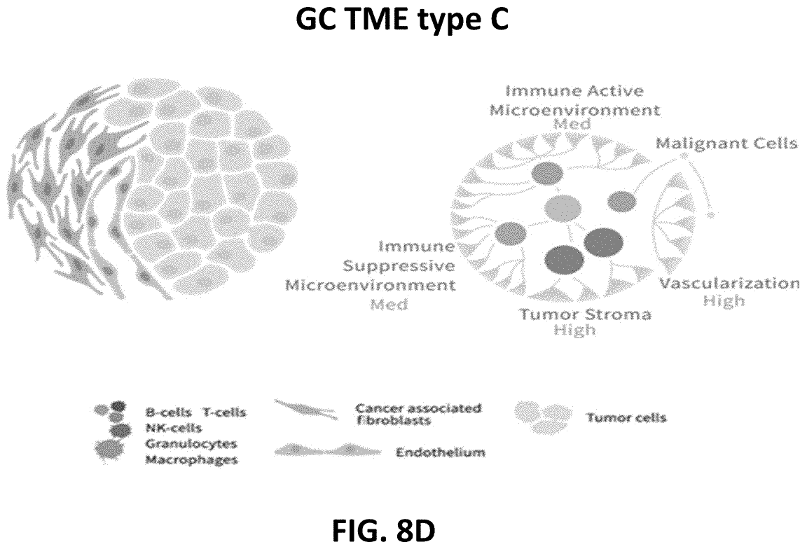

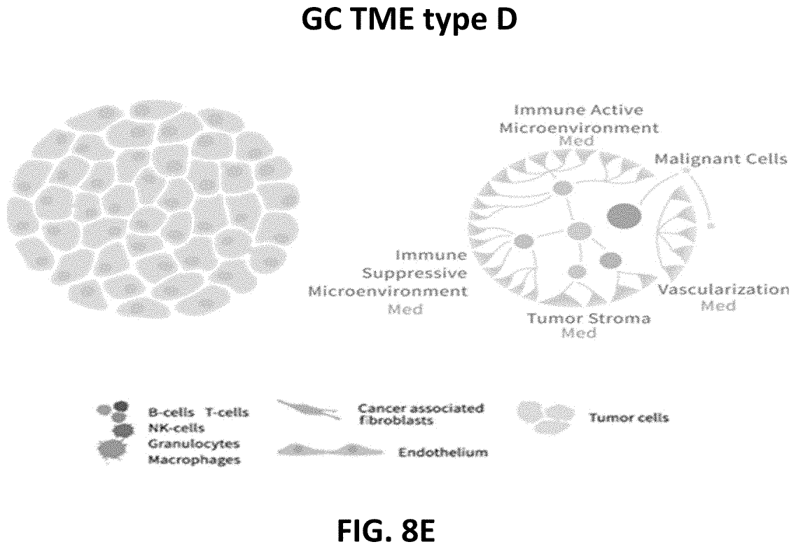

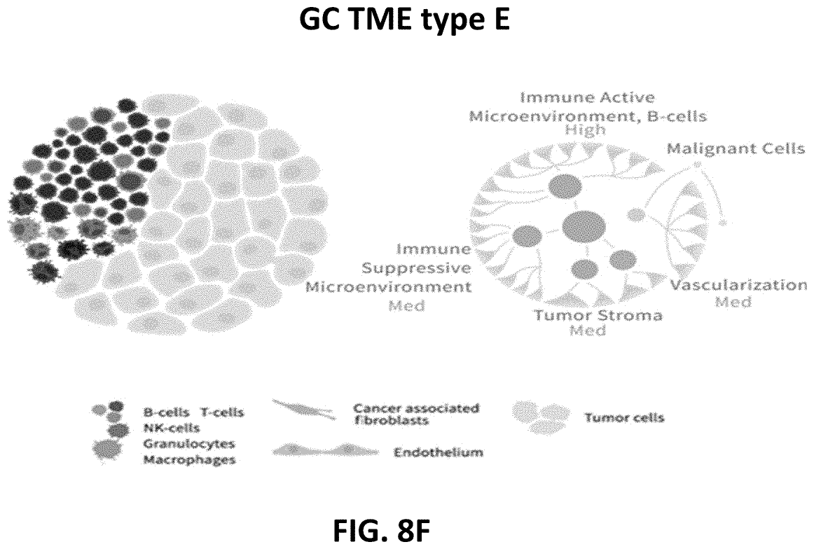

Using TCGA histology data, immune infiltration and stromal compartments were compared in samples belonging to different GC TME types (FIG. 8A). Samples of GC TME type A and GC TME type C had high stromal content. GC TME type D samples had desert immune, low fibroblast composition, and high tumor cellularity. GC TME type B and GC TME type E samples, on the contrary, showed high immune infiltration. Remarkably, GC TME type E samples also showed the existence of germinative centers/tertiary lymphoid structures (TLSs)—ectopic lymphoid organs that develop in non-lymphoid tissues (shown by the arrow in FIG. 8A).

FIGS. 8B-8F show graphic representations of relative cell type content of different GC TME types.

FIG. 8B shows a representation of the relative cell type content of GC TME type A, which is characterized by WNT-activation, and the prevalence of LGR5+ stem cells; type A was also associated with a low tumor proliferation rate. Cancer-associated fibroblasts are often observed. Signs of epithelial-mesenchymal transition (EMT) were present. Subjects having GC TME type A have been observed to have a poor prognosis.

FIG. 8C shows a representation of the relative cell type content of GC TME type B, which is characterized by high levels of tumor-infiltrating immune cells, including cytotoxic effector cells. This type has the highest tumor mutational burden (TMB). High PD-L1 expression is commonly observed. This GC TME type is often responsive to immunotherapy and associated with good prognosis.

FIG. 8D shows a representation of the relative cell type content of GC TME type C, which is associated with high fibrosis and dense collagen formation. This type is also characterized by minimal lymphocyte infiltration (non-inflamed) with intense angiogenesis. Cancer-associated fibroblasts (CAF) are abundant. High levels of both protumor and antitumor cytokines have been observed. This GC TME type is associated with poor prognosis. FIG. 8E shows a representation of the relative cell type content of GC TME type D, which is characterized by the highest percentage of malignant cells, while leukocyte/lymphocyte infiltration is only minimal or completely absent. A high tumor proliferation rate has been observed. This GC TME type is associated with good prognosis.

FIG. 8F shows a representation of the relative cell type content of GC TME type E, which is characterized by the presence of tertiary lymphoid structures (TLS), and high levels of immune infiltrate with a significant number of B cells. A low tumor proliferation rate has been observed. This GC TME type is associated with good prognosis.

Certain GC TME types were found to be associated with malignant properties: Mesenchymal A type comprised the majority of the studied metastatic samples and was characterized by the highest signal of LGR5+ stem cell signature.

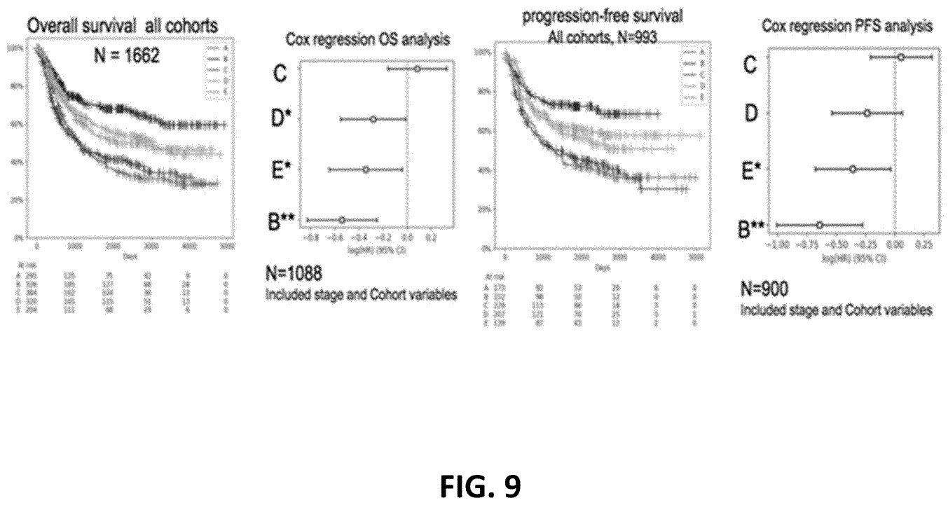

TLSs have been observed in various cancers: colorectal cancer, lung cancer, clear-cell renal cell carcinoma, sarcoma, urothelial bladder cancer, and others. The existence of TLS may provide additional opportunities in treatment decision making. For example, it has been observed that TLS-positive samples in primary gastrointestinal stromal tumors demonstrated better postoperative outcomes. Analysis of overall survival (OS) and progression-free survival (PFS) revealed better prognosis for GC TME type E compared with stromal enriched and WNT activated GC TME type A and GC TME type C (FIG. 9). Samples of GC TME types B, E and D had the best overall survival rates.

### Example 2: Additional Embodiments

Association of STAD TME Types with EBV, MSI, TMB Status

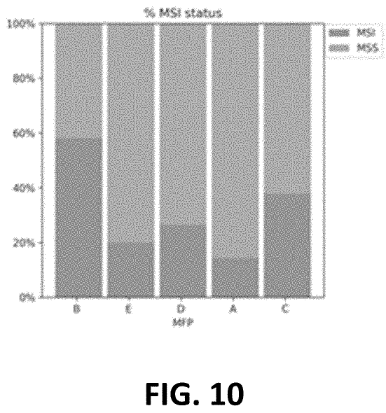

GC is histologically classified into intestinal, diffuse and the mixed types, and into four molecular types based on genetic profiling (i.e., microsatellite instable (MSI), EBV positive, chromosomal instable, and genomically stable). Here, samples with various statuses of EBV, MSI, and tumor mutational burden (TMB) across the GC TME types were examined. FIG. 10 shows comparison of MSS and MSI samples in different GC TME types.

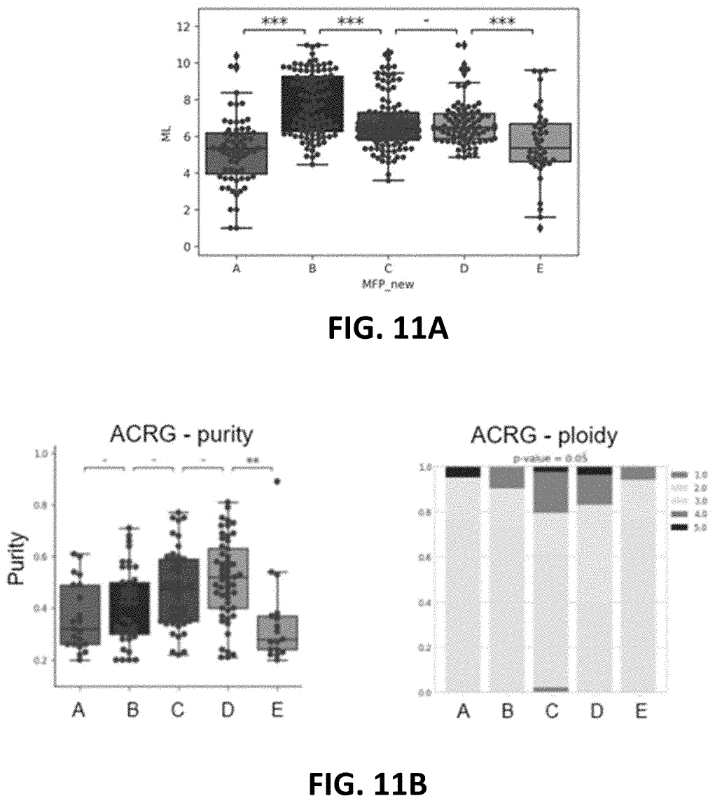

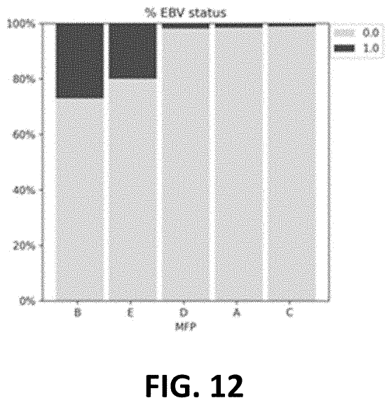

FIGS. 11A-11B show mutation burden across the GC TME type in the TCGA cohort. As shown in FIG. 11A, immune enriched type has the highest mutation load (ML) in comparison to all other GC TME types. However, in the ACGR cohort, the highest malignant cell content (i.e., purity or cellularity) and ploidy were observed in the immune depleted type, GC TME type D (FIG. 11B, left). Additional studies were conducted to examine the EBV status of the TCGA cohorts across all GC TME types. FIG. 12 shows strong relationships between EBV-positive status (i.e., “1.0”) and GC TME type B and GC TME type E (immune enriched and B cell enriched, respectively). The overall results confirmed that subjects who have GC TME type B and GC TME type E would be determined to have increased likelihood of having a good prognosis and/or progression-free survival (PFS).

TABLE 9
Exemplary NCBI Accession Numbers for genes listed in Table 1.

| Gene | Accession No. |
|---|---|
| ACTA2 | NM_001141945; NM_001320855; NM_001613 |
| ANGPT1 | NM_001314051; NM_001146; NM_001199859; NM_139290; XR_928319 |
| ANGPT2 | NM_001118888; NM_001386335; NM_001386337; NM_001118887; NM_001147; |
| | NM_001386336 |
| FASLG | NM_001302746; NM_000639 |
| ARG1 | NM_001369020; NM_000045; NM_001244438; NR_160934 |
| B2M | XR_002957658; NM_004048; XM_005254549 |
| CCND1 | NM_053056 |
| BCL2 | NM_000657; XM_011526135; XM_017025917; NM_000633; XR_935248 |
| TNFRSF17 | NM_001192 |
| BLK | NM_001330465; XM_011543829; XM_011543824; XM_011543827; XM_011543828; |
| | NM_001715; XM_011543825 |
| BUB1 | NM_004336; NM_001278617; XR_923001; NM_001278616 |
| CA9 | XR_428428; NM_001216; XR_001746374 |
| CCNB1 | NM_001354844; NM_031966; NM_001354845 |
| CCNE1 | XM_011527440; NM_001238; NM_001322259; NM_001322261; NM_001322262; |
| | NM_057182 |
| CD3D | NM_001040651; NM_000732 |
| CD3E | NM_000733 |
| CD3G | XM_005271724; XM_006718941; NM_000073 |
| CD5 | NM_014207; NM_001346456 |
| CD8A | NM_001145873; NM_001382698; NM_001768; NR_168478; NR_168479; |
| | NM_171827; NR_168480; NR_168481; NR_027353 |
| CD8B | NM_172102; NM_172100; NM_001178100; NM_004931; NM_172101; NM_172213; |
| | NM_172099; XM_011533164 |
| CD19 | NM_001178098; NM_001385732; NM_001770; XR_950871; XM_006721103; |
| | NR_169755; XM_011545981 |
| MS4A1 | NM_021950; NM_152866; NM_152867 |
| CD22 | NM_001185100; NM_001185099; NM_024916; NM_001185101; NM_001771; |
| | NM_001278417 |
| CD27 | XM_017020232; XM_017020233; NM_001242; XM_017020234; XM_011521042 |
| CD28 | XM_011512195; NM_006139; XM_011512197; NM_001243078; NM_001243077; |
| | XM_011512194 |
| CD80 | NM_005191 |
| CD86 | NM_001206924; NM_006889; NM_176892; NM_001206925; NM_175862 |
| CD40 | NM_001302753; NM_001322422; NM_152854; NM_001322421; NM_001362758; |
| | NM_001250; NR_136327; XM_011529109; XM_005260619; XM_017028135; |
| | XM_017028136; NR_126502 |
| CD40EG | NM_000074 |
| CD68 | NM_001251; NM_001040059 |
| CD70 | NM_001252; NM_001330332 |
| CD79A | NM_021601; NM_001783 |
| CD79B | NM_001039933; NM_021602; NM_000626; NM_001329050 |
| CDH5 | NM_001114117; XM_024450133; NM_001795; XM_011522801 |
| CDK2 | NM_001290230; XM_011537732; NM_052827; NM_001798 |
| CETN3 | NM_004365; NM_001297765; NM_001297768 |
| CCR8 | NM_005201 |
| CMKLR1 | NM_001142343; NM_001142345; XM_017018820; NM_001142344; NM_004072 |
| C0L1A1 | XM_005257058; XM_005257059; XM_011524341; NM_000088 |
| COL1A2 | NM_000089 |
| COL3A1 | NM_000090; NM_001376916 |
| COL4A1 | NM_001845; XM_011521048; NM_001303110 |
| COL5A1 | XM_017014266; XR_001746183; NM_000093; NM_001278074 |
| COL6A1 | NM_001848 |
| COL6A2 | NM_001849; NM_058175; NM_058174 |
| COL6A3 | NM_057164; NM_057167; NM_057166; NM_004369; NM_057165 |
| COL11A1 | XM_017000337; XM_017000335; XM_017000336; NR_134980; NM_080629; |
| | XM_017000334; NM_001190709; NM_001854; NM_080630 |
| CR2 | NM_001877; NM_001006658; XM_011509206; |
| CSF1R | NM_001375320; NM_005211; NR_164679; NM_001349736; NM_001288705; |
| | NM_001375321; NR_109969 |
| CTLA4 | NM_005214; NM_001037631 |
| CTSG | NM_001911; XM_011536499 |
| CX3CR1 | NM_001171174; NM_001337; NM_001171171; NM_001171172 |
| CYBB | NM_000397 |
| CYP2E1 | NM_000773 |
| DGKG | NM_001346; NM_001080745; NM_001080744 |
| E2F1 | NM_005225 |
| ELANE | XM_011527776; XM_011527775; NM_001972 |
| EPHA4 | NM_001304537; NM_001363748; NM_004438; NM_001304536 |
| FAP | XM_011510797; NM_004460; XM_011510796; XR_001738668; XM_017003585; |
| | XR_922891; NM_001291807 |
| FBLN1 | NM_006485; NM_006486; NM_001996; NM_006487 |
| FCGR3B | ; NM_001271036; NM_001271037; NM_001244753; NM_000570; NM_001271035 |
| FGFR4 | NM_213647; NM_022963; NM_002011; NM_001291980; NM_001354984 |
| FLT1 | NM_001160030; NM_001159920; XM_011535014; XM_017020485; NM_001160031; |
| | NM_002019 |
| FN1 | NM_001306129; NM_001365519; NM_212474; NM_001365517; NM_001306132; |
| | NM_001365522; NM_001306131; NM_001365521; NM_212476; NM_212478; |
| | NM_212475; NM_001365523; NM_001365524; NM_002026; NM_001365520; |
| | NM_212482; NM_001365518; NM_054034; NM_001306130 |
| FFAR2 | NM_005306; NM_001370087; XM_017026711 |
| GRK4 | XM_011513452; XM_017008058; XM_005247962; XM_011513447; XM_011513449; |
| | XM_011513455; XM_017008052; XM_017008059; XM_017008064; XM_017008065; |
| | NM_182982; XM_011513457; XM_017008054; XM_024454015; XR_001741211; |
| | XR_924943; NM_001004057; NM_001350173; XM_006713880; XM_017008063; |
| | XM_011513450; XM_011513453; XM_011513454; XM_017008053; XM_017008056; |
| | XM_017008062; XM_017008066; XM_011513448; XM_011513451; XM_011513456; |
| | XM_017008057; XR_924941; NM_001004056; NM_005307; XM_017008055; |
| | XR_001741210 |
| GZMH | NM_001270781; XM_011536683; NM_033423; NM_001270780 |
| GZMA | NM_006144 |
| GZMB | NM_001346011; NM_004131; NR_144343 |
| GZMK | NM_002104 |
| HLA-A | XM_041680767; XR_005976896; NM_001242758; XM_041680768; NM_002116 |
| HLA-B | NM_005514 |
| HLA-C | NM_002117; NM_001243042 |
| HLA-DMA | NM_002118 |
| HLA-DPA1 | NM_001242525; NM_033554; NM_001242524 |
| HLA-DPB1 | NM_002121 |
| HLA-DQA1 | NM_002122; XM_006715079; |
| HLA-DQB1 | NM_001243962; NM_001243961; NM_002123 |
| HLA-DRA | NM_019111 |
| | XM_024452553; NM_001359194; XR_002958969; NM_001243965; NM_002124; |
| HLA-DRB1 | NM_001359193; XR_002958970 |
| TNC | XM_005251975; XM_006717096; XM_011518628; XM_017014681; XM_011518626; |
| | XM_005251973; XM_006717098; XM_005251972; XM_006717097; XM_011518629; |
| | XM_017014680; XM_011518625; XM_017014679; XM_005251974; XM_006717101; |
| | XM_017014678; XM_024447530; NM_002160 |
| IFNA2 | NM_000605 |
| IFNB1 | NM_002176 |
| IFNG | NM_000619 |
| IGF1R | XM_011521517; XM_024449913; NM_000875; XM_017022137; XM_011521516; |
| | XM_017022136; NM_152452; XM_017022138; XM_017022139; NM_001291858 |
| IGFBP4 | NM_001552 |
| IL1B | NM_000576; XM_017003988 |
| IL6 | NM_001318095; NM_000600; NM_001371096; XM_011515390; XM_005249745 |
| CXCL8 | NM_000584; NM_001354840 |
| CXCR1 | NM_000634 |
| CXCR2 | XM_017003990; XM_017003992; NM_001168298; NM_001557; XM_005246530; |
| | XM_017003991 |
| IL10 | NM_000572; NR_168467; NR_168466; NM_001382624 |
| IL12A | NM_000882; NM_001354583; NM_001354582; NM_001397992 |
| IL12B | NM_002187 |
| TNFRSF9 | NM_001561; XM_006710618 |
| IDO1 | NM_002164 |
| CXCL10 | NM_001565; NR_168520 |
| IRF5 | NM_001242452; XM_006715974; NM_001364314; NM_032643; XM_011516160; |
| | XM_011516158; NM_001347928; NM_001098629; XM_011516159; NM_001098627; |
| | NM_001098630 |
| ITK | NM_005546; XM_017009443 |
| KDR | NM_002253 |
| KIR2DL4 | NM_001080770; NM_001080772; NM_002255; NM_001258383 |
| KLRC2 | NM_002260 |
| LAG3 | NM_002286; XM_011520956 |
| LAMA3 | XM_011525981; XM_017025743; XR_001753199; NM_001127717; NM_000227; |
| | XM_011525978; XM_011525979; NM_198129; XM_011525980; XM_017025744; |
| | XM_011525982; NM_001302996; NR_130106; NM_001127718 |
| LAMB3 | XM_005273124; NM_001127641; XM_017001272; NM_000228; NM_001017402 |
| LAMC2 | NM_005562; NM_018891; XM_017001273 |
| LDHB | NM_001315537; NM_002300; XM_006719074; NM_001174097 |
| LGALS1 | NM_002305 |
| LGALS3 | NM_001357678; NR_003225; NM_002306; NM_001177388 |
| LGALS9 | XM_011524796; NM_001330163; NR_024043; XM_006721893; XM_006721895; |
| | NM_002308; XM_006721892; XM_017024623; NM_009587 |
| LIFR | XM_017009463; NM_001127671; NM_001364298; NM_002310; XM_017009462; |
| | XM_011514042; NM_001364297 |
| LOX | NM_001317073; NM_002317; NM_001178102 |
| LRP1 | NM_002332 |
| LUM | NM_002345 |
| MCM2 | NM_004526; XM_024453531; NR_073375 |
| MCM6 | NM_005915 |
| CIITA | XR_932842; NM_001379332; XM_006720880; XM_011522491; XR_932846; |
| | NM_001379334; XM_011522487; XR_932847; NM_001379333; XM_011522486; |
| | XM_011522494; XM_024450280; XR_932841; NM_000246; NM_001286402; |
| | XM_011522489; XM_024450281; XR_001751904; NM_001286403; NM_001379331; |
| | XM_011522485; NR_104444; XM_011522484; XM_011522490; NM_001379330 |
| MIF | NM_002415 |
| CXCL9 | NM_002416 |
| MKI67 | NM_002417; NM_001145966; XM_006717864; XM_011539818 |
| MMP1 | NM_001145938; NM_002421; |
| MMP2 | NM_001302509; NM_001127891; NM_001302508; NM_001302510; NM_004530 |
| MMP3 | NM_002422 |
| MMP7 | NM_002423 |
| MMP9 | NM_004994 |
| MMP11 | NM_005940; NR_133013 |
| MMP12 | NM_002426 |
| MPO | NM_000250 |
| MPP3 | XM_017024657; NM_001932; XM_017024660; XR_001752515; NR_003562; |
| | XM_006721916; NR_148344; XM_017024655; XM_017024658; XR_001752512; |
| | NR_148345; XM_006721915; XM_017024656; NR_148342; XM_017024659; |
| | XR_001752516; XR_001752517; XR_002958011; XR_934466; NM_001330233; |
| | NM_001353080; NR_148343 |
| MRC1 | NM_002438; NM_001009567 |
| MSR1 | NM_138716; NM_002445; XM_024447161; NM_138715; NM_001363744 |
| MYBL2 | NM_002466; NM_001278610 |
| NKG7 | XM_006723228; XM_005258955; NM_005601; NM_001363693 |
| NOS2 | NM_153292; NM_000625 |
| PAX5 | NM_001280547; NM_001280553; NM_016734; NM_001280548; NR_103999; |
| | NM_001280551; NM_001280555; NM_001280554; NM_001280552; NM_001280556; |
| | NM_001280549; NM_001280550; NR_104000 |
| PDCD1 | XM_017004293; NM_005018; XM_006712573 |
| PDGFRA | NM_001347828; NM_001347829; XM_005265743; XM_017008281; NM_001347827; |
| | NM_001347830; NM_006206; XM_006714041 |
| PDGFRB | NM_001355016; NM_002609; NM_001355017; NR_149150 |
| PGF | NM_002632; NM_001293643; NM_001207012 |
| PLK1 | NM_005030 |
| PLOD2 | XM_017006625; XM_024453599; NM_000935; NM_182943; XR_001740176; |
| | XM_005247535 |
| PLP1 | NM_001128834; NM_000533; NM_001305004; NM_199478 |
| PRF1 | NM_005041; NM_001083116 |
| PRTN3 | XM_011528136; NM_002777 |
| PTGS2 | NM_000963 |
| SCN2B | NM_004588 |
| CCL3 | NR_168496; NR_168495; NM_002983; NR_168494 |
| CXCL11 | NM_005409; NM_001302123 |
| CX3CL1 | NM_001304392; NM_002996 |
| CXCL12 | NM_000609; XR_001747171; XR_001747172; NM_199168; NM_001277990; |
| | XR_001747174; NM_001178134; XR_001747173; NM_001033886 |
| SLC1A2 | XM_017018138; XM_017018139; NM_001252652; XM_005253067; XM_011520285; |
| | XM_017018137; NM_001195728; XM_017018136; NM_004171 |
| SIGLEC1 | NM_001367089; NM_023068 |
| SOAT1 | NM_003101; NR_045530; XM_011509911; NM_001252511; XM_011509912; |
| | NM_001252512 |
| SOX4 | NM_003107 |
| AURKA | NM_001323304; NM_001323303; NM_198435; NM_198437; XM_024451974; |
| | NM_198433; NM_198434; NM_198436; XM_017028034; XM_017028035; |
| | NM_001323305; NM_003600 |
| TACC1 | XM_005273625; XM_005273629; NM_001352787; NM_001352795; NR_148051; |
| | XM_005273628; NM_001330521; NM_001352800; NM_001352802; NM_006283; |
| | XM_011544632; NM_001146216; NM_001352797; NM_001122824; NM_001352782; |
| | NM_001352801; NM_001352804; NR_148048; NM_001352779; NM_001352792; |
| | NM_001352796; NM_001352780; NM_001352781; NM_001352788; NM_001352790; |
| | NM_001352794; NR_148049; NR_148047; NR_148050; XM_011544636; |
| | NM_001352778; NM_001352784; NM_001352785; NM_001352786; NM_001352789; |
| | NM_001352791; NM_001352798; NM_001352799; XM_011544635; NM_001352783; |
| | NM_001352793; NM_001352803; NR_148052; NR_148053 |
| TAP1 | NM_000593; NM_001292022 |
| TAP2 | NM_001290043; NM_000544; NM_018833 |
| TAPBP | XM_017011227; XM_011514828; NM_003190; NM_172208; NM_172209 |
| TEK | NM_001375475; NM_000459; NM_001290077; NM_001290078; NM_001375476 |
| TGFB1 | NM_000660; XM_011527242 |
| TGFB2 | NM_003238; NR_138149; NR_138148; NM_001135599 |
| TGFB3 | NM_001329938; NM_003239; NM_001329939 |
| TNF | NM_000594 |
| TNFSF4 | XR_002957545; XM_017002230; XM_017002229; NM_003326; XR_001737393; |
| | XR_002957543; XR_001737394; NM_001297562; XM_017002228; XR_001737395; |
| | XM_011509964 |
| TNFRSF4 | XM_011542074; NM_003327; XM_017002232; XM_011542077; XM_011542075; |
| | XM_011542076; XM_017002231 |
| UTRN | XM_011536102; XM_017011245; XM_024446536; NM_007124; XM_005267133; |
| | XM_011536101; XM_017011243; XM_005267127; XM_005267130; XM_006715560; |
| | XM_017011244; NM_001375323 |
| VEGFA | NM_001171625; NM_003376; NM_001033756; NM_001171624; NM_001171626; |
| | NM_001171630; NM_001025366; NM_001317010; NM_001025368; NM_001025370; |
| | NM_001171623; NM_001171622; NM_001171628; NM_001171629; NM_001204385; |
| | NM_001025367; NM_001025369; NM_001171627; NM_001204384; NM_001287044 |
| VEGFB | NM_003377; NM_001243733 |
| VEGFC | NM_005429 |
| VTN | NM_000638 |
| VWF | NM_000552 |
| ZAP70 | XM_017004868; XR_001738927; NM_001378594; NM_207519; XM_017004869; |
| | NM_001079; XR_001738926; XM_017004870; XM_017004867; XR_001738925 |
| ZNF85 | XM_011528264; NM_001256172; NM_003429; NM_001256171; NM_001256173; |
| | XM_011528263; NR_045830 |
| ZNF141 | NM_001348277; NM_003441; NM_001348279; NM_001348278; XM_011513562; |
| | XM_017008591; NM_001348280 |
| MFAP5 | NM_001297709; NR_123733; NR_123734; NM_001297711; NM_003480; |
| | NM_001297710; NM_001297712 |
| AXIN2 | XM_011525319; XM_011525320; NM_001363813; XM_017025192; XM_017025193; |
| | XM_011525321; NM_004655 |
| EOMES | NM_001278182; XM_005265510; NM_005442; NM_001278183 |
| SORBS2 | XM_005263312; XM_017008740; XM_017008751; XM_017008760; XM_017008764; |
| | XM_017008770; NM_001145674; NM_001270771; NM_001394266; NM_001395207; |
| | NM_021069; XM_017008738; XM_017008741; XM_017008748; XM_017008754; |
| | XM_017008762; XM_017008765; XM_017008766; NM_001145671; NM_001394247; |
| | NM_001394252; NM_001394258; NM_001394262; NM_001394263; NM_001394274; |
| | NM_001394275; NM_001394277; XM_017008743; XM_017008755; XM_017008758; |
| | XM_017008768; XM_017008771; XM_024454258; NM_001145672; NM_001394245; |
| | NM_001394246; NM_001394257; NM_001394260; NM_001394265; NM_001394267; |
| | XM_005263308; XM_005263310; XM_017008753; XM_017008763; XM_017008772; |
| | XM_017008774; XM_024454260; NM_001145675; NM_001394264; NM_001394272; |
| | XM_005263311; XM_005263313; XM_017008739; XM_017008756; XM_017008767; |
| | NM_001145670; NM_001145673; NM_001394256; NM_001394268; NM_001394270; |
| | NM_001394271; XM_005263307; XM_017008757; NM_001394248; NM_001394254; |
| | NM_001394261; NM_003603; XM_006714390; XM_017008750; XM_017008752; |
| | XM_017008769; XM_017008775; NM_001394249; NM_001394250; NM_001394255; |
| | NM_001394259; XM_006714388; XM_017008744; XM_017008759; XM_017008761; |
| | XM_017008773; XM_024454257; XM_024454259; XR_002959769; NM_001394251; |
| | NM_001394253; NM_001394273; NM_001394276 |
| LGR5 | NR_110596; NM_001277227; NM_001277226; NM_003667 |
| TNFSF10 | NR_033994; NM_001190943; NM_003810; NM_001190942 |
| TNFSF9 | NM_003811 |
| TNFRSF18 | NM_148901; NM_004195; XM_017002722; NM_148902 |
| PGLYRP1 | NM_005091 |
| SOCS3 | NM_003955; NM_001378933; NM_001378932 |
| AURKB | NM_001313950; NM_001313953; XM_017025311; XM_017025307; XM_017025308; |
| | XM_017025309; NM_001313952; NM_004217; NM_001313954; NR_132730; |
| | NR_132731; XM_017025310; NM_001284526; XM_011524072; NM_001256834; |
| | NM_001313951; NM_001313955 |
| DLGAP1 | XM_005258173; XM_017026082; NM_001308390; NM_001398527; NM_001398532; |
| | XM_005258172; XM_006722367; XM_005258171; XM_011525771; NM_001242762; |
| | NM_001242765; NM_001398526; NM_001398535; NM_001398539; NM_001398540; |
| | XM_017026081; XM_017026080; XM_017026083; XM_017026084; XM_024451288; |
| | NM_001242763; NM_001242766; NM_004746; NM_001398530; NM_001398531; |
| | NM_001398537; NM_001398542; NM_001398543; XM_017026085; NM_001242764; |
| | NM_001398541; NM_001398546; XM_011525770; NM_001003809; NM_001242761; |
| | NM_001398525; NM_001398528; NM_001398533; NM_001398545; XM_005258174; |
| | NM_001398534; NM_001398536; NM_001398544 |
| CD83 | NM_001040280; NM_001251901; NM_004233 |
| CD163 | XR_002957389; XM_024449278; NM_203416; NM_001370145; NM_001370146; |
| | NM_004244; NR_163255 |
| SLIT2 | NM_004787; XM_011513909; XM_005248211; XM_017008845; XM_011513910; |
| | NM_001289136; XM_006713986; NM_001289135 |
| NCR1 | XM_011527528; NM_004829; XM_011527530; NM_001145457; NM_001242357; |
| | XM_011527529; XR_001753801; NM_001242356; NM_001145458 |
| ADAMTS4 | XR_001737548; NM_001320336; NM_005099; XR_001737549 |
| ARNT2 | NM_014862 |
| CAP2 | NM_001363534; NM_006366; NM_001363533 |
| GNLY | XM_005264085; NM_001302758; XM_005264087; NM_006433; XM_005264084; |
| | NM_012483 |
| CXCR6 | NM_001386435; NM_001386436; NM_006564; NM_001386437 |
| CD226 | XM_017025527; NM_006566; NM_001303619; XM_017025525; XM_006722374; |
| | XM_017025526; XM_005266642; NM_001303618; XM_005266643 |
| ZNF273 | NM_001385647; NR_169738; NR_169743; NR_169744; NM_001385643; |
| | XM_024446635; NM_001385650; NM_021148; NR_169745; NR_169741; |
| | XM_024446640; NR_003099; NM_001385645; NM_001385649; NR_169747; |
| | NR_169748; NR_170871; XM_024446636; NM_001385644; NR_169746; |
| | NM_033548; NM_001385646; NR_169742; NM_001385652; NM_001388021 |
| ADAMTS5 | XM_024452053; XM_024452054; NM_007038 |
| CD160 | NM_007053; XM_005272929; XM_011509104; NR_103845 |
| FSTL1 | XM_024453327; NM_007085 |
| IKZF2 | XM_011510804; XM_011510815; NM_001371277; XM_011510803; XM_011510808; |
| | XM_011510816; NM_001371275; XM_005246386; XM_011510807; XM_011510810; |
| | XM_011510811; NM_001371276; XM_005246385; XM_011510809; NM_001079526; |
| | NM_001371274; NM_001387220; NM_016260; XM_005246384; XM_011510817; |
| | XM_011510818; XM_017003591; XM_011510805; XM_011510812; XM_011510819; |
| | XM_017003592; XM_011510802 |
| SEPT6 | XR_001755675; NM_015129; XM_011531318; NM_145800; NM_145802; |
| | XM_006724748; XR_001755676; XR_001755677; NM_145799; XM_011531317; |
| | XM_006724750; XM_005262400 |
| ICOSLG | NM_001395918; NM_001283050; XM_011529514; NM_001283051; NM_001283052; |
| | NM_015259; XM_024452060; NM_001365759; XM_011529516 |
| TNFRSF13B | NM_012452 |
| ABTB2 | NM_145804 |
| PITPNC1 | XM_017024443; NM_181671; XM_005257216; XM_017024442; XM_017024445; |
| | NM_012417; XM_017024444 |
| STAP1 | NM_012108; NM_001317769; XM_017008018 |
| SLCO3A1 | XM_005254889; XR_931795; XM_005254891; XR_931796; NM_001145044; |
| | XM_011521456; NM_013272; NR_135775 |
| CD274 | NM_001314029; NR_052005; NM_001267706; NM_014143 |
| ICOS | NM_012092 |
| MDFIC | NM_199072; NM_001166345; NM_001166346 |
| TBX21 | NM_013351 |
| IL22 | NM_020525 |
| ARHGEF4 | NM_001375902; NM_001375900; NM_001375901; NM_001375904; NM_001367493; |
| | NM_001375903; NM_015320; NM_001395416; NM_032995 |
| TRAT1 | NM_016388; NM_001317747 |
| FOXP3 | XM_006724533; NM_001114377; NM_014009; XM_017029567 |
| KLRF1 | XM_017019415; NM_001291822; XR_931301; NM_001366534; NR_120305; |
| | NM_001291823; NM_016523; NR_159359; NR_159360; NR_159361 |
| DTL | XM_011509614; NM_001286229; NM_001286230; NM_016448 |
| IL23A | XM_011538477; NM_016584 |
| CD244 | NM_001166663; XR_001737229; XM_011509622; NM_016382; NM_001166664; |
| | XM_011509623; XM_011509621 |
| IL17RD | NM_001318864; NM_017563; XM_011533849; XM_005265238 |
| KIF26B | XM_017030182; XM_017030183; NM_018012 |
| SLC38A4 | NM_018018; XM_005268997; NM_001143824 |
| TNFRSF19 | XM_005266445; NM_001354985; NM_148957; XM_017020651; NM_001204458; |
| | NM_018647; XM_011535146; NM_001204459; XM_005266446 |
| BEX1 | NM_018476 |
| PDGFC | XM_011532124; XM_017008456; XM_017008455; NM_016205; NR_036641 |
| SERTAD4 | NM_001354173; XR_921894; NM_001375428; NM_019605 |
| CD248 | NM_020404 |
| CD177 | XM_017027021; XM_017027022; NM_020406 |
| GRAMD1A | XM_011527153; XM_017027035; XM_011527155; XM_024451622; XM_024451623; |
| | NM_001320035; NM_020895; XM_011527149; XM_011527156; NM_001136199; |
| | NM_001320036; XM_011527154; XM_017027034; NM_001320034 |
| CXCL16 | NM_001100812; NM_001386809; NM_022059 |
| IL21 | NM_021803; NM_001207006 |
| VSIR | NM_022153 |
| IKZF4 | XM_017019810; XM_017019814; XM_017019813; XM_017019809; NM_001351089; |
| | NM_022465; XM_017019807; XM_017019815; XM_017019816; XM_024449130; |
| | NM_001351090; NM_001351091; XM_011538664; XM_011538669; XM_017019806; |
| | XM_017019808; XM_017019811; XM_005269086; XM_005269089; XM_024449128; |
| | XM_024449129; XM_024449131; NM_001351092; XM_017019812 |
| DYNC2H1 | XM_006718903; NM_001377; NM_024606; NM_001080463; XM_017018291; |
| | XM_017018292; XM_017018293 |
| PDCD1LG2 | XM_005251600; NM_025239 |
| ZNF93 | NM_031218; NM_001004126 |
| FCRL5 | XM_011510032; XM_011510030; XM_011510031; XM_011510033; NM_031281; |
| | NM_001195388 |
| FGFBP2 | NM_031950 |
| RASSF4 | NM_178145; NM_032023 |
| NLRC5 | XM_005256196; XM_011523373; XR_001751997; NM_001330552; NM_001384961; |
| | NM_001384969; NM_001384972; NM_001384973; NR_169518; XM_005256201; |
| | XR_001751999; XR_001752000; NM_001384951; NM_001384959; NM_032206; |
| | NR_169513; XM_006721300; XM_017023770; NM_001384950; NM_001384958; |
| | NM_001384964; NR_169520; XM_017023771; XR_001751998; NM_001384954; |
| | NM_001384966; NR_169514; NR_169517; NM_001384967; NM_001384971; |
| | XM_006721298; XR_429734; NM_001384965; NM_001384970; NR_169512; |
| | NR_169519; XM_005256194; XM_011523376; NM_001384953; NM_001384956; |
| | NM_001384957; NM_001384962; NM_001384963; NR_169515; NR_169516; |
| | NM_001384952; NM_001384955; NM_001384960; NM_001384968 |
| BEX2 | NM_001168400; NM_001168399; NM_001168401; NM_032621 |
| HAVCR2 | NM_032782 |
| KLHL13 | NM_001168300; NM_001394866; NM_001394863; NM_033495; XM_011531410; |
| | NM_001168301; NM_001168302; XM_011531409; NM_001394865; XM_017029950; |
| | NM_001168299; NM_001394864; NM_001168303 |
| KIF12 | XR_002956749; NM_001388308; XM_024447405; XM_024447406; XR_002956751; |
| | XM_005251683; XM_006716947; XM_024447407; XR_002956750; NM_138424 |
| FMNL2 | NM_052905; XM_011510532; XM_011510533; XM_011510534; XM_011510536; |
| | NM_001004417; XM_011510535; XR_241279; NM_001004422; XM_005246263; |
| | XM_011510530; NM_001004421; XM_011510531; XM_005246265 |
| RNF157 | XM_005257007; NM_001330501; XM_017024120; XM_017024117; XR_001752422; |
| | XM_011524273; NM_052916 |
| TNFRSF13C | NM_052945 |
| SH2D1B | NM_053282 |
| TMEM182 | XM_011510632; XM_017003376; XM_017003375; NM_144632; XM_006712288; |
| | NM_001321344; NM_001321345; XM_006712287; XR_427070; NM_001321343; |
| | NM_001321346 |
| AFAP1L1 | XM_011537558; NM_001323062; NM_001323063; XM_017009036; NM_001146337; |
| | NM_152406 |
| APCDD1 | NM_153000; XM_011525603 |
| ZNF714 | NR_117088; NR_117087; NR_117086; NM_182515 |
| BTLA | NM_001085357; NM_181780; XM_011512447; XM_017005748 |
| ESCO2 | NM_001017420; XR_949378; XM_011544422; XM_011544421 |
| ZNF92 | NM_001287533; NM_007139; NM_152626; NM_001287534; NM_001287532 |
| ZNF626 | NM_145297; NM_001076675 |
| TIGIT | XR_002959502; NM_173799; XM_024453388 |
| NPNT | NM_198278; NM_001184693; XM_011531822; XM_011531825; XM_011531820; |
| | XM_017007984; NM_001184691; NM_001184692; XM_011531824; XM_011531823; |
| | XM_005262888; NM_001033047; NM_001184690 |
| NCR3 | NM_001145467; XM_011514459; XM_006715049; NM_001145466; NM_147130 |
| IL4I1 | NM_001385639; NM_172374; NM_152899; NR_047577; NM_001258018; |
| | NM_001258017 |
| ZNF493 | NM_145326; NM_175910; NM_001076678 |
| ZNF680 | XR_428175; XM_024446744; XM_024446747; XR_001744687; XM_024446745; |
| | NM_001130022; NM_178558; XM_024446742; XM_006715959; XM_024446748; |
| | XM_024446743; XM_024446746; XR_002956428; XM_017012126; XM_017012127; |
| | XM_017012129; XM_017012128; XR_002956429 |
| CCDC46 | NR_126542.2; XR_001752444.1; XR_934412.2; XR_934414.2; NM_001353128.2; |
| | NM_001199165.4; NM_001353129.2; NM_001302891.3; NM_001353127.2; |
| | NM_001037325.3; XM_011524464.3; XM_005257119.5; XM_024450634.1; |
| | XM_011524465.2; XM_005257125.3; XM_011524466.2; XM_011524467.2; |
| | XM_005257126.4; XM_011524463.2; XM_011524462.3; XM_006721744.3 |
| CCL4 | NM_002984.4 |
| CXCL5 | NM_002994.5 |
| ELN | XM_011515870.1; XM_011515874.1; XM_011515875.2; XM_011515868.2; |
| | XM_011515876.2; XM_011515873.2; XM_011515872.2; XM_011515877.2; |
| | XM_011515871.2; XM_017011814.2; XM_005250187.2; XM_011515869.1; |
| | NM_001081754.3; NM_001081753.3; NM_001278939.2; NM_001278915.2; |
| | NM_000501.4; NM_001278912.2; NM_001081755.3; XM_005250188.2; |
| | NM_001278916.2; NM_001278913.2; NM_001278914.2; NM_001278917.2 |
| | NM_001081752.3; XM_017011813.1; NM_001278918.2 |
| FAM64A | NM_019013.3; NM_001195228.2; XM_017024778.1 |
| HLA-DMB | NM_002118.5 |
| KLRK1 | NM_007360.4 |
| LGALS7 | NM_002307.4 |
| LOC285141 | NR_110644.1; XM_017003875.1; NM_001388469.1; NM_001290030.2; |
| | NM_001388471.1; NM_001388470.1; NM_001388468.1; NM_001388467.1; |
| | NM_001290031.1; XM_011511001.2; NM_001289947.2 |
| TRAC | X02592.1 |
| TRBC1 | BC030533.1 |
| TRBC2 | ENSG00000211772 |
| ZNF678 | NR_102302.2, NM_001367910.1, NM_178549.4, NM_001367909.1, NM_001367911.1 |

### EQUIVALENTS

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for identifying a gastric cancer (GC) tumor microenvironment (TME) type for a subject, the method comprising:
using at least one computer hardware processor to perform:
(a) obtaining RNA expression data for the subject, the RNA expression data indicating RNA expression levels for at least some genes in each group of at least some of a plurality of gene groups listed in Table 1;
(b) generating a GC TME signature for the subject using the RNA expression data, the GC TME signature comprising gene group scores for respective gene groups in the at least some of the plurality of gene groups, the generating comprising:
determining the gene group scores using the RNA expression levels; and
(c) identifying, using the GC TME signature and from among a plurality of GC TME types, a GC TME type for the subject.

2. The method of claim 1, wherein the subject has, is suspected of having, or is at risk of having gastric cancer.

3. The method of claim 1, further comprising:
identifying the subject as having GC TME type E; and
when the subject is identified as having the GC TME type E, administering an immunotherapy to the subject.

4. The method of claim 1, wherein obtaining the RNA expression data for the subject comprises obtaining sequencing RNA data previously obtained by sequencing a biological sample obtained from the subject.

5. The method of claim 4, wherein the sequencing data comprises at least 1 million reads, at least 5 million reads, at least 10 million reads, at least 20 million reads, at least 50 million reads, or at least 100 million reads.

6. The method of claim 4, wherein the sequencing data comprises whole exome sequencing (WES) data, bulk RNA sequencing (RNA-seq) data, single cell RNA sequencing (scRNA-seq) data, or next generation sequencing (NGS) data.

7. The method of claim 4, wherein the sequencing data comprises microarray data.

8. The method of claim 1, further comprising:
normalizing the RNA expression data to transcripts per million (TPM) units prior to generating the GC TME signature.

9. The method of claim 1, wherein obtaining the RNA expression data for the subject comprises sequencing a biological sample obtained from the subject.

10. The method of claim 9, wherein the biological sample comprises gastrointestinal tissue of the subject, optionally wherein the biological sample comprises tumor tissue of the subject.

11. The method of claim 1, wherein the RNA expression levels comprise RNA expression levels for at least three genes from each of at least two of the following gene groups:
(i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(ii) T cells group: TBX21, ITK, CD3D, CD3E, CD3G, TRAC, TRBC1, TRBC2, CD28, CD5, TRAT1;
(iii) B cells group: CD19, MS4A1, TNFRSF13C, CR2, TNFRSF17, TNFRSF13B, CD22, CD79A, CD79B, BLK, FCRL5, PAX5, STAP1;
(iv) Treg group: FOXP3, CTLA4, IL10, TNFRSF18, CCR8, IKZF4, IKZF2;
(v) MDSC group: IDO1, ARG1, IL10, CYBB, PTGS2, IL4I1, IL6;
(vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(vii) Proliferation rate group: MKI67, ESCO2, CETN3, CDK2, CCND1, CCNE1, AURKA, AURKB, E2F1, MYBL2, BUB1, CCNB1, MCM2, MCM6; and
(viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

12. The method of claim 1, wherein the RNA expression levels comprise RNA expression levels for each of the genes from each of the following gene groups:
(i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(ii) T cells group: TBX21, ITK, CD3D, CD3E, CD3G, TRAC, TRBC1, TRBC2, CD28, CD5, TRAT1;
(iii) B cells group: CD19, MS4A1, TNFRSF13C, CR2, TNFRSF17, TNFRSF13B, CD22, CD79A, CD79B, BLK, FCRL5, PAX5, STAP1;
(iv) Treg group: FOXP3, CTLA4, IL10, TNFRSF18, CCR8, IKZF4, IKZF2;
(v) MDSC group: IDO1, ARG1, IL10, CYBB, PTGS2, IL4I1, IL6;
(vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(vii) Proliferation rate group: MKI67, ESCO2, CETN3, CDK2, CCND1, CCNE1, AURKA, AURKB, E2F1, MYBL2, BUB1, CCNB1, MCM2, MCM6; and
(viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

13. The method of claim 1, wherein the RNA expression levels comprise RNA expression levels for at least three genes from each of at least two of the following gene groups:
(a) MHC I group: HLA-A, HLA-B, HLA-C, B2M, TAP1, TAP2, NLRC5, TAPBP;
(b) MHC II group: HLA-DRA, HLA-DRB1, HLA-DMA, HLA-DPA1, HLA-DPB1, HLA-DMB, HLA-DQB1, HLA-DQA1, CIITA;
(c) Coactivation molecules group: CD28, CD40, TNFRSF4, ICOS, TNFRSF9, CD27, CD80, CD86, CD40LG, CD83, TNFSF4, ICOSLG, TNFSF9, CD70;
(d) Effector cells group: IFNG, GZMA, GZMB, PRF1, GZMK, ZAP70, GNLY, FASLG, TBX21, EOMES, CD8A, CD8B;
(e) T cell traffic group: CXCL9, CXCL10, CXCL11, CX3CL1, CCL3, CCL4, CX3CR1, CXCL16, CXCR6;
(f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(g) T cells group: TBX21, ITK, CD3D, CD3E, CD3G, TRAC, TRBC1, TRBC2, CD28, CD5, TRAT1;
(h) B cells group: CD19, MS4A1, TNFRSF13C, CR2, TNFRSF17, TNFRSF13B, CD22, CD79A, CD79B, BLK, FCRL5, PAX5, STAP1;
(i) M1 signature group: NOS2, TNF, IL1B, SOCS3, CMKLR1, IRF5, IL12A, IL12B, IL23A;
(j) Antitumor cytokines group: TNF, IFNB1, IFNA2, CCL3, TNFSF10, IL21;
(k) Checkpoint inhibition group: PDCD1, CD274, CTLA4, LAG3, PDCD1LG2, BTLA, HAVCR2, TIGIT, VSIR;
(l) Treg group: FOXP3, CTLA4, IL10, TNFRSF18, CCR8, IKZF4, IKZF2;
(m) Neutrophil signature group: MPO, ELANE, PRTN3, CTSG, CXCR1, CXCR2, FCGR3B, CD177, FFAR2, PGLYRP1;
(n) MDSC group: IDO1, ARG1, IL10, CYBB, PTGS2, IL4I1, IL6;
(o) M2 signature group: IL10, MRC1, MSR1, CD163, CSF1R, IL4I1, SIGLEC1, CD68;
(p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(q) Angiogenesis group: VEGFA, VEGFB, VEGFC, PDGFC, CXCL8, CXCR2, FLT1, PGF, KDR, ANGPT1, ANGPT2, TEK, VWF, CDH5;
(r) Proliferation rate group: MKI67, ESCO2, CETN3, CDK2, CCND1, CCNE1, AURKA, AURKB, E2F1, MYBL2, BUB1, CCNB1, MCM2, MCM6; and
(s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

14. The method of claim 1, wherein determining the gene group scores comprises:
determining a respective gene group score for each of at least two of the following gene groups, using, for a particular gene group, RNA expression levels for at least three genes in the particular gene group to determine the gene group score for the particular group, the gene groups including:
(i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(ii) T cells group: TBX21, ITK, CD3D, CD3E, CD3G, TRAC, TRBC1, TRBC2, CD28, CD5, TRAT1;
(iii) B cells group: CD19, MS4A1, TNFRSF13C, CR2, TNFRSF17, TNFRSF13B, CD22, CD79A, CD79B, BLK, FCRL5, PAX5, STAP1;
(iv) Treg group: FOXP3, CTLA4, IL10, TNFRSF18, CCR8, IKZF4, IKZF2;
(v) MDSC group: IDO1, ARG1, ILO, CYBB, PTGS2, IL4I1, IL6;
(vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(vii) Proliferation rate group: MKI67, ESCO2, CETN3, CDK2, CCND1, CCNE1, AURKA, AURKB, E2F1, MYBL2, BUB1, CCNB1, MCM2, MCM6; and
(viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

15. The method of claim 1, wherein determining the gene group scores comprises:
determining a respective gene group score for each of the following gene groups, using, for each gene group, RNA expression levels for each of the genes in each gene group to determine the gene group score for each particular group, the gene groups including:
(i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(ii) T cells group: TBX21, ITK, CD3D, CD3E, CD3G, TRAC, TRBC1, TRBC2, CD28, CD5, TRAT1;
(iii) B cells group: CD19, MS4A1, TNFRSF13C, CR2, TNFRSF17, TNFRSF13B, CD22, CD79A, CD79B, BLK, FCRL5, PAX5, STAP1;
(iv) Treg group: FOXP3, CTLA4, IL10, TNFRSF18, CCR8, IKZF4, IKZF2;
(v) MDSC group: IDO1, ARG1, IL10, CYBB, PTGS2, IL4I1, IL6;
(vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(vii) Proliferation rate group: MKI67, ESCO2, CETN3, CDK2, CCND1, CCNE1, AURKA, AURKB, E2F1, MYBL2, BUB1, CCNB1, MCM2, MCM6; and
(viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

16. The method of claim 15, wherein determining the gene group scores comprises determining a first score of a first gene group using a single-sample GSEA (ssGSEA) technique from RNA expression levels for at least some of the genes in one of the following gene groups:
(i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(ii) T cells group: TBX21, ITK, CD3D, CD3E, CD3G, TRAC, TRBC1, TRBC2, CD28, CD5, TRAT1;
(iii) B cells group: CD19, MS4A1, TNFRSF13C, CR2, TNFRSF17, TNFRSF13B, CD22, CD79A, CD79B, BLK, FCRL5, PAX5, STAP1;
(iv) Treg group: FOXP3, CTLA4, IL10, TNFRSF18, CCR8, IKZF4, IKZF2;
(v) MDSC group: IDO1, ARG1, IL10, CYBB, PTGS2, IL4I1, IL6;
(vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(vii) Proliferation rate group: MKI67, ESCO2, CETN3, CDK2, CCND1, CCNE1, AURKA, AURKB, E2F1, MYBL2, BUB1, CCNB1, MCM2, MCM6; and
(viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

17. The method of claim 1, wherein determining the gene group scores comprises determining the gene group scores, using a single-sample GSEA (ssGSEA) technique, from RNA expression levels for each of the genes in each of the following gene groups:
(i) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(ii) T cells group: TBX21, ITK, CD3D, CD3E, CD3G, TRAC, TRBC1, TRBC2, CD28, CD5, TRAT1;
(iii) B cells group: CD19, MS4A1, TNFRSF13C, CR2, TNFRSF17, TNFRSF13B, CD22, CD79A, CD79B, BLK, FCRL5, PAX5, STAP1;
(iv) Treg group: FOXP3, CTLA4, IL10, TNFRSF18, CCR8, IKZF4, IKZF2;
(v) MDSC group: IDO1, ARG1, IL10, CYBB, PTGS2, IL4I1, IL6;
(vi) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(vii) Proliferation rate group: MKI67, ESCO2, CETN3, CDK2, CCND1, CCNE1, AURKA, AURKB, E2F1, MYBL2, BUB1, CCNB1, MCM2, MCM6; and
(viii) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

18. The method of claim 1, wherein determining the gene group scores comprises:
determining a respective gene group score for each of at least two of the following gene groups, using, for a particular gene group, RNA expression levels for at least three genes in the particular gene group to determine the gene group score for the particular group, the gene groups including:
(a) MHC I group: HLA-A, HLA-B, HLA-C, B2M, TAP1, TAP2, NLRC5, TAPBP;
(b) MHC II group: HLA-DRA, HLA-DRB1, HLA-DMA, HLA-DPA1, HLA-DPB1, HLA-DMB, HLA-DQB1, HLA-DQA1, CIITA;
(c) Coactivation molecules group: CD28, CD40, TNFRSF4, ICOS, TNFRSF9, CD27, CD80, CD86, CD40LG, CD83, TNFSF4, ICOSLG, TNFSF9, CD70;
(d) Effector cells group: IFNG, GZMA, GZMB, PRF1, GZMK, ZAP70, GNLY, FASLG, TBX21, EOMES, CD8A, CD8B;
(e) T cell traffic group: CXCL9, CXCL10, CXCL11, CX3CL1, CCL3, CCL4, CX3CR1, CXCL16, CXCR6;
(f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(g) T cells group: TBX21, ITK, CD3D, CD3E, CD3G, TRAC, TRAC, TRBC2, CD28, CD5, TRAT1;
(h) B cells group: CD19, MS4A1, TNFRSF13C, CR2, TNFRSF17, TNFRSF13B, CD22, CD79A, CD79B, BLK, FCRL5, PAX5, STAP1;
(i) M1 signature group: NOS2, TNF, IL1B, SOCS3, CMKLR1, IRF5, IL12A, IL12B, IL23A;
(j) Antitumor cytokines group: TNF, IFNB1, IFNA2, CCL3, TNFSF10, IL21;
(k) Checkpoint inhibition group: PDCD1, CD274, CTLA4, LAG3, PDCD1LG2, BTLA, HAVCR2, TIGIT, VSIR;
(l) Treg group: FOXP3, CTLA4, IL10, TNFRSF18, CCR8, IKZF4, IKZF2;
(m) Neutrophil signature group: MPO, ELANE, PRTN3, CTSG, CXCR1, CXCR2, FCGR3B, CD177, FFAR2, PGLYRP1;
(n) MDSC group: IDO1, ARG1, ILO, CYBB, PTGS2, IL4I1, IL6;
(o) M2 signature group: IL10, MRC1, MSR1, CD163, CSF1R, IL4I1, SIGLEC1, CD68;
(p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(q) Angiogenesis group: VEGFA, VEGFB, VEGFC, PDGFC, CXCL8, CXCR2, FLT1, PGF, KDR, ANGPT1, ANGPT2, TEK, VWF, CDH5;
(r) Proliferation rate group: MKI67, ESCO2, CETN3, CDK2, CCND1, CCNE1, AURKA, AURKB, E2F1, MYBL2, BUB1, CCNB1, MCM2, MCM6; and
(s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

19. The method of claim 18, wherein determining the gene group scores comprises determining the gene group scores, using a single-sample GSEA (ssGSEA) technique, from RNA expression levels for at least some of the genes in each one of the following gene groups:
(a) MHC I group: HLA-A, HLA-B, HLA-C, B2M, TAP1, TAP2, NLRC5, TAPBP;
(b) MHC II group: HLA-DRA, HLA-DRB1, HLA-DMA, HLA-DPA1, HLA-DPB1, HLA-DMB, HLA-DQB1, HLA-DQA1, CIITA;
(c) Coactivation molecules group: CD28, CD40, TNFRSF4, ICOS, TNFRSF9, CD27, CD80, CD86, CD40LG, CD83, TNFSF4, ICOSLG, TNFSF9, CD70;
(d) Effector cells group: IFNG, GZMA, GZMB, PRF1, GZMK, ZAP70, GNLY, FASLG, TBX21, EOMES, CD8A, CD8B;
(e) T cell traffic group: CXCL9, CXCL10, CXCL11, CX3CL1, CCL3, CCL4, CX3CR1, CXCL16, CXCR6;
(f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(g) T cells group: TBX21, ITK, CD3D, CD3E, CD3G, TRAC, TRBC1, TRBC2, CD28, CD5, TRAT1;
(h) B cells group: CD19, MS4A1, TNFRSF13C, CR2, TNFRSF17, TNFRSF13B, CD22, CD79A, CD79B, BLK, FCRL5, PAX5, STAP1;
(i) M1 signature group: NOS2, TNF, IL1B, SOCS3, CMKLR1, IRF5, IL12A, IL12B, IL23A;
(j) Antitumor cytokines group: TNF, IFNB1, IFNA2, CCL3, TNFSF10, IL21;
(k) Checkpoint inhibition group: PDCD1, CD274, CTLA4, LAG3, PDCD1LG2, BTLA, HAVCR2, TIGIT, VSIR;
(l) Treg group: FOXP3, CTLA4, IL10, TNFRSF18, CCR8, IKZF4, IKZF2;
(m) Neutrophil signature group: MPO, ELANE, PRTN3, CTSG, CXCR1, CXCR2, FCGR3B, CD177, FFAR2, PGLYRP1;
(n) MDSC group: IDO1, ARG1, IL10, CYBB, PTGS2, IL4I1, IL6;
(o) M2 signature group: IL10, MRC1, MSR1, CD163, CSF1R, IL4I1, SIGLEC1, CD68;
(p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(q) Angiogenesis group: VEGFA, VEGFB, VEGFC, PDGFC, CXCL8, CXCR2, FLT1, PGF, KDR, ANGPT1, ANGPT2, TEK, VWF, CDH5;
(r) Proliferation rate group: MKI67, ESCO2, CETN3, CDK2, CCND1, CCNE1, AURKA, AURKB, E2F1, MYBL2, BUB1, CCNB1, MCM2, MCM6; and
(s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

20. The method of claim 1, wherein determining the gene group scores comprises determining the gene group scores, using a single-sample GSEA (ssGSEA) technique, from RNA expression levels for each of the genes in each of the following gene groups:
(a) MHC I group: HLA-A, HLA-B, HLA-C, B2M, TAP1, TAP2, NLRC5, TAPBP;
(b) MHC II group: HLA-DRA, HLA-DRB1, HLA-DMA, HLA-DPA1, HLA-DPB1, HLA-DMB, HLA-DQB1, HLA-DQA1, CIITA;
(c) Coactivation molecules group: CD28, CD40, TNFRSF4, ICOS, TNFRSF9, CD27, CD80, CD86, CD40LG, CD83, TNFSF4, ICOSLG, TNFSF9, CD70;
(d) Effector cells group: IFNG, GZMA, GZMB, PRF1, GZMK, ZAP70, GNLY, FASLG, TBX21, EOMES, CD8A, CD8B;
(e) T cell traffic group: CXCL9, CXCL10, CXCL11, CX3CL1, CCL3, CCL4, CX3CR1, CXCL16, CXCR6;
(f) NK cells group: NKG7, CD160, CD244, NCR1, KLRC2, KLRK1, CD226, GZMH, GNLY, IFNG, KIR2DL4, EOMES, GZMB, FGFBP2, KLRF1, SH2D1B, NCR3;
(g) T cells group: TBX21, ITK, CD3D, CD3E, CD3G, TRAC, TRBC1, TRBC2, CD28, CD5, TRAT1;
(h) B cells group: CD19, MS4A1, TNFRSF13C, CR2, TNFRSF17, TNFRSF13B, CD22, CD79A, CD79B, BLK, FCRL5, PAX5, STAP1;
(i) M1 signature group: NOS2, TNF, IL1B, SOCS3, CMKLR1, IRF5, IL12A, IL12B, IL23A;
(j) Antitumor cytokines group: TNF, IFNB1, IFNA2, CCL3, TNFSF10, IL21;
(k) Checkpoint inhibition group: PDCD1, CD274, CTLA4, LAG3, PDCD1LG2, BTLA, HAVCR2, TIGIT, VSIR;
(l) Treg group: FOXP3, CTLA4, IL10, TNFRSF18, CCR8, IKZF4, IKZF2;
(m) Neutrophil signature group: MPO, ELANE, PRTN3, CTSG, CXCR1, CXCR2, FCGR3B, CD177, FFAR2, PGLYRP1;
(n) MDSC group: IDO1, ARG1, IL10, CYBB, PTGS2, IL4I1, IL6;
(o) M2 signature group: IL10, MRC1, MSR1, CD163, CSF1R, IL4I1, SIGLEC1, CD68;
(p) Cancer associated fibroblast (CAF) group: LGALS1, COL1A1, COL1A2, COL5A1, ACTA2, FAP, LRP1, CD248, COL6A1, COL6A2, COL6A3, COL11A1, CXCL12, FBLN1, LUM, MFAP5, MMP3, MMP2, PDGFRB, PDGFRA, FN1, COL1A1, COL1A2, COL4A1, COL3A1, VTN, LGALS7, LGALS9, LAMA3, LAMB3, LAMC2, TNC, COL5A1, COL11A1, LGALS3, CA9, MMP9, MMP2, MMP1, MMP3, MMP12, MMP7, MMP11, PLOD2, ADAMTS4, ADAMTS5, LOX;
(q) Angiogenesis group: VEGFA, VEGFB, VEGFC, PDGFC, CXCL8, CXCR2, FLT1, PGF, KDR, ANGPT1, ANGPT2, TEK, VWF, CDH5;
(r) Proliferation rate group: MKI67, ESCO2, CETN3, CDK2, CCND1, CCNE1, AURKA, AURKB, E2F1, MYBL2, BUB1, CCNB1, MCM2, MCM6; and
(s) Lgr5 ISC group: ABTB2, AFAP1L1, APCDD1, ARHGEF4, ARNT2, AXIN2, BCL2, BEX1, BEX2, CAP2, CCDC46, CYP2E1, DGKG, DLGAP1, DTL, DYNC2H1, EPHA4, FAM64A, FGFR4, FMNL2, FSTL1, GRAMD1A, GRK4, IGF1R, IGFBP4, IL17RD, KIF12, KIF26B, KLHL13, LDHB, LGR5, LIFR, LOC285141, MDFIC, MPP3, NPNT, PITPNC1, PLP1, RASSF4, RNF157, SCN2B, SEPT6, SERTAD4, SLC1A2, SLC38A4, SLCO3A1, SLIT2, SOAT1, SORBS2, SOX4, TACC1, TMEM182, TNFRSF19, UTRN, ZNF141, ZNF273, ZNF493, ZNF626, ZNF678, ZNF680, ZNF714, ZNF85, ZNF92, ZNF93.

21. The method of claim 1, wherein generating the GC TME signature further comprises normalizing the gene group scores, wherein the normalizing comprises applying rank estimation and/or median scaling to the gene group scores.

22. The method of claim 1, wherein the plurality of GC TME types is associated with a respective plurality of GC TME signature clusters,
wherein identifying, using the GC TME signature and from among a plurality of GC TME types, the GC TME type for the subject comprises:
associating the GC TME signature of the subject with a particular one of the plurality of GC TME signature clusters; and
identifying the GC TME type for the subject as the GC TME type corresponding to the particular one of the plurality of GC TME signature clusters to which the GC TME signature of the subject is associated.

23. The method of claim 22, further comprising generating the plurality of GC TME signature clusters, the generating comprising:
obtaining multiple sets of RNA expression data by sequencing biological samples from multiple respective subjects, each of the multiple sets of RNA expression data indicating RNA expression levels for at least some genes in each of the at least some of the plurality of gene groups listed in Table 1;
generating multiple GC TME signatures from the multiple sets of RNA expression data, each of the multiple GC TME signatures comprising gene group scores for respective gene groups in the plurality of gene groups, the generating comprising,
for each particular one of the multiple GC TME signatures, determining the GC TME signature by determining the gene group scores using the RNA expression levels in the particular set of RNA expression data for which the particular one GC TME signature is being generated; and
clustering the multiple GC signatures to obtain the plurality of GC TME signature clusters.

24. The method of claim 23, wherein the clustering comprises dense clustering, spectral clustering, k-means clustering, hierarchical clustering, and/or an agglomerative clustering.

25. The method of claim 24, wherein the hierarchical clustering is performed using a Louvain community detection algorithm.

26. The method of claim 23, further comprising:
updating the plurality of GC TME signature clusters using the GC TME signature of the subject, wherein the GC TME signature of the subject is one of a threshold number GC TME signatures for a threshold number of subjects, wherein when the threshold number of GC TME signatures is generated the GC TME signature clusters are updated.

27. The method of claim 26, wherein the threshold number of GC TME signatures is at least 50, at least 75, at least 100, at least 200, at least 500, at least 1000, or at least 5000 GC TME signatures.

28. The method of claim 26, wherein the updating comprises applying a dense clustering, spectral clustering, k-means clustering, hierarchical clustering, and/or agglomerative clustering.

29. The method of claim 28, wherein the hierarchical clustering is performed using a Louvain community detection algorithm.

30. The method of claim 23, further comprising:
determining an GC TME type of a second subject, wherein the GC TME type of the second subject is identified using the updated GC TME signature clusters, wherein the identifying comprises:
determining an GC TME signature of the second subject from RNA expression data obtained by sequencing a biological sample obtained from the second subject;
associating the GC TME signature of the second subject with a particular one of the plurality of the updated GC TME signature clusters; and
identifying the GC TME type for the second subject as the GC TME type corresponding to the particular one of the plurality of updated GC TME signature clusters to which the GC TME signature of the second subject is associated.

31. The method of claim 1, wherein the plurality of a plurality of GC TME types comprises:
GC TME type A, GC TME type B, GC TME type C, GC TME type D, and GC TME type E.

32. The method of claim 1, further comprising:
identifying the subject as having tertiary lymphoid structures (TLS) when the subject is identified as having GC TME type E.

33. The method of claim 1, further comprising:
identifying the subject as having an increased likelihood of having a good prognosis, optionally, as measured by overall survival (OS) or progression-free survival (PFS) when the subject is identified as having GC TME type E.

34. The method of claim 1, further comprising:
administering an immunotherapy to the subject.

35. The method of claim 32, further comprising:
administering an immunotherapy to the subject when the subject is identified as having TLS.

36. A system, comprising:
at least one computer hardware processor; and
at least one computer-readable storage medium storing processor-executable instructions that, when executed by the at least one computer hardware processor, cause the at least one computer hardware processor to perform a method for identifying a gastric cancer (GC) tumor microenvironment (TME) type for a subject, the method comprising:
obtaining RNA expression data for the subject, the RNA expression data indicating RNA expression levels for at least some genes in each group of at least some of a plurality of gene groups listed in Table 1;
generating a GC TME signature for the subject using the RNA expression data, the GC TME signature comprising gene group scores for respective gene groups in the at least some of the plurality of gene groups, the generating comprising:
determining the gene group scores using the RNA expression levels; and
identifying, using the GC TME signature and from among a plurality of GC TME types, a GC TME type for the subject.

37. At least one computer-readable storage medium storing processor-executable instructions that, when executed by the at least one computer hardware processor, cause the at least one computer hardware processor to perform a method for identifying a gastric cancer (GC) tumor microenvironment (TME) type for a subject, the method comprising:
obtaining RNA expression data for the subject, the RNA expression data indicating RNA expression levels for at least some genes in each group of at least some of a plurality of gene groups listed in Table 1;
generating a GC TME signature for the subject using the RNA expression data, the GC TME signature comprising gene group scores for respective gene groups in the at least some of the plurality of gene groups, the generating comprising:
determining the gene group scores using the RNA expression levels; and
identifying, using the GC TME signature and from among a plurality of GC TME types, a GC TME type for the subject.
